(19) United States
(12) Patent Application Publication
(10) Pub. No.: US 2024/0415123 A1
(43) Pub. Date: Dec. 19, 2024

(54) METHODS AND COMPOSITIONS FOR ICAFOLIN HERBICIDE TOLERANCE

(71) Applicant: Monsanto Technology LLC, St. Louis, MO (US)

(72) Inventors: Anita Küpper, St. Louis, MO (US); Clayton T. Larue, Chesterfield, MO (US)

(21) Appl. No.: 18/735,465

(22) Filed: Jun. 6, 2024

Related U.S. Application Data
(60) Provisional application No. 63/508,094, filed on Jun. 14, 2023.
(60) Provisional application No. 63/606,492, filed on Dec. 5, 2023.

(51) Int. Cl. A01N 43/80; A01P 13/00; C12N 9/02; C12N 15/82

(52) U.S. Cl. CPC A01N 43/80; A01P 13/00; C12N 9/0071; C12N 15/8274

(57) ABSTRACT

The present disclosure relates to the field of biotechnology and provides novel recombinant DNA molecules for conferring tolerance to icafolin herbicides. The present disclosure also provides herbicide tolerant transgenic plants, seeds, cells, and plant parts comprising the recombinant DNA molecules, and methods of using the same.

### CROSS-REFERENCE TO RELATED APPLICATIONS

This application claims the priority of U.S. Provisional Application Ser. No. 63/508,094, filed Jun. 14, 2023, and U.S. Provisional Application Ser. No. 63/606,492, filed Dec. 5, 2023, the entire disclosures of each of which are incorporated herein by reference.

### INCORPORATION OF SEQUENCE LISTING

The sequence listing contained in the file named “MONS562US_ST26.xml”, which is 77 kilobytes (measured in MS-Windows®) and was created on Jun. 5, 2024, is filed herewith by electronic submission, and is incorporated herein by reference in its entirety.

### FIELD OF THE INVENTION

The present disclosure relates to the field of biotechnology. More specifically, the disclosure relates to recombinant DNA molecules encoding enzymes that provide tolerance to icafolin herbicides.

### BACKGROUND

Agricultural crop production often utilizes transgenic traits created using the methods of biotechnology. A heterologous gene, also known as a transgene, can be introduced into a plant to produce a transgenic trait. Expression of the transgene in the plant confers a trait, such as herbicide tolerance, to the plant. Non-limiting examples of transgenic herbicide tolerance traits include glyphosate tolerance, glufosinate tolerance, dicamba tolerance, and PPO herbicide tolerance. With the increase of weed species resistant to the commonly used herbicides, new herbicide tolerance traits are needed in the field. Herbicides of particular interest include icafolin herbicides. Icafolin herbicides provide control of a spectrum of herbicide-resistant weeds, thus making a trait conferring tolerance to these herbicides particularly useful in a cropping system. In particular embodiments, icafolin tolerance traits may be combined with one or more other herbicide-tolerance trait(s).

### SUMMARY

In one aspect, the present disclosure provides a recombinant DNA molecule comprising a heterologous promoter operably linked to a polynucleotide sequence that encodes a polypeptide comprising an amino acid sequence having at least about 94% sequence identity to SEQ ID NO:3, SEQ ID NO:4, SEQ ID NO:16, SEQ ID NO:17, SEQ ID NO:23, SEQ ID NO: 26, SEQ ID NO: 27, or SEQ ID NO:28 wherein the heterologous promoter is functional in a plant cell. In one embodiment, the encoded polypeptide confers tolerance to an icafolin herbicide. In another embodiment, the polynucleotide sequence comprises a nucleic acid sequence having at least about 95% sequence identity to SEQ ID NO:8, SEQ ID NO:9, SEQ ID NO:18, SEQ ID NO:19, SEQ ID NO:24, SEQ ID NO:29, SEQ ID NO:30, or SEQ ID NO:31, or at least about 85% sequence identity to SEQ ID NO:12, SEQ ID NO:13, SEQ ID NO:20, SEQ ID NO:21, SEQ ID NO:22, SEQ ID NO:25, SEQ ID NO:32, SEQ ID NO: 33, SEQ ID NO:34, SEQ ID NO: 35, or SEQ ID NO: 36. In yet another embodiment, the encoded polypeptide comprises the amino acid sequence of SEQ ID NO:3, SEQ ID NO:4, SEQ ID NO:16, SEQ ID NO: 17, SEQ ID NO:23, SEQ ID NO: 26, SEQ ID NO: 27, or SEQ ID NO:28. In still yet another embodiment, the nucleic acid sequence is selected from the group consisting of SEQ ID NO:8, SEQ ID NO:9, SEQ ID NO:12, SEQ ID NO:13, SEQ ID NO:18, SEQ ID NO:19, SEQ ID NO:20, SEQ ID NO:21, SEQ ID NO:22, SEQ ID NO:24, SEQ ID NO:25, SEQ ID NO: 29, SEQ ID NO:30, SEQ ID NO:31, SEQ ID NO:32, SEQ ID NO:33, SEQ ID NO:34, SEQ ID NO:35, and SEQ ID NO: 36. The polynucleotide sequence, in one embodiment, further comprises a nucleic acid sequence that encodes a targeting sequence that functions to localize the encoded polypeptide within a cell. The icafolin herbicide, in another embodiment, is selected from the group consisting of: methyl(2R*,4R*)-4-[[(5S)-3-(3,5-difluorophenyl)-5-vinyl-4H-isoxazole-5-carbonyl]amino]tetrahydrofuran-2-carboxylate; methyl(2R,4R)-4-[[(5S)-3-(3,5-difluorophenyl)-5-vinyl-4H-isoxazole-5-carbonyl]-amino]tetrahydrofuran-2-carboxylate; methyl (2S,4S)-4-[[(5S)-3-(3,5-difluorophenyl)-5-vinyl-4H-isoxazole-5-carbonyl]amino]tetrahydrofuran-2-carboxylate; (2R*,4R*)-4-[[(5S)-3-(3,5-difluorophenyl)-5-vinyl-4H-isoxazole-5-carbonyl]amino]tetrahydrofuran-2-carboxylic acid; (2R,4R)-4-[[(5S)-3-(3,5-difluorophenyl)-5-vinyl-4H-isoxazole-5-carbonyl]amino]tetrahydrofuran-2-carboxylic acid; (2S,4S)-4-[[(5S)-3-(3,5-difluorophenyl)-5-vinyl-4H-isoxazole-5-carbonyl]amino]tetrahydrofuran-2-carboxylic acid; and combinations of any thereof. In yet another embodiment, the present disclosure provides a DNA construct comprising a recombinant DNA molecule as described herein. In one embodiment, the DNA construct may comprise a recombinant DNA molecule comprising a heterologous promoter operably linked to a polynucleotide sequence that encodes a polypeptide comprising an amino acid sequence having at least about 94% sequence identity to SEQ ID NO:3, SEQ ID NO:4, SEQ ID NO:16, SEQ ID NO:17, SEQ ID NO:23, SEQ ID NO: 26, SEQ ID NO: 27, or SEQ ID NO:28 wherein the heterologous promoter is functional in a plant cell. In another embodiment, a recombinant DNA molecule of the present disclosure is integrated into the genome of a transgenic plant, seed, plant part, or cell.

In another aspect, the present disclosure provides a transgenic plant, seed, cell, or plant part comprising a recombinant DNA molecule comprising a heterologous promoter operably linked to a polynucleotide sequence that encodes a polypeptide comprising an amino acid sequence having at least about 85% sequence identity to SEQ ID NO:3, SEQ ID NO:4, SEQ ID NO:16, SEQ ID NO: 17, SEQ ID NO:23, SEQ ID NO: 26, SEQ ID NO: 27, or SEQ ID NO:28. In one embodiment, the transgenic plant, seed, cell, or plant part comprises tolerance to an icafolin herbicide. In another embodiment, the transgenic plant, seed, cell, or plant part is a soybean, a maize, a cotton, a barley, a sorghum, a rice, a wheat, a sugar cane, a sugar beet, an alfalfa, or a Brassica plant, seed, cell, or plant part. In yet another embodiment, the icafolin herbicide is selected from the group consisting of: methyl(2R*,4R*)-4-[[(5S)-3-(3,5-difluorophenyl)-5-vinyl-4H-isoxazole-5-carbonyl]amino]tetrahydrofuran-2-carboxylate; methyl(2R,4R)-4-[[(5S)-3-(3,5-difluorophenyl)-5-vinyl-4H-isoxazole-5-carbonyl]-amino] tetrahydrofuran-2-carboxylate; methyl (2S,4S)-4-[[(5S)-3-(3,5-difluorophenyl)-5-vinyl-4H-isoxazole-5-carbonyl]amino]tetrahydrofuran-2-carboxylate; (2R*,4R*)-4-[[(5S)-3-(3,5-difluorophenyl)-5-vinyl-4H-isoxazole-5-carbonyl]amino]tetrahydrofuran-2-carboxylic acid; (2R,4R)-4-[[(5S)-3-(3,5-difluorophenyl)-5-vinyl-4H-isoxazole-5-carbonyl]amino] tetrahydrofuran-2-carboxylic acid; (2S,4S)-4-[[(5S)-3-(3,5-difluorophenyl)-5-vinyl-4H-isoxazole-5-carbonyl]amino]tetrahydrofuran-2-carboxylic acid; and combinations of any thereof. In still yet another embodiment, a transgenic plant, seed, cell, or plant part of the present disclosure may comprise tolerance to at least one additional herbicide.

In yet another aspect, the present disclosure provides a method of conferring herbicide tolerance to a plant, seed, cell, or plant part, the method comprising expressing a recombinant DNA molecule of the present disclosure in the plant, seed, cell, or plant part. In one embodiment, the recombinant DNA molecule comprises a heterologous promoter operably linked to a polynucleotide sequence that encodes a polypeptide comprising an amino acid sequence having at least about 94% sequence identity to SEQ ID NO:3, SEQ ID NO:4, SEQ ID NO:16, SEQ ID NO:17, SEQ ID NO:23, SEQ ID NO: 26, SEQ ID NO: 27, or SEQ ID NO:28 wherein the heterologous promoter is functional in a plant cell. In another embodiment, the plant, seed, cell, or plant part comprises tolerance to an icafolin herbicide. In yet another embodiment, the icafolin herbicide is selected from the group consisting of: methyl(2R*,4R*)-4-[[(5S)-3-(3,5-difluorophenyl)-5-vinyl-4H-isoxazole-5-carbonyl]amino] tetrahydrofuran-2-carboxylate; methyl(2R,4R)-4-[[(5S)-3-(3,5-difluorophenyl)-5-vinyl-4H-isoxazole-5-carbonyl]-amino]tetrahydrofuran-2-carboxylate; methyl (2S,4S)-4-[1[(5S)-3-(3,5-difluorophenyl)-5-vinyl-4H-isoxazole-5-carbonyl]amino]tetrahydrofuran-2-carboxylate; (2R*,4R*)-4-[[(5S)-3-(3,5-difluorophenyl)-5-vinyl-4H-isoxazole-5-carbonyl]amino] tetrahydrofuran-2-carboxylic acid; (2R,4R)-4-[[(5S)-3-(3,5-difluorophenyl)-5-vinyl-4H-isoxazole-5-carbonyl]amino]tetrahydrofuran-2-carboxylic acid; (2S,4S)-4-[[(5S)-3-(3,5-difluorophenyl)-5-vinyl-4H-isoxazole-5-carbonyl]amino]tetrahydrofuran-2-carboxylic acid; and combinations of any thereof.

In still yet another aspect, the present disclosure provides a method of producing a transgenic plant or part thereof, the method comprising: a) introducing a recombinant DNA molecule comprising a heterologous promoter operably linked to a polynucleotide sequence that encodes a polypeptide comprising an amino acid sequence having at least about 85% sequence identity to SEQ ID NO:3, SEQ ID NO:4, SEQ ID NO:16, SEQ ID NO:17, SEQ ID NO:23, SEQ ID NO: 26, SEQ ID NO: 27, or SEQ ID NO:28 into a plant cell; and b) regenerating the transgenic plant or part thereof from the cell or a descendant cell thereof that comprises the recombinant DNA molecule. In one embodiment, the method may further comprise selecting a regenerated transgenic plant comprising tolerance to an icafolin herbicide. In another embodiment, the icafolin herbicide is selected from the group consisting of: methyl(2R*,4R*)-4-[[(5S)-3-(3,5-difluorophenyl)-5-vinyl-4H-isoxazole-5-carbonyl]amino] tetrahydrofuran-2-carboxylate; methyl(2R,4R)-4-[[(5S)-3-(3,5-difluorophenyl)-5-vinyl-4H-isoxazole-5-carbonyl]-amino]tetrahydrofuran-2-carboxylate; methyl (2S,4S)-4-[[(5S)-3-(3,5-difluorophenyl)-5-vinyl-4H-isoxazole-5-carbonyl]amino]tetrahydrofuran-2-carboxylate; (2R*,4R*)-4-[[(5S)-3-(3,5-difluorophenyl)-5-vinyl-4H-isoxazole-5-carbonyl]amino] tetrahydrofuran-2-carboxylic acid; (2R,4R)-4-[[(5S)-3-(3,5-difluorophenyl)-5-vinyl-4H-isoxazole-5-carbonyl]amino]tetrahydrofuran-2-carboxylic acid; (2S,4S)-4-[[(5S)-3-(3,5-difluorophenyl)-5-vinyl-4H-isoxazole-5-carbonyl]amino]tetrahydrofuran-2-carboxylic acid; and combinations of any thereof. In yet another embodiment, the method may further comprise crossing the regenerated transgenic plant with itself or with a second plant to produce seed. In still yet another embodiment, the encoded polypeptide comprises the amino acid sequence of SEQ ID NO:3, SEQ ID NO:4, SEQ ID NO:16, SEQ ID NO:17, SEQ ID NO:23, SEQ ID NO:26, SEQ ID NO:27, or SEQ ID NO:28. In one embodiment, the polynucleotide sequence comprises a nucleic acid sequence having at least about 95% sequence identity to SEQ ID NO:8, SEQ ID NO:9, SEQ ID NO:18, SEQ ID NO:19, SEQ ID NO:24, SEQ ID NO:29, SEQ ID NO:30, or SEQ ID NO:31 or at least about 85% sequence identity to SEQ ID NO:12, SEQ ID NO:13, SEQ ID NO:20; SEQ ID NO:21, SEQ ID NO:22, or SEQ ID NO:25, SEQ ID NO: 32, SEQ ID NO: 33, SEQ ID NO:34, SEQ ID NO: 35, or SEQ ID NO:36. In another embodiment, the method may further comprise crossing the regenerated transgenic plant, or a descendant plant thereof that comprises the recombinant DNA molecule, with itself or a second plant to produce seed; wherein the seed comprises the recombinant DNA molecule. In yet another embodiment, the present disclosure provides a seed produced by the methods described herein. The present disclosure further provides, in still yet another embodiment, a transgenic plant or part thereof produced by the methods described herein, wherein the transgenic plant or part thereof comprises the recombinant DNA molecule.

In one aspect, the present disclosure provides a method for controlling weeds in a plant growth area that comprises a transgenic plant or seed of the present disclosure, the method comprising contacting the plant growth area with an icafolin herbicide, wherein the transgenic plant or seed is tolerant to the icafolin herbicide, and wherein weeds are controlled in the plant growth area. In one embodiment, the icafolin herbicide is selected from the group consisting of: methyl(2R*,4R*)-4-[[(5S)-3-(3,5-difluorophenyl)-5-vinyl-4H-isoxazole-5-carbonyl]amino]tetrahydrofuran-2-carboxylate; methyl(2R,4R)-4-[[(5S)-3-(3,5-difluorophenyl)-5-vinyl-4H-isoxazole-5-carbonyl]-amino]tetrahydrofuran-2-carboxylate; methyl (2S,4S)-4-[[(5S)-3-(3,5-difluorophenyl)-5-vinyl-4H-isoxazole-5-carbonyl]amino]tetrahydrofuran-2-carboxylate; (2R*,4R*)-4-[[(5S)-3-(3,5-difluorophenyl)-5-vinyl-4H-isoxazole-5-carbonyl]amino]tetrahydrofuran-2-carboxylic acid; (2R,4R)-4-[[(5S)-3-(3,5-difluorophenyl)-5-vinyl-4H-isoxazole-5-carbonyl]amino]tetrahydrofuran-2-carboxylic acid; (2S,4S)-4-[[(5S)-3-(3,5-difluorophenyl)-5-vinyl-4H-isoxazole-5-carbonyl]amino]tetrahydrofuran-2-carboxylic acid; and combinations of any thereof. In another embodiment, the transgenic plant or seed is a soybean, a maize, a cotton, a barley, a sorghum, a rice, a wheat, a sugar cane, a sugar beet, an alfalfa, or a Brassica plant or seed.

In another aspect, the present disclosure provides a method of identifying a plant having tolerance to an icafolin herbicide and to at least one additional herbicide, the method comprising: a) obtaining a transgenic plant of the present disclosure comprising tolerance to an icafolin herbicide and to at least one additional herbicide; b) applying the at least one additional herbicide to the plant or a part thereof; and c) identifying the plant as exhibiting tolerance to the at least one additional herbicide.

In yet another aspect, the present disclosure provides a method for reducing the development of herbicide tolerant weeds in a plant growth area comprising a transgenic plant or seed of the present disclosure comprising tolerance to an icafolin herbicide and to at least one additional herbicide, the method comprising contacting the plant growth area with the icafolin herbicide and at least one additional herbicide, wherein the transgenic plant or seed is tolerant to the icafolin herbicide and the at least one additional herbicide. In one embodiment, the icafolin herbicide is selected from the group consisting of: methyl(2R*,4R*)-4-[[(5S)-3-(3,5-difluorophenyl)-5-vinyl-4H-isoxazole-5-carbonyl]amino]tetrahydrofuran-2-carboxylate; methyl(2R,4R)-4-[[(5S)-3-(3,5-difluorophenyl)-5-vinyl-4H-isoxazole-5-carbonyl]-amino]tetrahydrofuran-2-carboxylate; methyl (2S,4S)-4-[[(5S)-3-(3,5-difluorophenyl)-5-vinyl-4H-isoxazole-5-carbonyl]amino] tetrahydrofuran-2-carboxylate; (2R*,4R*)-4-[[(5S)-3-(3,5-difluorophenyl)-5-vinyl-4H-isoxazole-5-carbonyl]amino]tetrahydrofuran-2-carboxylic acid; (2R,4R)-4-[[(5S)-3-(3,5-difluorophenyl)-5-vinyl-4H-isoxazole-5-carbonyl]amino]tetrahydrofuran-2-carboxylic acid; (2S,4S)-4-[[(5S)-3-(3,5-difluorophenyl)-5-vinyl-4H-isoxazole-5-carbonyl]amino]tetrahydrofuran-2-carboxylic acid; and combinations of any thereof. In another embodiment, the at least one additional herbicide is selected from the group consisting of: an ACCase inhibitor, an ALS inhibitor, an EPSPS inhibitor, a synthetic auxin, a photosynthesis inhibitor, a glutamine synthesis inhibitor, a HPPD inhibitor, a PPO inhibitor, a PDS inhibitor, and a long-chain fatty acid inhibitor.

Any aspect or embodiment of the present disclosure may be used in combination with any other aspect or embodiment described herein.

### BRIEF DESCRIPTION OF THE SEQUENCES

SEQ ID NO:1 is an amino acid sequence of an Amaranthus palmeri CYP12 protein.
SEQ ID NO:2 is an amino acid sequence of an Amaranthus palmeri CYP13 protein.
SEQ ID NO:3 is an amino acid sequence of an Amaranthus palmeri CYP14 protein.
SEQ ID NO:4 is an amino acid sequence of an Amaranthus palmeri CYP15 protein.
SEQ ID NO:5 is an amino acid sequence of a variant of CYP12 protein comprising a serine amino acid inserted at position 2.
SEQ ID NO:6 is an Amaranthus palmeri nucleotide sequence encoding SEQ ID NO:1.
SEQ ID NO:7 is an Amaranthus palmeri nucleotide sequence encoding SEQ ID NO:2
SEQ ID NO:8 is an Amaranthus palmeri nucleotide sequence encoding SEQ ID NO:3.
SEQ ID NO:9 is an Amaranthus palmeri nucleotide sequence encoding SEQ ID NO:4
SEQ ID NO:10 is a nucleotide sequence encoding SEQ ID NO:5, codon optimized for dicot expression.
SEQ ID NO:11 is a nucleotide sequence encoding SEQ ID NO:2, codon optimized for dicot expression.
SEQ ID NO:12 is a nucleotide sequence encoding SEQ ID NO:3, codon optimized for dicot expression.
SEQ ID NO:13 is a nucleotide sequence encoding SEQ ID NO:4, codon optimized for dicot expression.
SEQ ID NO:14 is a nucleotide sequence for the expression element comprising the promoter, leader, and intron of Arabidopsis thaliana Ubiquitin 3.
SEQ ID NO:15 is a nucleotide sequence for the terminator sequence of Medicago truncatula photosystem II.
SEQ ID NO:16 is an amino acid sequence of an Amaranthus palmeri CYP21 protein.
SEQ ID NO:17 is an amino acid sequence of an Amaranthus palmeri CYP52 protein.
SEQ ID NO:18 is an Amaranthus palmeri nucleotide sequence encoding SEQ ID NO:16.
SEQ ID NO:19 is an Amaranthus palmeri nucleotide sequence encoding SEQ ID NO:17.
SEQ ID NO:20 is a nucleotide sequence encoding SEQ ID NO:16, codon optimized for dicot expression.
SEQ ID NO:21 is a nucleotide sequence encoding SEQ ID NO:17, codon optimized for dicot expression.
SEQ ID NO:22 is a nucleotide sequence encoding SEQ ID NO:17, codon optimized for monocot expression.
SEQ ID NO:23 is an amino acid sequence of an Amaranthus tuberculatus CYP28 protein.
SEQ ID NO:24 is an Amaranthus tuberculatus nucleotide sequence encoding SEQ ID NO:23.
SEQ ID NO:25 is a nucleotide sequence encoding SEQ ID NO:23, codon optimized for dicot expression.
SEQ ID NO:26 is an amino acid sequence of an Amaranthus cruentus CYP34 protein.
SEQ ID NO:27 is an amino acid sequence of an Amaranthus palmeri CYP55 protein.
SEQ ID NO:28 is an amino acid sequence of an Amaranthus palmeri CYP 56 protein.
SEQ ID NO:29 is an Amaranthus cruentus nucleotide sequence encoding SEQ ID NO:26.
SEQ ID NO:30 is an Amaranthus palmeri nucleotide sequence encoding SEQ ID NO:27.
SEQ ID NO:31 is an Amaranthus palmeri nucleotide sequence encoding SEQ ID NO:28.
SEQ ID NO:32 is a nucleotide sequence encoding SEQ ID NO:26, codon optimized for dicot expression.
SEQ ID NO:33 is a nucleotide sequence encoding SEQ ID NO:27, codon optimized for dicot expression.
SEQ ID NO:34 is a nucleotide sequence encoding SEQ ID NO:27, codon optimized for monocot expression.
SEQ ID NO:35 is a nucleotide sequence encoding SEQ ID NO:28, codon optimized for dicot expression.
SEQ ID NO:36 is a nucleotide sequence encoding SEQ ID NO:28, codon optimized for monocot expression.

### BRIEF DESCRIPTION OF THE DRAWINGS

FIG. 1: Shows representative soybean plants following various icafolin treatment regimens. Photographs were taken 3 weeks post treatment.

### DETAILED DESCRIPTION

The following is a detailed description provided to aid those skilled in the art in practicing the embodiments of the present disclosure. Modifications and variations to the embodiments described herein can be made without departing from the spirit or scope of the present disclosure.

The present disclosure provides novel, recombinant DNA molecules that encode cytochrome P450 enzymes (CYPs) that confer tolerance to icafolin herbicides. CYP enzymes are members of a super family of enzymes reported to catalyze a large number of reactions on different substrates and are found in both prokaryotic and eukaryotic species. The cytochrome P450 superfamily is the largest enzymatic protein family in plants with hundreds of members that are involved in a number of metabolic pathways with distinct and complex functions (Xu et. al., The cytochrome P450 superfamily: Key players in plant development and defense, J. of Integrative Agric., 14:9, 1673-1686, 2015). Some CYP enzymes have been implicated in herbicide resistance observed in certain weed species (see, e.g., Siminszky, B. Plant cytochrome P450-mediated herbicide metabolism. Phytochem Rev 5, 445-458, 2006). The plant P450 enzymes are named and classified into the CYP71-CYP99 and the CYP701-CYP999 gene families (see Nelson DR. Cytochrome P450 diversity in the tree of life. Biochim Biophys Acta Proteins Proteom. 1866 (1):141-154. 2018).

CYP14 and CYP15 belong to the CYP81E subfamily of cytochrome P450 enzymes. CYP81E8 genes have been identified as being differentially expressed in a population of waterhemp (Amaranthus tuberculatus) that was resistant to the herbicide 2,4-dichlorophenoxyacetic acid (2,4-D) (see PCT Publication No. WO2022/051340 and Giacomini, et al., Coexpression Clusters and Allele-Specific Expression in Metabolism-Based Herbicide Resistance. Genome Biol Evol.; 12:2267-2278, 2020). Similarly, increased expression of a CYP81E8 gene was also noted in an Amaranthus palmeri population that was resistant to the 4-hydroxyphenylpyruvate dioxygenase (HPPD)-inhibitor herbicide tembotrione (see Kuepper, A., Molecular genetics of herbicide resistance in palmer amaranth (Amaranthus palmeri): Metabolic tembotrione resistance and geographic origin of glyphosate resistance PhD Thesis., Colorado State University, Fort Collins, CO, 2018 and Aarthy et. al., Front. Agron., Vol 4, Rapid metabolism, and increased expression of CYP81E8 gene confer high level of resistance to tembotrione in a multiple-resistant Palmer amaranth, 2022 https://doi.org/10.3389/fagro.2022.1010292).

In certain embodiments, the present disclosure further provides vectors and expression cassettes encoding CYP proteins for expression in cells and plants. Methods for producing cells and plants tolerant to icafolin herbicides are also provided. The disclosure further provides methods and compositions for the use of protein engineering techniques and bioinformatic tools to obtain and improve enzymes that confer icafolin tolerance.

### A. Icafolin Herbicides

Icafolin is a novel post-emergence isoxazole herbicide that acts on dicot (broadleaf) and monocot (narrow leaf/grass) weeds. Icafolin has the following structure:

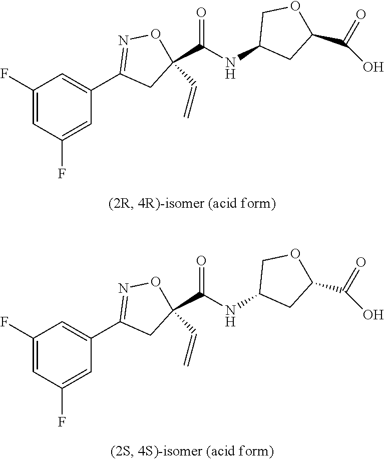 [SMILES: C=C[C@]1(C(=O)N[C@H]2CO[C@@H](C(=O)O)C2)CC(c2cc(F)cc(F)c2)=NO1 ; C=C[C@]1(C(=O)N[C@@H]2CO[C@H](C(=O)O)C2)CC(c2cc(F)cc(F)c2)=NO1]

In certain embodiments, icafolin can be applied as a mixture of two isomers (2R, 4R or 2S, 4S), or can be applied as individual isomers. In some embodiments, icafolin can be applied in the acid form (shown above) or as a methyl ester (shown below).

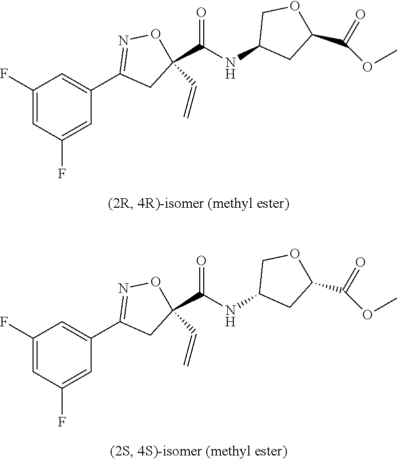 [SMILES: C=C[C@]1(C(=O)N[C@H]2CO[C@@H](C(=O)OC)C2)CC(c2cc(F)cc(F)c2)=NO1 ; C=C[C@]1(C(=O)N[C@@H]2CO[C@H](C(=O)OC)C2)CC(c2cc(F)cc(F)c2)=NO1]

Non-limiting examples of an icafolin herbicide that may be used according to the embodiments of the present disclosure include methyl(2R*,4R*)-4-[[(5S)-3-(3,5-difluorophenyl)-5-vinyl-4H-isoxazole-5-carbonyl]amino]tetrahydrofuran-2-carboxylate; methyl(2R,4R)-4-[[(5S)-3-(3,5-difluorophenyl)-5-vinyl-4H-isoxazole-5-carbonyl]-amino]tetrahydrofuran-2-carboxylate; methyl(2S,4S)-4-[[(5S)-3-(3,5-difluorophenyl)-5-vinyl-4H-isoxazole-5-carbonyl]amino]tetrahydrofuran-2-carboxylate; (2R*,4R*)-4-[[(5S)-3-(3,5-difluorophenyl)-5-vinyl-4H-isoxazole-5-carbonyl]amino]tetrahydrofuran-2-carboxylic acid; (2R,4R)-4-[[(5S)-3-(3,5-difluorophenyl)-5-vinyl-4H-isoxazole-5-carbonyl]amino]tetrahydrofuran-2-carboxylic acid; (2S,4S)-4-[[(5S)-3-(3,5-difluorophenyl)-5-vinyl-4H-isoxazole-5-carbonyl]amino]tetrahydrofuran-2-carboxylic acid, and combinations of any thereof.

Icafolin and related compounds are described in U.S. Patent Application Publication No. 2021/292312, the disclosure of which is incorporated herein by reference in its entirety.

### B. Recombinant Polynucleotide Molecules and Encoded Proteins

In specific aspects, the present disclosure provides recombinant DNA molecules and proteins. As used herein, the term “recombinant” refers to a non-naturally occurring DNA, protein, cell, seed, or organism that is the result of genetic engineering and as such would not normally be found in nature. As used herein, a “recombinant DNA molecule” is a DNA molecule comprising a DNA sequence that does not naturally occur in nature and as such is the result of human intervention, such as a DNA molecule comprised of at least two DNA molecules heterologous to each other. In one embodiment, a recombinant DNA molecule is a DNA molecule provided herein encoding a protein conferring tolerance to an icafolin herbicide operably linked to a heterologous regulatory or other element, such as a heterologous promoter. As used herein, a “recombinant protein” is a protein comprising an amino acid sequence that does not naturally occur and as such is the result of human intervention. In one embodiment, a recombinant protein may be an engineered protein or a chimeric protein. In another embodiment, a recombinant cell, seed, or organism may be a cell, seed, or organism comprising transgenic DNA. A recombinant cell, seed, or organisms may be, for example, a transgenic cell, seed, plant, or plant part produced as a result of plant transformation and comprising a recombinant DNA molecule.

As used herein the term “heterologous” refers to a polynucleotide molecule or protein that is not naturally present, or is not naturally present in the same form or structure, in the cell being genetically modified, without human intervention. For example, a heterologous polynucleotide molecule may not naturally occur in the plant species being transformed or modified, or may be expressed in a manner or genomic context that differs from the natural expression pattern or genomic context found in the species being transformed or modified. For example, in some embodiments the heterologous polynucleotide molecule may be overexpressed. In particular embodiments, the heterologous polynucleotide molecule may be the combination of two or more polynucleotide molecules, wherein such a combination is not normally found in nature. The two polynucleotide molecules may, in certain embodiments, be derived from different species or may be derived from different genes, such as, different genes from the same species or the same genes from different species. In some embodiments, a heterologous polynucleotide molecule may comprise two polynucleotide sequences that are not found juxtaposed or operably linked in any naturally occurring polynucleotide molecule. The heterologous polynucleotide molecule, in further embodiments, may comprise a promoter or other regulatory sequence operably linked to a transcribable polynucleotide sequence, wherein the promoter or other regulatory sequence and the transcribable polynucleotide sequence are not operably linked in any naturally occurring polynucleotide molecule.

As used herein, the phrase “not normally found in nature” means not found in nature without human intervention. A recombinant polynucleotide or protein molecule may comprise, in certain embodiments, polynucleotide or protein sequences that are (i) separated from other polynucleotide or protein sequence(s) that exist in proximity to each other in nature, or (ii) adjacent to (or contiguous with) other polynucleotide or protein sequences that are not naturally in proximity to each other. Recombinant polynucleotide molecules or proteins may also refer, in some embodiments, to a polynucleotide or protein molecule or sequence that has been genetically engineered or constructed outside of a cell. For example, a recombinant polynucleotide molecule may comprise any engineered or man-made plasmid, vector, or construct, and may include a linear or circular polynucleotide molecule. Such plasmids, vectors, or constructs, may contain, in certain embodiments, various maintenance elements including, for example, a prokaryotic origin of replication or selectable marker gene, as well as one or more transgenes or expression cassettes.

As used herein, the term “isolated” refers to at least partially separating a molecule from other molecules typically associated with it in its natural state. In one embodiment, the term isolated refers to a DNA molecule that is separated from the nucleic acids that normally flank the DNA molecule in its natural state. For example, a DNA molecule encoding a protein that is naturally present in a particular plant species would be an isolated DNA molecule if it was not within the DNA of the plant species from which the DNA molecule encoding the protein is naturally found. Thus, a DNA molecule fused to or operably linked to one or more other DNA molecule(s) with which it would not be associated in nature, for example as the result of recombinant DNA or plant transformation techniques, is considered isolated herein. Such molecules are considered isolated even when integrated into the chromosome of a host cell or present in a nucleic acid solution with other DNA molecules.

As used herein, the term “protein-coding DNA molecule” refers to a DNA molecule comprising a nucleotide sequence that encodes a protein. As used herein, a “protein-coding sequence” refers to a DNA sequence that encodes a protein. As used herein a “sequence” refers to a sequential arrangement of nucleotides or amino acids. In certain embodiments, the boundaries of a protein-coding sequence may be determined by a translation start codon at the 5′-terminus and a translation stop codon at the 3′-terminus. In some embodiments, a protein-coding molecule may comprise a DNA sequence encoding a protein sequence. As used herein, “transgene expression”, “expressing a transgene”, “protein expression”, and “expressing a protein” refer to the production of a protein through the process of transcribing a DNA molecule into messenger RNA (mRNA) and translating the mRNA into polypeptide chains, which are ultimately folded into proteins. A protein-coding DNA molecule may be, in certain embodiments, operably linked to a heterologous promoter in a DNA construct for expressing the protein in a cell transformed with the recombinant DNA molecule. As used herein, “operably linked” refers to two DNA molecules linked in manner so that one may affect the function of the other. Operably-linked DNA molecules may be part of a single contiguous molecule and may or may not be adjacent. For example, a promoter is operably linked with a protein-coding DNA molecule in a DNA construct where the two DNA molecules are so arranged that the promoter may affect the expression of the transgene.

As used herein, a “DNA construct” refers to a recombinant DNA molecule comprising two or more heterologous DNA sequences. DNA constructs are useful for transgene expression and may be comprised within vectors or plasmids. DNA constructs may be used in vectors for the purpose of transformation, that is the introduction of heterologous DNA into a host cell, to produce transgenic plants and cells, and as such may also be contained in the plastid DNA or genomic DNA of a transgenic plant, seed, cell, or plant part. As used herein, a “vector” refers to any recombinant DNA molecule that may be used for the purpose of bacterial or plant transformation. Recombinant DNA molecules provided by the present disclosure may, for example, be inserted into a vector as part of a construct having the recombinant DNA molecule operably linked to a gene expression element that functions in a plant to affect expression of the engineered protein encoded by the recombinant DNA molecule. General methods useful for manipulating DNA molecules for making and using recombinant DNA constructs and plant transformation vectors are well known in the art and described in detail in, for example, handbooks and laboratory manuals including MR Green and J Sambrook, “Molecular Cloning: A Laboratory Manual” (Fourth Edition) ISBN:978-1-936113-42-2, Cold Spring Harbor Laboratory Press, NY (2012). The components for a DNA construct or a vector comprising a DNA construct, in certain embodiments, may include one or more gene expression elements operably linked to a transcribable DNA sequence, such as the following: a promoter for the expression of an operably linked DNA, an operably linked protein-coding DNA molecule, and an operably linked 3′ untranslated region (UTR). Gene expression elements useful in practicing the embodiments of the present disclosure include, but are not limited to, one or more of the following types of elements: promoter, 5′ UTR, enhancer, leader, cis-acting element, intron, targeting sequence, 3′ UTR, and one or more selectable marker transgenes.

The DNA constructs of the present disclosure may include a promoter operably linked to a protein-coding DNA molecule provided by the present disclosure, whereby the promoter drives expression of the recombinant protein molecule. Promoters useful in practicing the embodiments of the present disclosure include those that function in a cell for expression of an operably linked polynucleotide, such as a bacterial promoter, a viral promoter, or a plant promoter. Plant promoters are varied and well known in the art and include, for instance, those that are inducible, viral, synthetic, constitutive, temporally regulated, spatially regulated, and/or spatio-temporally regulated.

In one embodiment of the present disclosure, a DNA construct provided herein includes a targeting sequence that is operably linked to a heterologous nucleic acid molecule encoding a polypeptide molecule that confers tolerance to an icafolin herbicide, whereby the targeting sequence facilitates localizing the polypeptide molecule within the cell. Targeting sequences are known in the art as signal sequences, targeting peptides, localization sequences, and transit peptides. Non-limiting examples of a targeting sequences include a chloroplast transit peptide (CTP), a mitochondrial targeting sequence (MTS), and a dual chloroplast and mitochondrial targeting peptide. By facilitating protein localization within the cell, the targeting sequence may increase the accumulation of a recombinant protein, protect the protein from proteolytic degradation, and/or enhance the level of herbicide tolerance, thereby reducing levels of injury in the transgenic cell, seed, or organism following herbicide application.

CTPs and other targeting molecules that may be used in connection with the present disclosure are known in the art and include, but are not limited to, the Arabidopsis thaliana EPSPS CTP (Klee et al., Mol Gen Genet. 210:437-442, 1987), the Petunia hybrida EPSPS CTP (della-Cioppa et al., PNAS 83:6873-6877, 1986), the maize cab-m7 signal sequence (Becker et al., Plant Mol Biol. 20:49-60, 1992; PCT WO 97/41228), a mitochondrial pre-sequence (e.g., Silva Filho et al., Plant Mol Biol 30:769-780, 1996), and the pea glutathione reductase signal sequence (Creissen et al., Plant J. 8:167-175, 1995; PCT WO 97/41228).

Recombinant DNA molecules of the present disclosure may be synthesized and modified by methods known in the art, either completely or in part, where it is desirable to provide sequences useful for DNA manipulation (such as restriction enzyme recognition sites or recombination-based cloning sites), plant-preferred sequences (such as plant-codon usage or Kozak consensus sequences), or sequences useful for DNA construct design (such as spacer or linker sequences). In particular embodiments, a recombinant DNA molecule or protein of the present disclosure may comprise or encode a sequence having at least about 70%, about 75%, about 80%, about 85%, about 90%, about 91%, about 92%, about 93%, about 94%, about 95%, about 96%, about 97%, about 98%, about 99%, or 100% sequence identity to any one of SEQ ID NOs:1-36, or fragments thereof, including any range derivable therebetween. In some embodiments, the recombinant DNA molecules or proteins of the present disclosure may confer icafolin herbicide tolerance when expressed in a plant cell. In certain embodiments, a sequence derived from any of SEQ ID NOs: 1-36 may have the activity of the base sequence from which it was derived, for example herbicide tolerance activity. As used herein, the term “percent sequence identity” or “% sequence identity” refers to the percentage of identical nucleotides or amino acids in a linear polynucleotide or polypeptide sequence of a reference (“query”) sequence (or its complementary strand) as compared to a test (“subject”) sequence (or its complementary strand) when the two sequences are optimally aligned (with appropriate nucleotide or amino acid insertions, deletions, or gaps totaling less than 20 percent of the reference sequence over the window of comparison). Optimal alignment of sequences for aligning a comparison window are well known to those skilled in the art and may be conducted by tools such as the local homology algorithm of Smith and Waterman, the homology alignment algorithm of Needleman and Wunsch, the search for similarity method of Pearson and Lipman, and by computerized implementations of these algorithms such as GAP, BESTFIT, FASTA, and TFASTA available as part of the Sequence Analysis software package of the GCG® Wisconsin Package® (Accelrys Inc., San Diego, CA), MEGAlign (DNAStar Inc., 1228 S. Park St., Madison, WI 53715), and MUSCLE (version 3.6) (Edgar, “MUSCLE: multiple sequence alignment with high accuracy and high throughput” Nucleic Acids Research 32(5):1792-7 (2004)) for instance with default parameters. An “identity fraction” for aligned segments of a test sequence and a reference sequence is the number of identical components that are shared by the two aligned sequences divided by the total number of components in the portion of the reference sequence segment being aligned, that is, the entire reference sequence or a smaller defined part of the reference sequence. Percent sequence identity is represented as the identity fraction multiplied by 100. The comparison of one or more sequences may be to a full-length sequence or a portion thereof, or to a longer sequence.

Recombinant DNA molecules or proteins of the present disclosure may comprise or encode a fragment of any of SEQ ID NOs:1-36. For example, the present disclosure provides DNA molecules comprising at least 50, at least 100, at least 150, at least 200, at least 250, at least 300, at least 350, at least 400, at least 450, at least 500, at least 550, at least 600, at least 650, at least 700, at least 750, at least 800, at least 850, at least 900, at least 950, at least 1000, at least 1100, at least 1200, at least 1300, at least 1400, at least 1500, at least 1600, at least 1700, at least 1800, at least 1900, at least 2000 or more contiguous nucleotides of SEQ ID NO:6-15, 18-22, 24-25, or 29-36. Such fragments of SEQ ID NO:6-15, 18-22, 24-25 or 29-36 may have the activity of the base sequence from which they are derived, for example herbicide tolerance activity. In some embodiments, proteins of the present disclosure provide fragments comprising at least 25, at least 50, at least 75, at least 100, at least 125, at least 150, at least 175, at least 200, at least 225, at least 250, at least 300, at least 325, at least 350, at least 375, at least 400, at least 425, at least 450, at least 475, at least 500, at least 525, at least 550, at least 575, at least 600, at least 625, at least 650, at least 675, at least 700, at least 725, at least 750, at least 775, at least 800, at least 825, at least 850, at least 875, at least 900, at least 925, at least 950, at least 975, at least 1000, or more contiguous amino acids of any of SEQ ID NO:1-5, 16, 17, 23 or 26-28. Such fragments of SEQ ID NO:1-5, 16, 17, 23 or 26-28 may have the activity of the base sequence from which they are derived, for example herbicide tolerance activity.

Engineered proteins may be produced by changing (that is, modifying) a wild-type protein to produce a new protein with modified characteristic(s) e.g. a particular cellular localization pattern, such as targeted to the chloroplast or mitochondria, or a novel combination of useful protein characteristics, such as altered Vₘₐₓ, Kₘ, Kᵢ, IC₅₀, k_{cat}, substrate specificity, inhibitor/herbicide specificity, substrate selectivity, the ability to interact with other components in the cell such as partner proteins or membranes, and protein stability, among others. In certain embodiments, modifications may be made at specific amino acid positions in a protein. In one embodiment, a substitution of the amino acid found at a particular amino acid position in nature (that is, in the wild-type protein) with a different amino acid may be made. Engineered proteins provided by the present disclosure thus provide a new protein with one or more altered protein characteristics relative to a similar protein found in nature. In one embodiment of the present disclosure, an engineered protein has altered protein characteristics, such as those that result in decreased sensitivity to one or more herbicides as compared to a similar wild-type protein, or an improved ability to confer herbicide tolerance to a transgenic plant expressing the engineered protein. In another embodiment, the present disclosure provides an engineered protein, and the recombinant DNA molecule encoding it, comprising at least one amino acid substitution selected from Table 1 and having at least about 70%, about 75%, about 80%, about 85%, about 90%, about 91%, about 92%, about 93%, about 94%, about 95%, about 96%, about 97%, about 98%, or about 99% sequence identity to any of the engineered protein sequences provided herein, including but not limited to those protein sequences comprising or encoded by any one of SEQ ID NOs:1-36, including all ranges derivable therebetween. Amino acid substitutions may be made as a single amino acid substitution in the protein or in combination with one or more other mutation(s), such as one or more other amino acid substitution(s), deletions, or additions. Mutations may be made by any method known to those of skill in the art.

TABLE 1
Amino Acid Substitutions.

| | Conservative | | Conservative |
|---|---|---|---|
| Residue | Substitutions | Residue | Substitutions |
| Ala | Ser | Leu | Ile; Val |
| Arg | Lys | Lys | Arg; Gln |
| Asn | Gln; His | Met | Leu; Ile |
| Asp | Glu | Phe | Met; Leu; Tyr |
| Gln | Asn | Ser | Thr; Gly |
| Cys | Ser | Thr | Ser; Val |
| Glu | Asp | Trp | Tyr |
| Gly | Pro | Tyr | Trp; Phe |
| His | Asn; Gln | Val | Ile; Leu |
| Ile | Leu; Val | | |

As used herein, “wild-type” means a naturally occurring similar, but not identical, version of a protein. A “wild-type DNA molecule” or “wild-type protein” is a naturally occurring version of a DNA molecule or protein, that is, a version of a DNA molecule or protein pre-existing in nature.

### C. Genetically Modified Plants Produced by Genetic Engineering

Various genetic engineering technologies have been developed and may be used by those of skill in the art to introduce transgenic or edited traits into plants. The methods generally involve the delivery of a polynucleotide sequence into a plant cell, which may typically be a heterologous or recombinant polynucleotide molecule. A heterologous or recombinant polynucleotide molecule may comprise, for example, at least one transgene, expression cassette, or RNA molecule, such as a guide RNA (gRNA). In specific embodiments, the heterologous or recombinant polynucleotide molecule may be part of a ribonucleoprotein (RNP) or a fragment thereof or a guide RNA. Expression of the heterologous or recombinant polynucleotide molecule may, for example, produce a gRNA/site-specific nuclease complex for genome editing. In certain embodiments, traits are introduced into plants by altering or introducing a single genetic locus or transgene into the genome of a plant. Methods of genetic engineering to modify, delete, or insert transgenes, edits, mutations, and polynucleotide sequences into the genomic DNA of plants are known in the art. Molecular methods of editing a plant cell genome or endogenous plant gene using a genome editing technique are known in the art. According to present embodiments, a polynucleotide or DNA molecule comprising or encoding genome editing tools or machinery, such as a guide RNA, site-specific nuclease, and/or template DNA molecule, may be introduced into a plant cell using the methods described herein.

As used herein, a “genetically modified” plant, plant part, plant tissue, or plant cell comprises a genetic modification. As used herein, a “genetic modification” refers to one or more transgenes, mutations, or edits introduced into the genome of a plant, plant part or plant cell using a transformation, mutagenesis, or genome editing technique. Apart from a genome editing technique, a mutagenesis technique may include any chemical, physical, radiological, or biological (e.g., transposon-mediated) mutagenesis technique or mutagen.

As used herein, a “transgenic” plant, plant part, plant tissue, or plant cell has an exogenous nucleic acid sequence, genome edit, or transgene integrated into the genome of the plant, plant part, plant tissue, or plant cell. As used herein, a “transgenic trait” refers to a characteristic or phenotype conveyed or conferred by the presence of a transgene, nucleic acid sequence, genome edit, or transgene incorporated into the plant genome. Because of such a genomic alteration, the transgenic plant is something distinctly different from the related wild-type plant and the transgenic trait is a trait not naturally found in the wild-type plant. In certain embodiments, transgenic plants of the present disclosure comprise the recombinant DNA molecules and engineered proteins provided by the herein.

Genome editing can be used to make one or more edits or mutations at a desired target site in the genome of a plant, such as to change expression or activity of one or more genes, or to integrate an insertion sequence or transgene at a desired location in a plant genome. Any site or locus within the genome of a plant may potentially be chosen for making a gene edit or site-directed integration of a transgene, construct, or transcribable DNA sequence. As used herein, a “target site” for genome editing or site-directed integration refers to the location of a polynucleotide sequence within a plant genome that is bound and cleaved by a site-specific nuclease to introduce a double-stranded break (DSB) or single-stranded nick into the nucleic acid backbone of the polynucleotide sequence and/or its complementary DNA strand within the plant genome. A target site may comprise, for example, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 29, or at least 30 consecutive nucleotides. A “target site” for an RNA-guided nuclease may comprise the sequence of either complementary strand of a double-stranded nucleic acid (DNA) molecule or chromosome at the target site. A site-specific nuclease may bind to a target site, such as via a non-coding guide RNA (e.g., without being limiting, a CRISPR RNA (crRNA) or a single-guide RNA (sgRNA) as described further herein). A non-coding guide RNA provided herein may be complementary to a target site (e.g., complementary to either strand of a double-stranded nucleic acid molecule or chromosome at the target site). It will be appreciated that perfect identity or complementarity may not be required for a non-coding guide RNA to bind or hybridize to a target site. For example, at least 1, at least 2, at least 3, at least 4, at least 5, at least 6, at least 7, or at least 8 mismatches (or more) between a target site and a non-coding RNA may be tolerated. A “target site” also refers to the location of a polynucleotide sequence within a plant genome that is bound and cleaved by any other site-specific nuclease that may not be guided by a non-coding RNA molecule, such as a zinc finger nuclease (ZFN), a transcription activator-like effector nuclease (TALEN), a meganuclease, etc., to introduce a DSB or single-stranded nick into the polynucleotide sequence and/or its complementary DNA strand. As used herein, a “site-specific nuclease” includes any zinc finger nuclease (ZFN), transcription activator-like effector nuclease (TALEN), ribonucleoprotein, meganuclease, recombinase, transposase, or other nuclease that can introduce a double-stranded break (DSB) or single-stranded nick into a polynucleotide sequence at or near a target site, such as a target site within the genome of a plant cell. As used herein, a “target region” or a “targeted region” refers to a polynucleotide sequence or region that is flanked by two or more target sites. Without being limiting, in some embodiments, a target region may be subjected to a mutation, deletion, insertion, or inversion. As used herein, “flanked” when used to describe a target region of a polynucleotide sequence or molecule, refers to two or more target sites of the polynucleotide sequence or molecule surrounding the target region, with one target site on each side of the target region.

As used herein, a “targeted genome editing technique” refers to any method, protocol, or technique that allows the precise and/or targeted editing of a specific location in a genome of a plant (i.e., the editing is largely or completely non-random) using a site-specific nuclease, such as a meganuclease, a zinc-finger nuclease (ZFN), an RNA-guided endonuclease (e.g., the CRISPR/Cas9 system or CRISPR/Cas12a system), a TALE (transcription activator-like effector)-endonuclease (TALEN), a recombinase, or a transposase. As used herein, “editing” or “genome editing” refers to generating a targeted mutation, deletion, insertion, inversion, or substitution of at least 1, at least 2, at least 3, at least 4, at least 5, at least 6, at least 7, at least 8, at least 9, at least 10, at least 15, at least 20, at least 25, at least 30, at least 35, at least 40, at least 45, at least 50, at least 75, at least 100, at least 250, at least 500, at least 1000, at least 2500, at least 5000, at least 10,000, or at least 25,000 nucleotides of an endogenous plant genome nucleic acid sequence. As used herein, “editing” or “genome editing” may also encompass the targeted insertion or site-directed integration of at least 1, at least 2, at least 3, at least 4, at least 5, at least 6, at least 7, at least 8, at least 9, at least 10, at least 15, at least 20, at least 25, at least 30, at least 35, at least 40, at least 45, at least 50, at least 75, at least 100, at least 250, at least 500, at least 750, at least 1000, at least 1500, at least 2000, at least 2500, at least 3000, at least 4000, at least 5000, at least 10,000, or at least 25,000 nucleotides into the endogenous genome of a plant. An “edit” or “genomic edit” in the singular refers to one such targeted mutation, deletion, inversion, substitution, or insertion, whereas “edits” or “genomic edits” refers to two or more targeted mutation(s), deletion(s), inversion(s), substitution(s) and/or insertion(s), with each “edit” being introduced via a targeted genome editing technique.

According to some embodiments, a site-specific nuclease may be co-delivered with a donor template molecule to serve as a template for making a desired edit, mutation, or insertion into the genome at the desired target site through repair of the double strand break (DSB) or nick created by the site-specific nuclease. According to some embodiments, a site-specific nuclease may be co-delivered with a DNA molecule comprising a selectable or screenable marker gene.

A site-specific nuclease provided herein may be selected from the group consisting of a zinc-finger nuclease (ZFN), a TALE-endonuclease (TALEN), a meganuclease, an RNA-guided endonuclease, a recombinase, a transposase, or any combination thereof. See, e.g., Khandagale et al. (Plant Biotechnol Rep 10:327-343, 2016); and Gaj et al. (Trends Biotechnol. 31(7):397-405, 2013. Zinc finger nucleases (ZFN) are synthetic proteins consisting of an engineered zinc finger DNA-binding domain fused to a cleavage domain (or a cleavage half-domain), which may be derived from a restriction endonuclease (e.g., FokI). The DNA binding domain may be canonical (C2H2) or non-canonical (e.g., C3H or C4). The DNA-binding domain can comprise one or more zinc fingers (e.g., 2, 3, 4, 5, 6, 7, 8, 9 or more zinc fingers) depending on the target site but may typically be composed of 3-4 (or more) zinc-fingers. Multiple zinc fingers in a DNA-binding domain may be separated by linker sequence(s). ZFNs can be designed to cleave almost any stretch of double-stranded DNA by modification of the zinc finger DNA-binding domain. ZFNs form dimers from monomers composed of a non-specific DNA cleavage domain (e.g., derived from the FokI nuclease) fused to a DNA-binding domain comprising a zinc finger array engineered to bind a target site DNA sequence. The amino acids at positions −1, +2, +3, and +6 relative to the start of the zinc finger α-helix, which contribute to site-specific binding to the target site, can be changed and customized to fit specific target sequences. The other amino acids may form a consensus backbone to generate ZFNs with different sequence specificities.

Methods and rules for designing ZFNs for targeting and binding to specific target sequences are known in the art. See, e.g., U.S. Patent App. Pub. Nos. 2005/0064474, 2009/0117617, and 2012/0142062. The FokI nuclease domain may require dimerization to cleave DNA and therefore two ZFNs with their C-terminal regions are needed to bind opposite DNA strands of the cleavage site (separated by 5-7 bp). The ZFN monomer can cut the target site if the two-ZF-binding sites are palindromic. A ZFN, as used herein, is broad and includes a monomeric ZFN that can cleave double stranded DNA without assistance from another ZFN. The term ZFN may also be used to refer to one or both members of a pair of ZFNs that are engineered to work together to cleave DNA at the same site. Because the DNA-binding specificities of zinc finger domains can be re-engineered using one of various methods, customized ZFNs can theoretically be constructed to target nearly any target sequence (e.g., at or near a gene in a plant genome). Publicly available methods for engineering zinc finger domains include Context-dependent Assembly (CoDA), Oligomerized Pool Engineering (OPEN), and Modular Assembly.

Transcription activator-like effectors (TALEs) can be engineered to bind practically any DNA sequence, such as at or near the genomic locus of a gene in a plant. TALE has a central DNA-binding domain composed of 13-28 repeat monomers of 33-34 amino acids. The amino acids of each monomer are highly conserved, except for hypervariable amino acid residues at positions 12 and 13. The two variable amino acids are called repeat-variable diresidues (RVDs). The amino acid pairs NI, NG, HD, and NN of RVDs preferentially recognize adenine, thymine, cytosine, and guanine/adenine, respectively, and modulation of RVDs can recognize consecutive DNA bases. This simple relationship between amino acid sequence and DNA recognition has allowed for the engineering of specific DNA binding domains by selecting a combination of repeat segments containing the appropriate RVDs.

TALENs are artificial restriction enzymes generated by fusing the TALE DNA binding domain to a nuclease domain. In some aspects, the nuclease is selected from a group consisting of PvuII, MutH, TevI, FokI, AlwI, MlyI, Sbfl, SdaI, StsI, CleDORF, Clo051, and Pept071. When each member of a TALEN pair binds to the DNA sites flanking a target site, the FokI monomers dimerize and cause a double-stranded DNA break at the target site. The term TALEN, as used herein, is broad and includes a monomeric TALEN that can cleave double stranded DNA without assistance from another TALEN. The term TALEN also refers to one or both members of a pair of TALENs that work together to cleave DNA at the same site.

Besides the wild-type FokI cleavage domain, variants of the FokI cleavage domain with mutations have been designed to improve cleavage specificity and cleavage activity. The FokI domain functions as a dimer, requiring two constructs with unique DNA binding domains for sites in the target genome with proper orientation and spacing. Both the number of amino acid residues between the TALEN DNA binding domain and the FokI cleavage domain and the number of bases between the two individual TALEN binding sites are parameters for achieving high levels of activity. PvuII, MutH, and TevI cleavage domains are useful alternatives to FokI and FokI variants for use with TALEs. PvuII functions as a highly specific cleavage domain when coupled to a TALE (see Yank et al., PLoS One 8:e82539, 2013). MutH is capable of introducing strand-specific nicks in DNA (see Gabsalilow et al., Nucleic Acids Research. 41:e83, 2013). TevI introduces double-stranded breaks in DNA at targeted sites (see Beurdeley et al., Nature Communications 4:1762, 2013).

The relationship between amino acid sequence and DNA recognition of the TALE binding domain allows for designable proteins. Software programs such as DNAWorks can be used to design TALE constructs. Other methods of designing TALE constructs are known to those of skill in the art. See Doyle et al. (Nucleic Acids Research 40:W117-122, 2012); Cermak et al. (Nucleic Acids Research 39:e82, 2011); and tale-nt.cac.cornell.edu/about. In another aspect, a TALEN provided herein is capable of generating a targeted DSB.

A site-specific nuclease may be a meganuclease. Meganucleases, which are commonly identified in microbes, such as the LAGLIDADG family of homing endonucleases, are unique enzymes with high activity and long recognition sequences (>14 bp) resulting in site-specific digestion of target DNA. Engineered versions of naturally occurring meganucleases typically have extended DNA recognition sequences (for example, 14 to 40 bp). The engineering of meganucleases can be more challenging than ZFNs and TALENs because the DNA recognition and cleavage functions of meganucleases are intertwined in a single domain. Specialized methods of mutagenesis and high-throughput screening have been used to create novel meganuclease variants that recognize unique sequences and possess improved nuclease activity.

A site-specific nuclease may be an RNA-guided nuclease. According to some embodiments, an RNA-guided endonuclease may be selected from the group consisting of Cas1, Cas1B, Cas2, Cas3, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, Cas12a (also known as Cpf1), CasX, CasY, and homologs or modified versions of any thereof, as well as Argonaute (non-limiting examples of Argonaute proteins include Thermus thermophilus Argonaute (TtAgo), Pyrococcus furiosus Argonaute (PfAgo), Natronobacterium gregoryi Argonaute (NgAgo), and homologs or modified versions of any thereof). According to some embodiments, an RNA-guided endonuclease is a Cas9 or Cas12a enzyme. The RNA-guided nuclease may be delivered as a protein with or without a guide RNA, or the guide RNA may be complexed with the RNA-guided nuclease enzyme and delivered as a ribonucleoprotein (RNP).

For RNA-guided endonucleases, a guide RNA molecule may be further provided to direct the endonuclease to a target site in the genome of the plant via base-pairing or hybridization to cause a DSB or nick at or near the target site. The guide RNA may be transformed or introduced into a plant cell or tissue as a gRNA molecule, or as a recombinant DNA molecule, construct or vector comprising a transcribable DNA sequence encoding the guide RNA operably linked to a promoter. As understood in the art, a guide RNA may comprise, for example, a CRISPR RNA (crRNA), a tracrRNA, a single-chain guide RNA (sgRNA), or any other RNA molecule that may guide or direct an endonuclease to a specific target site in the genome. A prototypical CRISPR-associated protein, Cas9 from S. pyogenes, naturally binds two RNAs, a CRISPR RNA (crRNA) guide and a trans-acting CRISPR RNA (tracrRNA), to assemble a CRISPR ribonucleoprotein (crRNP). A “single-chain guide RNA” (or “sgRNA”) is an RNA molecule comprising a crRNA covalently linked a tracrRNA by a linker sequence, which may be expressed as a single RNA transcript or molecule. The guide RNA comprises a guide or targeting sequence (also referred to herein as a “spacer sequence”) that is identical or complementary to a target site within the plant genome, such as at or near a gene. The guide RNA is typically a non-coding RNA molecule that does not encode a protein. The guide sequence of the guide RNA may be at least 10 nucleotides in length, such as 12-40 nucleotides, 12-30 nucleotides, 12-20 nucleotides, 12-35 nucleotides, 12-30 nucleotides, 15-30 nucleotides, 17-30 nucleotides, or 17-25 nucleotides in length, or about 12, 13, 14, 15, 16, 17, 18, 19, 20, 21, 22, 23, 24, 25 or more nucleotides in length. The guide sequence may be at least 95%, at least 96%, at least 97%, at least 99% or 100% identical or complementary to at least 10, at least 11, at least 12, at least 13, at least 14, at least 15, at least 16, at least 17, at least 18, at least 19, at least 20, at least 21, at least 22, at least 23, at least 24, at least 25, or more consecutive nucleotides of a DNA sequence at the genomic target site.

As used herein, with respective to a given sequence, a “complement”, a “complementary sequence” and a “reverse complement” are used interchangeably. All three terms refer to the inversely complementary sequence of a nucleotide sequence, i.e., to a sequence complementary to a given sequence in reverse order of the nucleotides.

As used herein, the term “antisense” refers to DNA or RNA sequences that are complementary to a specific DNA or RNA sequence. Antisense RNA molecules are single-stranded nucleic acids which can combine with a sense RNA strand or sequence or mRNA to form duplexes due to complementarity of the sequences. The term “antisense strand” refers to a nucleic acid strand that is complementary to the “sense” strand. The “sense strand” of a gene or locus is the strand of DNA or RNA that has the same sequence as an RNA molecule transcribed from the gene or locus (with the exception of uracil in RNA and thymine in DNA).

A protospacer-adjacent motif (PAM) may be present in the genome immediately adjacent and upstream to the 5′ end of the genomic target site sequence complementary to the targeting sequence of the guide RNA—i.e., immediately downstream (3′) to the sense (+) strand of the genomic target site (relative to the targeting sequence of the guide RNA) as known in the art. See, e.g., Wu et al. (Quant Biol. 2(2):59-70, 2014). The genomic PAM sequence on the sense (+) strand adjacent to the target site (relative to the targeting sequence of the guide RNA) may comprise 5′-NGG-3′. However, the corresponding sequence of the guide RNA (i.e., immediately downstream (3′) to the targeting sequence of the guide RNA) may generally not be complementary to the genomic PAM sequence.

In some embodiments, a site-specific nuclease is a recombinase. Non-limiting examples of recombinases that may be used include a serine recombinase attached to a DNA recognition motif, a tyrosine recombinase attached to a DNA recognition motif, or any recombinase enzyme known in the art attached to a DNA recognition motif. In certain embodiments, the site-specific nuclease is a recombinase or transposase, which may be a DNA transposase or recombinase attached or fused to a DNA binding domain. Non-limiting examples of recombinases include a tyrosine recombinase selected from the group consisting of a Cre recombinase, a Gin recombinase, a Flp recombinase, and a Tnp1 recombinase attached to a DNA recognition motif provided herein. In one aspect of the present disclosure, a Cre recombinase or a Gin recombinase provided herein is tethered to a zinc-finger DNA-binding domain, a TALE DNA-binding domain, or a Cas9 nuclease. In another aspect, a serine recombinase selected from the group consisting of a PhiC31 integrase, an R4 integrase, and a TP-901 integrase may be attached to a DNA recognition motif provided herein. In yet another aspect, a DNA transposase selected from the group consisting of a TALE-piggyBac and TALE-Mutator may be attached to a DNA binding domain provided herein.

Several site-specific nucleases, such as recombinases, zinc finger nucleases (ZFNs), meganucleases, and TALENs, are not RNA-guided and instead rely on their protein structure to determine their target site for causing the DSB or nick, or they are fused, tethered, or attached to a DNA-binding protein domain or motif. The protein structure of the site-specific nuclease (or the fused/attached/tethered DNA binding domain) may target the site-specific nuclease to the target site. According to many of these embodiments, non-RNA-guided site-specific nucleases, such as recombinases, zinc finger nucleases (ZFNs), meganucleases, and TALENs, may be designed, engineered, and constructed according to known methods to target and bind to a target site at or near the genomic locus of an endogenous gene of a plant to create a DSB or nick at such a genomic locus. The DSB or nick created by the non-RNA-guided site-specific nuclease may lead to knockdown of gene expression via repair of the DSB or nick, which may result in a mutation or insertion of a sequence at the site of the DSB or nick through cellular repair mechanisms. Such cellular repair mechanism may be guided by a donor template molecule.

As used herein, a “donor molecule”, “donor template”, or “donor template molecule” (collectively a “donor template”), which may be a recombinant polynucleotide, DNA or RNA donor template or sequence, is defined as a nucleic acid molecule having a homologous nucleic acid template or sequence (e.g., homology sequence) and/or an insertion sequence for site-directed, targeted insertion or recombination into the genome of a plant cell via repair of a nick or DSB in the genome of a plant cell. A donor template may be a separate DNA molecule comprising one or more homologous sequence(s) and/or an insertion sequence for targeted integration, or a donor template may be a sequence portion (i.e., a donor template region) of a DNA molecule further comprising one or more other expression cassettes, genes/transgenes, and/or transcribable DNA sequences. A donor template may be an Agrobacterium Transfer-DNA (T-DNA) molecule comprising one or more other expression cassettes, genes/transgenes, and/or transcribable DNA sequences. For example, a “donor template” may be used for site-directed integration of a transgene or construct, or as a template to introduce a mutation, such as an insertion, deletion, substitution, etc., into a target site within the genome of a plant. A targeted genome editing technique provided herein may comprise the use of one or more, two or more, three or more, four or more, or five or more donor molecules or templates. A donor template provided herein may comprise at least one, at least two, at least three, at least four, at least five, at least six, at least seven, at least eight, at least nine, or at least ten gene(s) or transgene(s) and/or transcribable DNA sequence(s). Alternatively, a donor template may comprise no genes, transgenes, or transcribable DNA sequences.

Without being limiting, a gene/transgene or transcribable DNA sequence of a donor template may include, for example, an insecticidal resistance gene, an herbicide tolerance gene, a nitrogen use efficiency gene, a water use efficiency gene, a yield enhancing gene, a nutritional quality gene, a DNA binding gene, a selectable marker gene, an RNAi or suppression construct, a site-specific genome modification enzyme gene, a single guide RNA of a CRISPR/Cas9 system, a guide RNA of a CRISPR/Cas12a system, a geminivirus-based expression cassette, or a plant viral expression vector system. According to other embodiments, an insertion sequence of a donor template may comprise a protein encoding sequence or a transcribable DNA sequence that encodes a non-coding RNA molecule, which may target an endogenous gene for suppression. A donor template may comprise a promoter operably linked to a coding sequence, gene, or transcribable DNA sequence, such as a constitutive promoter, a tissue-specific or tissue-preferred promoter, a developmental stage promoter, or an inducible promoter. A donor template may comprise a leader, enhancer, promoter, transcriptional start site, 5′-UTR, one or more exon(s), one or more intron(s), transcriptional termination site, region, or sequence, 3′-UTR, and/or polyadenylation signal, which may each be operably linked to a coding sequence, gene (or transgene) or transcribable DNA sequence encoding a non-coding RNA, a guide RNA, an mRNA and/or protein. A donor template may be a single-stranded or double-stranded DNA or RNA molecule or plasmid.

An “insertion sequence” of a donor template is a sequence designed for targeted insertion into the genome of a plant cell, which may be of any suitable length. For example, the insertion sequence of a donor template may be between 2 and 50,000, between 2 and 10,000, between 2 and 5000, between 2 and 1000, between 2 and 500, between 2 and 250, between 2 and 100, between 2 and 50, between 2 and 30, between 15 and 50, between 15 and 100, between 15 and 500, between 15 and 1000, between 15 and 5000, between 18 and 30, between 18 and 26, between 20 and 26, between 20 and 50, between 20 and 100, between 20 and 250, between 20 and 500, between 20 and 1000, between 20 and 5000, between 20 and 10,000, between 50 and 250, between 50 and 500, between 50 and 1000, between 50 and 5000, between 50 and 10,000, between 100 and 250, between 100 and 500, between 100 and 1000, between 100 and 5000, between 100 and 10,000, between 250 and 500, between 250 and 1000, between 250 and 5000, or between 250 and 10,000 nucleotides or base pairs in length. A donor template may also have at least one homology sequence or homology arm, such as two homology arms, to direct the integration of a mutation or insertion sequence into a target site within the genome of a plant via homologous recombination, wherein the homology sequence or homology arm(s) are identical or complementary, or have a percent identity or percent complementarity, to a sequence at or near the target site within the genome of the plant. When a donor template comprises homology arm(s) and an insertion sequence, the homology arm(s) will flank or surround the insertion sequence of the donor template. Each homology arm may be at least 70%, at least 75%, at least 80%, at least 85%, at least 90%, at least 95%, at least 96%, at least 97%, at least 99% or 100% identical or complementary to at least 20, at least 25, at least 30, at least 35, at least 40, at least 45, at least 50, at least 60, at least 70, at least 80, at least 90, at least 100, at least 150, at least 200, at least 250, at least 500, at least 1000, at least 2500, or at least 5000 consecutive nucleotides of a target DNA sequence within the genome of a plant.

Any method known in the art for site-directed integration may be used with the present disclosure. In the presence of a donor template molecule with an insertion sequence, the DSB or nick can be repaired by homologous recombination between homology arm(s) of the donor template and the plant genome, or by non-homologous end joining (NHEJ), resulting in site-directed integration of the insertion sequence into the plant genome to create the targeted insertion event at the site of the DSB or nick. Thus, site-specific insertion or integration of a transgene, transcribable DNA sequence, construct, or sequence may be achieved if the transgene, transcribable DNA sequence, construct or sequence is located in the insertion sequence of the donor template.

The introduction of a DSB or nick may also be used to introduce targeted mutations in the genome of a plant. According to this approach, mutations, such as deletions, insertions, inversions, and/or substitutions may be introduced at a target site via imperfect repair of the DSB or nick to produce a knock-out or knock-down of a gene. Such mutations may be generated by imperfect repair of the targeted locus even without the use of a donor template molecule. A “knock-out” of a gene may be achieved by inducing a DSB or nick at or near the endogenous locus of the gene that results in non-expression of the protein or expression of a non-functional protein, whereas a “knock-down” of a gene may be achieved in a similar manner by inducing a DSB or nick at or near the endogenous locus of the gene that is repaired imperfectly at a site that does not affect the coding sequence of the gene in a manner that would eliminate the function of the encoded protein. For example, the site of the DSB or nick within the endogenous locus may be in the upstream or 5′ region of the gene (e.g., a promoter and/or enhancer sequence) to affect or reduce its level of expression.

Similarly, such targeted knock-out or knock-down mutations of a gene may be generated with a donor template molecule to direct a particular or desired mutation at or near the target site via repair of the DSB or nick. The donor template molecule may comprise a homologous sequence with or without an insertion sequence and comprising one or more mutations, such as one or more deletions, insertions, inversions and/or substitutions, relative to the targeted genomic sequence at or near the site of the DSB or nick. For example, targeted knock-out or knock-down mutations of a gene may be achieved by substituting, inserting, deleting, or inverting at least a portion of the gene, such as by introducing a frame shift or premature stop codon into the coding sequence of the gene or disrupting a promoter sequence or the sequence of another non-coding regulatory element of the gene. A deletion of a portion of a gene may also be introduced by generating DSBs or nicks at two target sites and causing a deletion of the intervening target region flanked by the target sites.

In further embodiments, genetic modification of a plant may comprise transformation of a plant, plant part, plant tissue or plant cell to insert a polynucleotide or DNA sequence or transgene into the genome of the plant, plant part, plant tissue or plant cell. Methods for transformation of plants that are known in the art and applicable to many crop species include, but are not limited to, electroporation, microprojectile or particle bombardment, microinjection, PEG-mediated transformation, Agrobacterium-mediated transformation, and other modes of direct DNA uptake. Bacteria known to mediate plant cell transformation include a number of species of bacterial genera, species, and strains that may be assigned to the order Rhizobiales other than Agrobacterium, including but not limited to, bacterial species and strains from the taxonomic families Rhizobiaceae (e.g. Rhizobium spp., Sinorhizobium spp.), Phyllobacteriaceae (e.g. Mesorhizobium spp., Phyllobacterium spp.), Brucellaceae (e.g. Ochrobactrum spp.), Bradyrhizobiaceae (e.g. Bradyrhizobium spp.), and Xanthobacteraceae (e.g. Azorhizobium spp.), among others. According to some embodiments, Agrobacterium-mediated transformation is mediated by Agrobacterium tumefaciens. Targets for such transformation have often been undifferentiated callus tissues, although differentiated tissues have also been used for transient and stable plant transformation. As is well known in the art, other methods for plant transformation may be utilized, for instance as described by Miki et al., (1993, “Procedures for Introducing Foreign DNA into Plants,” in Methods in Plant Molecular Biology and Biotechnology, Glick B. R. and Thompson, J. E. Eds., CRC Press, Inc., Boca Raton, pages 67-88).

In specific embodiments, microprojectile bombardment may be employed to deliver a polynucleotide or DNA molecule, vector, sequence, segment, or RNP to a cell. In this method, particles are coated with a polynucleotide or polynucleotide/protein complex and delivered into cells by a propelling force. Exemplary particles may include those comprised of tungsten, platinum, or gold. For bombardment, target cells may be arranged on a solid culture medium. The cells to be bombarded are positioned at an appropriate distance below the projectile stopping plate. A polynucleotide may be delivered into plant cells by acceleration using a biolistics particle delivery system, which may propel particles coated with a DNA or polynucleotide molecule through a screen, such as a stainless steel or Nytex screen, and toward the cells positioned on a surface. The screen may disperse the particles so that they are not delivered to the recipient cells in large aggregates. Microprojectile bombardment techniques are widely applicable and may be used to transform a variety of plant species.

Agrobacterium-mediated or Rhizobiales-mediated transformation of cells is another widely applicable system for introducing heterologous and/or recombinant DNA molecules into plant cells. Modern Agrobacterium-mediated transformation vectors are capable of replication in E. coli as well as in Agrobacterium, allowing for convenient manipulations (see, e.g., Klee et al., Nat. Biotechnol., 3(7):637-642, 1985). Moreover, recent technological advances in vectors for Agrobacterium-mediated gene transfer have improved the arrangement of genes and restriction sites in the vectors to facilitate the construction of vectors capable of expressing various polypeptide coding genes. The vectors described have convenient multi-linker regions flanked by a promoter and a polyadenylation site for direct expression of inserted polypeptide coding genes. Additionally, Agrobacterium containing both armed and disarmed Ti plasmids can be used for transformation. Agrobacterium-mediated transformation is often the method of choice for many plant species. The use of Agrobacterium-mediated plant integrating vectors to introduce DNA into plant cells is known in the art (see, e.g., Fraley et al., Nat. Biotechnol., 3:629-635, 1985; U.S. Pat. No. 5,563,055).

A number of promoters and expression elements have utility for plant gene expression of any selectable marker, scoreable marker, transgene, or any other gene of agronomic interest. Promoters may include any constitutive promoter, tissue-specific promoter, organ-specific promoter, tissue-preferred promoter, organ-preferred promoter, inducible promoter, reproductive tissue promoter, developmental stage promoter, viral promoter, or the like. Examples of various types of promoters and expression elements are known in art. Expression elements that may be useful for plant gene expression may include, for example, various promoters, enhancers, leaders, 5′ and 3′ untranslated regions, introns, terminators, and the like, as known in the art. A selectable or screenable marker or gene of interest may also be fused to a transit peptide or other targeting sequence. Transport of proteins produced by transgenes to a subcellular compartment such as the chloroplast, vacuole, peroxisome, glyoxysome, cell wall, nucleus, or mitochondrion or for secretion into the apoplast, may be accomplished by means of operably linking the nucleotide sequence encoding a signal or targeting sequence to the 5′ and/or 3′ region of a gene encoding the protein of interest. Targeting sequences at the 5′ and/or 3′ end of the structural gene may determine, during protein synthesis and processing, where the encoded protein is ultimately compartmentalized. The presence of a signal sequence directs a polypeptide to either an intracellular organelle or subcellular compartment or for secretion to the apoplast. Many signal sequences are known in the art. See, for example Becker et al. (Plant Mol. Biol., 20:49, 1992); Knox et al. (Plant Mol. Biol., 9:3-17, 1987); Lerner et al. (Plant Physiol., 91:124-129, 1989); Fontes et al. (Plant Cell, 3:483-496, 1991); Matsuoka et al. (Proc. Natl. Acad. Sci. USA, 88:834, 1991); Gould et al. (J. Cell. Biol., 108:1657, 1989); Creissen et al. (Plant J., 2:129, 1991); Kalderon et al. (Cell, 39:499-509, 1984); Steifel et al. (Plant Cell, 2:785-793, 1990).

Examples of constitutive promoters may include, for example, the cauliflower mosaic virus (CaMV) 35S promoter, which confers constitutive, high-level expression in most plant tissues (see, e.g., Odel et al., Nature, 313:810, 1985), including monocots (see, e.g., Dekeyser et al., Plant Cell, 2:591, 1990; Terada and Shimamoto, Mol. Gen. Genet., 220:389, 1990), a tandemly duplicated version of the CaMV 35S promoter, the enhanced 35S promoter (e35S), the nopaline synthase promoter (An et al., Plant Physiol., 88:547, 1988), the octopine synthase promoter (Fromm et al., Plant Cell, 1:977, 1989), and the figwort mosaic virus (FMV) promoter as described in U.S. Pat. No. 5,378,619 and an enhanced version of the FMV promoter (eFMV) where the promoter sequence of FMV is duplicated in tandem, the cauliflower mosaic virus 19S promoter, a sugarcane bacilliform virus promoter, a commelina yellow mottle virus promoter, and other plant DNA virus promoters known to express in plant cells. Constitutive promoters may also include promoters derived from plant genomes such as the maize ubiquitin promoter (Christensen et al., Plant Mol. Biol., 18: 675, 689, 1992), maize actin promoter (McElroy, et al., Plant Cell, 2:163-171, 1990), or Arabidopsis S-Adenosylmethionine synthetase promoter (see U.S. Pat. No. 8,809,628).

With an inducible promoter, the rate of transcription increases in response to an inducing agent or signal. Any inducible promoter can be used according to the embodiments of the present disclosure. A variety of plant gene promoters that are regulated in response to environmental, hormonal, chemical, and/or developmental signals can be used for expression of an operably linked gene in plant cells, including promoters regulated by (1) heat (e.g., Callis et al., Plant Physiol., 88:965, 1988), (2) light (e.g., pea rbcS-3A promoter, Kuhlemeier et al., Plant Cell, 1:471, 1989; maize rbcS promoter, Schaffner and Sheen, Plant Cell, 3:997, 1991; or chlorophyll a/b-binding protein promoter, Simpson et al., EMBO J., 4:2723, 1985), (3) hormones, such as abscisic acid (Marcotte et al., Plant Cell, 1:969, 1989), (4) wounding (e.g., Siebertz et al., Plant Cell, 1:961, 1989), or (5) chemicals such as methyl jasmonate, salicylic acid, or Safener. It may also be advantageous to employ organ-specific or tissue specific promoters known in the art (e.g., Roshal et al., EMBO J., 6:1155, 1987; Schernthaner et al., EMBO J., 7:1249, 1988; Bustos et al., Plant Cell, 1:839, 1989).

Exemplary polynucleotide or DNA molecules which may be introduced include, for example, DNA sequences or genes from another species, or even genes or sequences which originate with or are present in the same species, but are incorporated into recipient cells by genetic engineering methods rather than classical reproduction or breeding techniques. However, the term “exogenous” is also intended to refer to genes that are not normally present in the cell being transformed, or perhaps not present in the form, structure, or location. A polynucleotide may include a DNA molecule or sequence which is already present in the plant cell, is from another plant, is from a different organism, is exogenous or generated externally. A transgene or expression cassette may encode a mRNA and protein or an RNA molecule for suppression, such as a miRNA, siRNA, dsRNA, antisense RNA, inverted repeat RNA, or the like. A polynucleotide may be an exogenous, heterologous and/or recombinant polynucleotide or DNA molecule or sequence.

Many different transgenes or genetic modifications could potentially be introduced according to the present disclosure, which may provide a beneficial agronomic trait of a crop plant having the transgene or modification. In particular embodiments, the transgene or genetic modification introduced into the genome of a plant cell may comprise or encode a sequence having at least about 70%, about 75%, about 80%, about 85%, about 90%, about 91%, about 92%, about 93%, about 94%, about 95%, about 96%, about 97%, about 98%, about 99%, or 100% sequence identity to any one of SEQ ID NOs:1-36, or fragments thereof, including any range derivable therebetween. In some embodiments, the transgene or genetic modification introduced into the genome of a plant cell may confer icafolin herbicide tolerance.

Additional non-limiting examples of particular transgenes or genetic modifications and corresponding phenotypes one may choose to introduce into a cell include one or more genes for insect resistance, such as a Bacillus thuringiensis (B.t.) gene, pest tolerance, such as genes for fungal disease control, herbicide tolerance, such as genes conferring glyphosate tolerance, and genes for quality improvements, such as yield, nutritional enhancements, environmental or stress tolerances, or any desirable changes in plant physiology, growth, development, morphology or plant product(s). For example, structural genes would include any gene that confers insect resistance, including, but not limited to, a Bacillus insect control protein gene as described in WO 99/31248, herein incorporated by reference in its entirety, U.S. Pat. No. 5,689,052, herein incorporated by reference in its entirety, U.S. Pat. Nos. 5,500,365 and 5,880,275, herein incorporated by reference in their entirety. In some embodiments, the structural gene can confer tolerance to the herbicide glyphosate as conferred by genes including, but not limited to, Agrobacterium strain CP4 glyphosate resistant EPSPS gene (aroA:CP4) as described in U.S. Pat. No. 5,633,435, herein incorporated by reference in its entirety, or glyphosate oxidoreductase gene (GOX) as described in U.S. Pat. No. 5,463,175, herein incorporated by reference in its entirety.

A variety of assays are known in the art and may be used to confirm the presence of a genetic modification, exogenous DNA sequence, genetic marker, expression cassette or transgene in transformed, edited, or genetically modified cells, plants, or plant parts. Methods and techniques are provided for screening for, and/or identifying, cells or plants, etc., for the presence of targeted edits or transgenes, and selecting cells or plants comprising targeted edits or transgenes, which may be based on one or more phenotypes or traits, or on the presence or absence of a molecular marker or polynucleotide or protein sequence in the cells or plants. As used herein, a “molecular technique” refers to any method known in the fields of molecular biology, biochemistry, genetics, plant biology, or biophysics that involves the use, manipulation, or analysis of a nucleic acid, a protein, or a lipid. Without being limiting, molecular techniques useful for detecting the presence of a modified sequence in a genome include phenotypic screening; molecular marker technologies such as SNP analysis by TaqMan® or Illumina/Infinium technology; Southern blot hybridization; PCR; enzyme-linked immunosorbent assay (ELISA); and sequencing (e.g., Sanger, Illumina®, 454, Pac-Bio, Ion Torrent™). In one aspect, a method of detection provided herein comprises phenotypic screening. In another aspect, a method of detection provided herein comprises SNP analysis. In a further aspect, a method of detection provided herein comprises a Southern blot hybridization. In a further aspect, a method of detection provided herein comprises PCR. In an aspect, a method of detection provided herein comprises ELISA. In a further aspect, a method of detection provided herein comprises determining the sequence of a nucleic acid or a protein. Without being limiting, nucleic acids can be detected using hybridization. Hybridization between nucleic acids is discussed in detail in Sambrook et al. (1989, Molecular Cloning: A Laboratory Manual, 2nd Ed., Cold Spring Harbor Laboratory Press, Cold Spring Harbor, NY).

Nucleic acid or polynucleotide molecules can be isolated using techniques routine in the art. For example, nucleic acids can be isolated using any method including, without limitation, recombinant nucleic acid technology and/or PCR. General PCR techniques are described, for example in PCR Primer: A Laboratory Manual, Dieffenbach & Dveksler, Eds., Cold Spring Harbor Laboratory Press, 1995. Recombinant nucleic acid techniques include, for example, restriction enzyme digestion and ligation, which can be used to isolate a nucleic acid. Isolated nucleic acids also can be chemically synthesized, either as a single nucleic acid molecule or as a series of oligonucleotides.

Detection (e.g., of an amplification product, of a hybridization complex, of a polypeptide) can be accomplished using detectable labels that may be attached or associated with a hybridization probe or antibody. The term “label” is intended to encompass the use of direct labels as well as indirect labels. Detectable labels include enzymes, prosthetic groups, fluorescent materials, luminescent materials, bioluminescent materials, and radioactive materials. The screening and selection of modified (e.g., edited) plants or plant cells can be through any methodologies known to those skilled in the art of molecular biology. Examples of screening and selection methodologies include, but are not limited to, Southern analysis, PCR amplification for detection of a polynucleotide, Northern blot hybridization, RNase protection, primer-extension, RT-PCR amplification for detecting RNA transcripts, Sanger sequencing, Next Generation sequencing technologies (e.g., Illumina®, PacBio®, Ion Torrent™, etc.) enzymatic assays for detecting enzyme or ribozyme activity of polypeptides and polynucleotides, and protein gel electrophoresis, Western blot analysis, immunoprecipitation, and enzyme-linked immunoassays to detect polypeptides. Other techniques such as in situ hybridization, enzyme staining, and immunostaining also can be used to detect the presence or expression of polypeptides and/or polynucleotides. Methods for performing all of the referenced techniques are known in the art.

As used herein, the term “polypeptide” refers to a chain of at least two covalently linked amino acids. Polypeptides can be encoded by polynucleotides provided herein. An example of a polypeptide is a protein. Proteins provided herein can be encoded by nucleic acid molecules provided herein. Polypeptides can be purified from natural sources (e.g., a biological sample) by known methods such as DEAE ion exchange, gel filtration, and hydroxyapatite chromatography. A polypeptide also can be purified, for example, by expressing a nucleic acid in an expression vector. In addition, a purified polypeptide can be obtained by chemical synthesis. The extent of purity of a polypeptide can be measured using any appropriate method, e.g., column chromatography, polyacrylamide gel electrophoresis, or HPLC analysis.

Polypeptides can be detected using antibodies. Techniques for detecting polypeptides using antibodies include enzyme linked immunosorbent assays (ELISAs), Western blots, immunoprecipitations, and immunofluorescence. An antibody provided herein can be a polyclonal antibody or a monoclonal antibody. An antibody having specific binding affinity for a polypeptide provided herein can be generated using methods well known in the art. An antibody provided herein can be attached to a solid support such as a microtiter plate using methods known in the art.

### D. Genetically Modified Plants and Herbicide Tolerance

One aspect of the present disclosure includes genetically modified plant cells, plant tissues, plants, and seeds that comprise the recombinant DNA molecules or engineered proteins provided by the present disclosure. These cells, tissues, plants, and seeds, in certain embodiments, exhibit herbicide tolerance to at least one icafolin herbicide. In one embodiment, these cells, tissues, plants, and seeds may optionally exhibit tolerance to a least one additional herbicide.

Icafolin is a novel post-emergence isoxazole herbicide that acts on dicot (broadleaf) and monocot (narrow leaf/grass) weeds. Non-limiting examples of icafolin herbicides include methyl (2R*,4R*)-4-[[(5S)-3-(3,5-difluorophenyl)-5-vinyl-4H-isoxazole-5-carbonyl]amino]tetrahydrofuran-2-carboxylate; methyl (2R,4R)-4-[[(5S)-3-(3,5-difluorophenyl)-5-vinyl-4H-isoxazole-5-carbonyl]-amino]tetrahydrofuran-2-carboxylate; methyl (2S,4S)-4-[[(5S)-3-(3,5-difluorophenyl)-5-vinyl-4H-isoxazole-5-carbonyl]amino]tetrahydrofuran-2-carboxylate; (2R*,4R*)-4-[[(5S)-3-(3,5-difluorophenyl)-5-vinyl-4H-isoxazole-5-carbonyl]amino]tetrahydrofuran-2-carboxylic acid; (2R,4R)-4-[[(5S)-3-(3,5-difluorophenyl)-5-vinyl-4H-isoxazole-5-carbonyl]amino]tetrahydrofuran-2-carboxylic acid; (2S,4S)-4-[[(5S)-3-(3,5-difluorophenyl)-5-vinyl-4H-isoxazole-5-carbonyl]amino]tetrahydrofuran-2-carboxylic acid, and combinations of any thereof. In certain embodiments, cells, seeds, plants, and plant parts provided by the present disclosure exhibit herbicide tolerance to at least one icafolin herbicide.

Icafolin may be applied to a plant growth area comprising the plants and seeds provided by the present disclosure as a method for controlling weeds. Alternatively, or in addition, icafolin may be applied to a plant growth area prior to planting of the seeds provided by the present disclosure. In many embodiments, plants and seeds provided by the present disclosure comprise an herbicide tolerance trait and as such are tolerant to the application of at least one icafolin herbicide. The herbicide application rate of an icafolin herbicide may vary with external conditions, including, but not limited to temperature, humidity and the particular icafolin herbicide used. In a particular embodiment, the icafolin herbicide application may be the recommended commercial rate (1×) or any fraction or multiple thereof, such as half of the recommended commercial field rate (0.5×) or twice the recommended commercial rate (2×). Herbicide rates may be expressed as acid equivalent per pound per acre (lb ae/acre) or acid equivalent per gram per hectare (g ae/ha) or as pounds active ingredient per acre (lb ai/acre) or grams active ingredient per hectare (g ai/ha), depending on the herbicide and the formulation. In some embodiments, the application rate of an icafolin herbicide may be about 0.001 kg/ha to about 1.0 kg/ha or more of active substance, about 0.005 kg/ha to about 750 kg/ha of active substance, or about 5 g/ha to about 250 g/ha of active substance. The 1× label rate for icafolin herbicides is about 5 g/ha to about 250 g/ha of active substance. When a mixture of the (2R, 4R)-isomer and the (2S, 4S)-isomer is used, the isomers can be present in the mixture in a ratio of about 1.2:0.8 to about 0.8:1.2, preferably in a ratio of about 1.1:0.9 to about 0.9:1:1 (e.g., the isomers can be present in a about a 1:1 ratio). The plant growth area may or may not comprise weed plants at the time of herbicide application. A herbicidally effective dose of an icafolin herbicide for use in an area for controlling weeds may be about 0.1× to about 30× label rate over a growing season. One (1) acre is equivalent to 2.47105 hectares and one (1) pound is equivalent to 453.592 grams. Herbicide application rates can be converted between English and metric as: (lb ai/ac) multiplied by 1.12=(kg ai/ha) and (kg ai/ha) multiplied by 0.89=(lb ai/ac).

Application of one or more herbicides may be performed sequentially or tank mixed with a combination of one or more herbicides. Multiple applications of one herbicide or of two or more herbicides, in combination or alone, may be used over a growing season to areas comprising transgenic plants of the present disclosure for the control of a broad spectrum of dicot weeds, monocot weeds, or both. In some embodiments, two applications of herbicide (such as a pre-planting application and a post-emergence application or a pre-emergence application and a post-emergence application) or three applications (such as a pre-planting application, a pre-emergence application, and a post-emergence application or a pre-emergence application and two post-emergence applications) may be applied.

As used herein, “tolerance” or “herbicide tolerance” or “herbicide tolerance activity” refers to a plant, seed, or cell's ability to resist the toxic effects of an herbicide when applied. Herbicide tolerant crops can continue to grow and are unaffected or minimally affected by the presence of the applied chemical. As used herein, an “herbicide tolerance trait” refers to a transgenic trait imparting improved herbicide tolerance to a plant, plant part, seed, or cell as compared to a wild-type plant. Non-limiting examples of plants which may be produced with an herbicide tolerance trait of the present disclosure include soybean plants, maize plants, cotton plants, barley plants, sorghum plants, rice plants, wheat plants, sugar cane plants, sugar beet plants, alfalfa plants, Brassica plants, and vegetable plants, and fruit plants (e.g., onion, tomato, pepper, curcurbit, pea, broccoli, cabbage, carrot, artichoke, lettuce, radish, spinach, cauliflower, zucchini, leek, potato, Brussels sprouts, tomatillo, beans, okra, apple, pear, cherry, peach, apricot, plum, banana, plantain, grape, citrus, avocado, mango, berry, etc.).

In certain aspects, the genetically modified plants, progeny, seeds, plant cells, and plant parts of the present disclosure may also contain one or more additional traits. Additional traits may be introduced by crossing a plant containing a recombinant DNA molecule provided by the disclosure with another plant containing one or more additional trait(s). As used herein, “crossing” refers to breeding two individual plants to produce a progeny plant. Two plants may thus be crossed to produce progeny that contain desirable traits from each parent. As used herein “progeny” refers the offspring of any generation of a parent plant, and transgenic progeny comprise a polynucleotide molecule provided by the present disclosure, inherited from at least one parent plant. Additional trait(s) also may be introduced by co-transforming a polynucleotide molecule for the additional transgenic trait(s) with a polynucleotide molecule comprising a recombinant polynucleotide molecule provided by the present disclosure, or by inserting the additional trait(s) into a transgenic plant comprising a polynucleotide molecule of the present disclosure or vice versa (for example, by using any of the methods of plant transformation or genome editing). Such additional traits include, but are not limited to, increased insect resistance, increased disease resistance (e.g., to Asian soybean rust), increased water use efficiency, increased yield performance, increased drought resistance, increased seed quality, improved nutritional quality, hybrid seed production, and herbicide tolerance, in which the trait is measured with respect to a wild-type plant. Exemplary additional herbicide tolerance traits may include transgenic or non-transgenic tolerance to one or more herbicides such as ACCase inhibitors (for example aryloxyphenoxy propionates and cyclohexanediones), ALS inhibitors (for example sulfonylureas, imidazolinones, triazoloyrimidines, and triazolinones) EPSPS inhibitors (for example glyphosate), synthetic auxins (for example phenoxys, benzoic acids, carboxylic acids, semicarbazones), photosynthesis inhibitors (for example triazines, triazinones, nitriles, benzothiadiazoles, and ureas), glutamine synthesis inhibitors (for example glufosinate), HPPD inhibitors (for example isoxazoles, pyrazolones, and triketones), PPO inhibitors (for example diphenylethers, N-phenylphthalimide, aryl triazinones, and pyrimidinediones), PDS inhibitors (for example, amides, pyridiazinones, and pyridines), and long-chain fatty acid inhibitors (for example chloroacetamindes, oxyacetamides, and pyrazoles), among others. Exemplary insect resistance traits may include resistance to one or more insect members within one or more of the orders of Lepidoptera, Coleoptera, Hemiptera, and Homoptera, among others. Such additional traits are well known to one of skill in the art; for example, and a list of such transgenic traits is provided by the United States Department of Agriculture's (USDA) Animal and Plant Health Inspection Service (APHIS).

A cell transformed with a polynucleotide molecule of the present disclosure may, in certain embodiments, be selected for the presence of the polynucleotide molecule or its encoded polypeptide before or after regenerating such a cell into a transgenic plant. In some embodiments, transgenic plants comprising such a polynucleotide may thus be selected by identifying a transgenic plant that comprises the polynucleotide molecule or the encoded polypeptide, and/or displays an altered trait relative to an otherwise isogenic control plant. Such a trait may be, for example, tolerance to an icafolin herbicide.

Transgenic plants and progeny that contain a transgenic trait provided by the disclosure may be used according to any breeding method known in the art. In plant lines comprising two or more transgenic traits, the transgenic traits may be independently segregating, linked, or a combination of both in plant lines comprising three or more transgenic traits. Back-crossing to a parental plant and out-crossing with a non-transgenic plant are also contemplated by the present disclosure, as is vegetative propagation. Descriptions of breeding methods that are used for different traits and crops are well known to those of skill in the art. To confirm the presence of the transgene(s) in a particular plant or seed, a variety of assays may be performed. Such assays include, for example, molecular biology assays, such as Southern and northern blotting, PCR, and DNA sequencing; biochemical assays, such as detecting the presence of a protein product, for example, by immunological means (ELISAs and western blots) or by enzymatic function; plant part assays, such as leaf or root assays; and also, by analyzing the phenotype of the whole plant.

Introgression of a transgenic trait into a plant genotype is achieved as the result of the process of backcross conversion. A plant genotype into which a transgenic trait has been introgressed may be referred to as a backcross converted genotype, line, inbred, or hybrid. Similarly, a plant genotype lacking the desired transgenic trait may be referred to as an unconverted genotype, line, inbred, or hybrid.

The term “about” is used to indicate that a value includes the standard deviation of the mean for the device or method being employed to determine the value. The use of the term “or” in the claims is used to mean “and/or” unless explicitly indicated to refer to alternatives only or the alternatives are mutually exclusive. When used in conjunction with the word “comprising” or other open language in the claims, the words “a” and “an” denote “one or more,” unless specifically noted otherwise. The terms “comprise,” “have,” and “include” are open-ended linking verbs. Any forms or tenses of one or more of these verbs, such as “comprises,” “comprising,” “has,” “having,” “includes,” and “including,” are also open-ended. For example, any method that “comprises,” “has,” or “includes” one or more steps is not limited to possessing only those one or more steps and also covers other unlisted steps. Similarly, any system or method that “comprises,” “has,” or “includes” one or more components is not limited to possessing only those components and covers other unlisted components. As used herein, the term “consists essentially of”, when used in reference to a nucleotide or amino acid sequence of the present disclosure, means that the nucleotide sequence or amino acid sequence, may contain additional nucleotides or amino acids so long as the additional nucleotides or amino acids do not materially alter the function of the recited sequences. The term “materially alter,” as applied to a nucleotide sequence or amino acid sequence of the present disclosure, refers to a decrease in herbicide tolerance of the encoded polypeptide of at least 25%. For example, additional nucleotides or amino acids added to a nucleotide or an amino acid sequence of the present disclosure may be deemed to “materially alter” the encoded polypeptide or polypeptide sequence, if such additions decrease the conferred herbicide tolerance by at least 25%.

Other objects, features, and advantages of the present disclosure are apparent from detailed description provided herein. It should be understood, however, that the detailed description and any specific examples provided, while indicating specific embodiments of the disclosure, are given by way of illustration only, since various changes and modifications within the spirit and scope of the disclosure will become apparent to those skilled in the art from this detailed description. Any embodiment of the present disclosure may be used in combination with any other embodiment described herein.

All references herein are incorporated herein by reference in their entirety.

### EXAMPLES

### Example 1: Icafolin Herbicide Tolerance Evaluation of Soybean Plants Expressing CYP Proteins

This example describes testing the ability of different cytochrome P450 genes to confer tolerance to icafolin herbicide to transgenic plants.

A transgenic plant-based screening approach was employed to identify enzymes capable of deactivating icafolin. One of the gene families explored was the cytochrome P450 (CYP) superfamily. Forty-one CYP proteins were screened for tolerance to icafolin, including two CYP72A family members, CYP12 (SEQ ID NO:1) and CYP13 (SEQ ID NO:2), and two CYP81E family members, CYP14 (SEQ ID NO:3) and CYP15 (SEQ ID NO:4), previously identified from a population of Palmer pigweed (Amaranthus palmeri). Gene expression profiling experiments in populations of waterhemp (Amaranthus tuberculatus) tolerant to the herbicides 4-hydroxyphenylpyruvate dioxygenase (HPPD) inhibitor and 2,4-D amine, previously indicated that CYP81E genes are significantly upregulated following 2,4-D treatment. See, PCT Publication No. WO2022/051340. The CYP14 and CYP15 proteins share 98.98% sequence identity, differing by 5 amino acids.

Prior to plant transformation, the Palmer pigweed nucleotide sequences encoding the CYP enzymes were codon optimized for dicot expression. CYP12_var, a variant of CYP12 protein, comprising a serine amino acid inserted at position 2 was also generated and screened for tolerance to icafolin herbicides. Table 2 provides the sequence identifiers (SEQ ID NOs) corresponding to the protein and nucleotide sequences tested.

TABLE 2
Amaranthus palmeri Cytochrome p450 Genes.

| | | | Amaranthus | Dicot codon |
|---|---|---|---|---|
| Cytochrome | CYP | Protein | palmeri DNA | optimized DNA |
| P450 gene | Subfamily | Sequence | sequence | sequence |
| CYP12_var | CYP72A | SEQ ID NO: 5 | N/A | SEQ ID NO: 10 |
| CYP13 | CYP72A | SEQ ID NO: 2 | SEQ ID NO: 7 | SEQ ID NO: 11 |
| CYP14 | CYP81E | SEQ ID NO: 3 | SEQ ID NO: 8 | SEQ ID NO: 12 |
| CYP15 | CYP81E | SEQ ID NO: 4 | SEQ ID NO: 9 | SEQ ID NO: 13 |

The codon-optimized polynucleotide sequences were placed into plant transformation cassettes with each CYP coding sequence operably-linked to a constitutive expression element comprising the promoter, leader, and intron sequence of Arabidopsis thaliana Ubiquitin 3 (SEQ ID NO:14) and a Medicago truncatula photosystem II terminator (SEQ ID NO:15). Four plant transformation vectors were created, each comprising one of the CYP gene expression cassettes and an aadA selectable marker expression cassette, conferring resistance to the antibiotics spectinomycin and streptomycin. Soybean cultivar AG3555 seed-derived embryo explants were transformed with the vectors by Agrobacterium-mediated transformation using known methods in the art, and transformed plantlets were selected based on growth in the presence of spectinomycin. Plantlets comprising single copy events were grown in the greenhouse to produce R₁ seeds. R₁ plants were subsequently selected for homozygous CYP-positive plants, either by molecular analysis of young leaf tissue or by seed chipping prior to planting. These selected plants were then screened for tolerance to icafolin herbicide.

Herbicide tolerance screens were performed by selecting R₁ plants at roughly the V2-V3 developmental stage and applying icafolin herbicide with a track sprayer under standard greenhouse conditions. Two independent transgenic events per transgene were screened. The herbicide was formulated with 1% v/v methylated seed oil (MSO) and 2.5% v/v ammonium sulfate (AMS) and sprayed with a XR nozzle at 15 gallons per hectare (GPA) to mimic field conditions. The icafolin used in this Example and in the experiments described in Examples 2-4 was a mixture of the methyl ester forms of the 2R, 4R and 2S, 4S isomers. The icafolin formulation was sprayed at two rates: 5 g active ingredient (ai)/hectare (ha) and 25 g ai/ha. The herbicide treated soybean plants were returned to the greenhouse following spray application and allowed to grow under standard conditions. Plants were rated for herbicide injury at approximately 14 and 21 days after treatment (DAT). Untreated transgenic plants and control (wild-type) plants were used for phenotypic comparison. The herbicide injury rating accounts for plant malformation, stunting, chlorosis, necrosis, and plant death relative to the untreated control plants. An injury rating of approximately 90% or above indicates nearly complete plant death. An injury rating of less than 30% indicates partial tolerance, and an injury rating of less than 20% indicates good tolerance. Injury ratings were collected and analyzed, and photographs of representative plants were taken. The injury ratings are summarized in Table 3.

TABLE 3
Injury Ratings (in %) Following Application of
Icafolin Herbicide in Soybean.
Experiment 1

| 5 g ai/ha icafolin treatment | | 25 g ai/ha icafolin treatment | |
|---|---|---|---|
| Percent | Percent | Percent | Percent |
| Injury | injury | Injury | injury |
| 15 DAT | 21 DAT | 15 DAT | 21 DAT |

CYP12_var

| | | | | |
|---|---|---|---|---|
| Event 909 | 39 | 50 | 57 | 77 |
| Event 932 | 42 | 47 | 69 | 80 |

CYP13

| | | | | |
|---|---|---|---|---|
| Event 943 | 42 | 59 | 74 | 80 |
| Event 982 | 41 | 52 | 69 | 80 |

CYP14

| | | | | |
|---|---|---|---|---|
| Event 267 | 14 | 12 | 39 | 47 |
| Event 309 | 33 | 36 | 71 | 81 |

CYP15

| | | | | |
|---|---|---|---|---|
| Event 784 | 8 | 3 | 28 | 32 |
| Event 794 | 8 | 6 | 36 | 41 |

Control

| | | | | |
|---|---|---|---|---|
| Control | 42 | 50 | 75 | 81 |

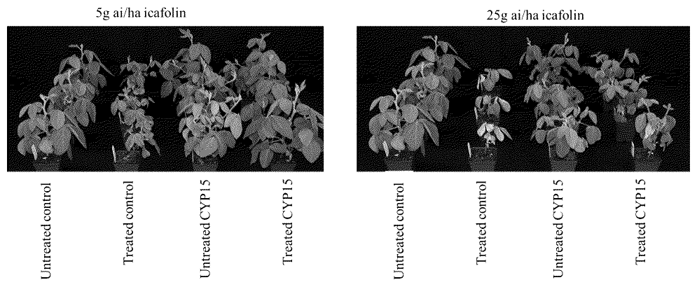

Treated control (wild-type) soybean was heavily injured at the 5 g ai/ha rate and nearly completely killed at the 25 g ai/ha rate. CYP14-expressing and CYP15-expressing transgenic plants showed unexpected tolerance to icafolin. The herbicide spray treatments were repeated a second time with two additional transgenic events generated from each of the CYP14 and CYP15 transformations. As shown in Table 4, the tolerance to icafolin was recapitulated in these events. FIG. 1 shows photographs of representative control and CYP 15-expressing transgenic plants following icafolin treatment.

TABLE 4
Injury Ratings (in %) Following Application of
Icafolin Herbicide in Soybean.
Experiment 2

| 5 g ai/ha icafolin treatment | | 25 g ai/ha icafolin treatment | |
|---|---|---|---|
| Percent | Percent | Percent | Percent |
| Injury | injury | Injury | injury |
| 14 DAT | 21 DAT | 14 DAT | 21 DAT |

CYP14

| | | | | |
|---|---|---|---|---|
| Event 267 | 32 | 20 | 79 | 57 |
| Event 309 | 37 | 22 | 90 | 89 |
| Event 280 | 19 | 12 | 64 | 35 |
| Event 324 | 48 | 30 | 75 | 66 |

CYP15

| | | | | |
|---|---|---|---|---|
| Event 784 | 8 | 11 | 78 | 44 |
| Event 794 | 12 | 9 | 78 | 49 |
| Event 618 | 23 | 12 | 60 | 32 |
| Event 670 | 10 | 15 | 52 | 34 |

Control

| | | | |
|---|---|---|---|
| 61 | 49 | 90 | 91 |

At the lower 5 g ai/ha rate, many of the CYP14 and CYP15 transgenic events showed less than 30% injury and often less than 20% injury, whereas the treated control (wild-type) plants trended around 50% injury at the 5 g ai/ha rate. At the higher treatment rate (25 g ai/ha), there was a corresponding increase in plant injury and more variability, with some events trending around 30% injury, and other events with injury rates near that of the treated controls (around 90% injury). Transgenic plants expressing CYP12_var or CYP13 did not show any noticeable tolerance to icafolin (see Table 3), as injury rates from both the lower 5 g ai/ha rate and the higher 25 g ai/ha rate were similar to those observed in the treated control plants. When coupled with the data from 37 other CYPs tested in the transgenic plant screening assay, these results suggest that only CYP14 and CYP15 are capable of providing significant tolerance to icafolin herbicide.

### Example 2: Herbicide Tolerance Evaluation of CYP15-Expressing Transgenic Plants

This example describes the results of evaluating the tolerance of CYP15-expressing transgenic soybean plants to four different classes of herbicides.

Screening assays were designed to determine whether CYP15-expressing transgenic soybean plants demonstrated increased tolerance to herbicides other than icafolin. Transgenic soybean plants expressing CYP15 and control (wild-type) plants were treated with eight common herbicides, representing four different families of herbicides, as shown in Table 5.

TABLE 5
Herbicides Tested on CYP15 Transgenic Soybean Plants.

| Herbicide | Herbicide | |
|---|---|---|
| trade | common | |
| name | name | Herbicide family |
| AccentQ | Nicosulfuron | Sulfonylurea/Acetolactate synthase |
| | | (ALS) inhibitor |
| Revolver | Foramsulfuron | Sulfonylurea/Acetolactate synthase |
| | | (ALS) inhibitor |
| Laudis | Tembotrione | 4-hydroxyphenylpyruvate dioxygenase |
| | | (HPPD) inhibitor |
| Callisto | Mesotrione | 4-hydroxyphenylpyruvate dioxygenase |
| | | (HPPD) inhibitor |
| Sharpen | Saflufenacil | Protoporphyrinogen IX oxidase (PPO) |
| | | inhibitor |
| Spartan | Sulfentrazone | Protoporphyrinogen IX oxidase (PPO) |
| | | inhibitor |
| 2,4-D amine | 2,4-D | Synthetic auxin |
| XtendiMax | Dicamba | Synthetic auxin |

The CYP15-expressing plants were grown in the greenhouse, along with control plants, and challenged with the herbicides listed in Table 5 at the V2-V3 growth stage in a manner similar to that of the icafolin spray applications described in Example 1. To assay for complete and partial tolerance, herbicides were applied at full field (1×) rates and half field (0.5×) rates, respectively. The treated soybean plants were returned to the greenhouse following spray application and allowed to grow under standard conditions. The plants were rated for herbicide injury at approximately 14 DAT and 21 DAT following spray application. Untreated transgenic plants and control plants were used for phenotypic comparison. The herbicide injury rating accounts for plant malformation, stunting, chlorosis, necrosis, and plant death relative to the untreated control plants. An injury rating of approximately 90% or above indicates nearly complete plant death. An injury rating of less than 30% indicates partial tolerance and less than 20% indicates good tolerance. Injury ratings were collected and analyzed, and photographs of representative plants were taken. The injury ratings are summarized in Table 6.

TABLE 6
Herbicide Injury Ratings (in %) of CYP15
Transgenic Soybean Plants at 14 DAT.

| | |
|---|---|
| Control (Wild-type) | CYP15 |

| Herbicide | Percent | | Percent | |
|---|---|---|---|---|
| treatment | injury | St. Dev | injury | St. Dev |
| AccentQ (1/2x) | 62 | 4.1 | 53 | 15.0 |
| AccentQ (1x) | 64 | 4.9 | 60 | 7.1 |
| Revolver (1/2x) | 61 | 6.6 | 50 | 7.1 |
| Revolver 1x | 60 | 0.0 | 57 | 6.7 |
| Laudis 1/2x | 85 | 0.0 | 85 | 0.0 |
| Laudis 1x | 92 | 2.6 | 86 | 2.2 |
| Callisto 1/2x | 65 | 3.2 | 61 | 2.5 |
| Callisto 1x | 67 | 2.6 | 63 | 2.7 |
| Sharpen 1/2x | 68 | 19.4 | 63 | 15.7 |
| Sharpen 1x | 88 | 6.1 | 89 | 2.2 |
| Spartan 1/2x | 29 | 2.0 | 33 | 9.7 |
| Spartan 1x | 28 | 2.6 | 30 | 0.0 |
| 2,4-D amine 1/2x | 75 | 0.0 | 72 | 6.7 |
| 2,4-D amine 1x | 77 | 6.1 | 95 | 10.0 |
| XtendiMax 1/2x | 75 | 0.0 | 76 | 2.2 |
| XtendiMax 1x | 81 | 3.8 | 85 | 0.0 |

The results demonstrate that herbicide injury ranged from approximately 30% to over 90% at 14 DAT. No notable difference in the injury ratings were observed at the 14 DAT timepoint in the CYP15-expressing plants relative to the control plants. At the 21 DAT timepoint, some variability was observed in the regrowth of plants between treatments, however, both the control plants and the CYP15-expressing plants showed approximately 40% injury or higher across all treatments, suggesting the plants were not tolerant. Taken together, the data suggest that expression of CYP15 in soybean plants did not provide tolerance to nicosulfuron, foramsulfuron, tembotrione, mesotrione, saflufenacil, sulfentrazone, 2,4-D or dicamba.

### Example 3: Icafolin Herbicide Tolerance Evaluation of Additional CYP Proteins

This example describes testing the ability of additional cytochrome P450 genes to confer tolerance to icafolin herbicide to plants.

Public and internal plant genome databases, including Amaranthus databases, were screened to identify additional cytochrome P450 genes to test for icafolin tolerance. Twenty-one additional cytochrome P450 genes, including nine Amaranthus CYP genes were selected and screened for icafolin tolerance. Among these was CYP21 (SEQ ID NO:16) sourced from Amaranthus palmeri and CYP28 (SEQ ID NO:23) sourced from Amaranthus tuberculatus. At the amino acid level, CYP21 and CYP28 share 98.8% identity and 95.1% identity, respectively, with CYP15.

The transgenic plant-based screening approach described in Example 1 was employed to screen the selected CYPs for icafolin tolerance. For plant transformation, the protein sequences encoding the CYP enzymes were codon optimized for dicot expression. Table 7 provides the sequence identifiers (SEQ ID NOs) corresponding to the protein and nucleotide sequences.

TABLE 7
Amaranthus palmeri Cytochrome p450 genes.

| | | | Amaranthus | Dicot codon |
|---|---|---|---|---|
| Cytochrome | CYP | Protein | palmeri DNA | optimized DNA |
| P450 gene | Subfamily | Sequence | sequence | sequence |
| CYP21 | CYP81E | SEQ ID NO: 16 | SEQ ID NO: 18 | SEQ ID NO: 20 |
| CYP28 | CYP81E | SEQ ID NO: 23 | SEQ ID NO: 24 | SEQ ID NO: 25 |

The codon-optimized genes were placed into plant transformation cassettes with each CYP coding sequence operably linked to a constitutive expression element comprising the promoter, leader, and intron of Arabidopsis thaliana Ubiquitin 3 (SEQ ID NO:14) and a Medicago truncatula photosystem II terminator (SEQ ID NO:15). Two plant transformation vectors were created, each comprising one of the CYP gene cassettes and an aadA selectable marker expression cassette, conferring resistance to the antibiotics spectinomycin and streptomycin. Soybean cultivar AG3555 seed-derived embryo explants were transformed with the vectors described above by Agrobacterium-mediated transformation using known methods in the art, and positive-transformed plantlets were selected based on growth in the presence of spectinomycin. Plantlets comprising single copy events were grown in the greenhouse to produce R₁ seeds. R₁ plants were subsequently selected for the homozygous CYP positive plants, either by molecular analysis of young leaf tissue or by seed chipping prior to planting. These selected plants were then screened for tolerance to the icafolin herbicide.

Herbicide tolerance screening was performed by selecting R₁ plants at roughly the V2-V3 developmental stage and applying the icafolin herbicide in a track sprayer under standard greenhouse conditions. Two independent transgenic events per transgene were selected for screening. The herbicide was formulated with 1% v/v methylated seed oil (MSO) and 2.5% v/v ammonium sulfate (AMS) and sprayed with a XR nozzle at 15 gallons per hectare (GPA) to mimic field conditions. The icafolin formulation was sprayed at two rates: 5 g active ingredient (ai)/hectare (ha) and 25 g ai/ha. The treated soybean plants were returned to the greenhouse following spray application and allowed to grow under standard conditions. The plants were rated for herbicide injury at approximately 14 and 21 days after treatment (DAT). Untreated transgenic plants and control (wild-type) plants were used for phenotypic comparison. The herbicide injury rating accounts for plant malformation, stunting, chlorosis, necrosis, and plant death relative to the untreated control plants. An injury rating of approximately 90% or above indicates nearly complete plant death. An injury rating of less than 30% indicates partial tolerance and an injury rating of less than 20% indicates good tolerance. Injury ratings were collected and analyzed, and photographs of representative plants were taken. The injury ratings are summarized in Table 8.

TABLE 8
Injury ratings (in %) Following Application
of Icafolin Herbicide in Soybean.

| 5 g ai/ha icafolin treatment | | 25 g ai/ha icafolin treatment | |
|---|---|---|---|
| Percent | Percent | Percent | Percent |
| Injury | injury | Injury | injury |
| 14 DAT | 21 DAT | 14 DAT | 21 DAT |

CYP21

| | | | | |
|---|---|---|---|---|
| Event 344 | 2.5 | 5.0 | 50 | 37.5 |
| Event 502 | 7.5 | 5.0 | 72.5 | 62.5 |

CYP28

| | | | | |
|---|---|---|---|---|
| Event 44 | 15.6 | 13.3 | 67.2 | 56.7 |
| Event 90 | 28.0 | 13.0 | 68.0 | 74.0 |

Control

| | | | | |
|---|---|---|---|---|
| Control | 45.0 | 35.6 | 78.1 | 83.1 |

At 14 DAT, at the lower 5 g ai/ha rate, the CYP21 transgenic events showed less than 10% injury, and the CYP28 transgenic events showed less than 30% injury. The treated control (wild-type) plants trended around 45% injury at the 5 g ai/ha rate. At the higher treatment (25 g ai/ha) rate, there was a corresponding increase in plant injury and more variability. The control plants showed about 78% injury. One CYP21 event showed about 50% injury, while the other event had an injury rating closer to that of the treated control at about 72% injury. The CYP28 events showed injury ratings trending around 67.5%. The same effects were apparent in the 21 DAT ratings. Overall, the data demonstrate that CYP21 provides good tolerance to icafolin, and CYP28 provides partial tolerance to icafolin at the lower spray rates. None of the other nineteen CYP proteins that were tested showed icafolin tolerance.

### Example 4: Icafolin Herbicide Tolerance Evaluation of Additional CYP Proteins

This example describes testing the ability of the cytochrome P450 gene CYP34 to confer tolerance to icafolin herbicide to plants.

CYP34 (SEQ ID NO:26) was sourced from Amaranthus cruentus. At the amino acid level, CYP34 shares 96.1% identity with CYP15.

The transgenic plant-based screening approach described in Example 1 was used to screen CYP34 for icafolin tolerance. For plant transformation, the protein sequence encoding CYP34 enzyme was codon optimized for dicot expression. Table 9 provides the sequence identifiers (SEQ ID NOs) corresponding to the protein and nucleotide sequences.

TABLE 9
Amaranthus cruentus Cytochrome p450 gene.

| | | | Amaranthus | Dicot codon |
|---|---|---|---|---|
| | | | cruentus | optimized |
| Cytochrome | CYP | Protein | DNA | DNA |
| P450 gene | Subfamily | Sequence | sequence | sequence |
| CYP34 | CYP81E | SEQ ID NO: 26 | SEQ ID NO: 29 | SEQ ID NO: 32 |

The codon-optimized gene was placed into plant transformation cassette with the CYP34 coding sequence operably linked to a constitutive expression element comprising the promoter, leader, and intron of Arabidopsis thaliana Ubiquitin 3 (SEQ ID NO:14) and a Medicago truncatula photosystem II terminator (SEQ ID NO:15). A plant transformation vector was created, comprising the CYP34 gene cassette and an aadA selectable marker expression cassette, which confers resistance to the antibiotics spectinomycin and streptomycin. Soybean cultivar AG3555 seed-derived embryo explants were transformed with the vector described above by Agrobacterium-mediated transformation using known methods in the art, and positive-transformed plantlets were selected based on growth in the presence of spectinomycin. Plantlets comprising single copy events were grown in the greenhouse to produce R₁ seeds. R₁ plants were subsequently selected for the homozygous CYP positive plants, either by molecular analysis of young leaf tissue or by seed chipping prior to planting. These selected plants were then screened for tolerance to the icafolin herbicide.

Herbicide tolerance screening was performed by selecting R₁ plants at roughly the V2-V3 developmental stage and applying the icafolin herbicide in a track sprayer under standard greenhouse conditions. Two independent transgenic events per transgene were selected for screening. The herbicide was formulated with 1% v/v methylated seed oil (MSO) and 2.5% v/v ammonium sulfate (AMS) and sprayed with a XR nozzle at 15 gallons per hectare (GPA) to mimic field conditions. The icafolin formulation was sprayed at two rates: 5 g active ingredient (ai)/hectare (ha) and 25 g ai/ha. The treated soybean plants were returned to the greenhouse following spray application and allowed to grow under standard conditions. The plants were rated for herbicide injury at approximately 13 and 21 days after treatment (DAT). Untreated transgenic plants and control (wild-type) plants were used for phenotypic comparison. The herbicide injury rating accounts for plant malformation, stunting, chlorosis, necrosis, and plant death relative to the untreated control plants. An injury rating of approximately 90% or above indicates nearly complete plant death. An injury rating of less than 30% indicates partial tolerance and an injury rating of less than 20% indicates good tolerance. Injury ratings were collected and analyzed, and photographs of representative plants were taken. The injury ratings are summarized in Table 10.

TABLE 10
Injury ratings (in %) Following Application
of Icafolin Herbicide in Soybean.

| 5 g ai/ha icafolin treatment | | 25 g ai/ha icafolin treatment | |
|---|---|---|---|
| Percent | Percent | Percent | Percent |
| Injury | injury | Injury | injury |
| 13 DAT | 21 DAT | 13 DAT | 21 DAT |

CYP34

| | | | | |
|---|---|---|---|---|
| Event 326 | 12.5 | 9.2 | 48.3 | 36.7 |
| Event 356 | 13.3 | 12.5 | 35.8 | 30.8 |

Control

| | | | | |
|---|---|---|---|---|
| Control | 42.5 | 35.0 | 70.0 | 79.4 |

At 13 DAT, at the lower 5 g ai/ha rate, the CYP34 transgenic events showed less than 15% injury. The treated control (wild-type) plants trended around 42% injury at the 5 g ai/ha rate. At the higher treatment (25 g ai/ha) rate, the control plants showed about 70% injury while the CYP34 events showed less than 50% injury. A similar trend was observed in the 21 DAT ratings with the CYP34 transgenic events showing less injury as compared to the control plants. Overall, the data demonstrate that CYP34 provides good tolerance to icafolin.

### Example 5: Field Testing of Selected CYP14 and CYP15-Expressing Soybean Plants

Soy transgenic plants expressing CYP14 or CYP15 were assayed in small scale field trails across nine sites in the United States for tolerance to icafolin spray applications. The plants were grown in field plots and sprayed during the early vegetative stages when plants were roughly at the V3 stage. The icafolin formulation was sprayed at either a 5 g ai/ha or 25 g ai/ha application rate with 2 treatment replications per location. The plants were rated for herbicide injury at 7, 14, 21, and 28 days after treatment (DAT). A total of 12 events for CYP14 and 11 events for CYP15 were tested. An event transformed with a non-herbicide tolerance construct served as a negative control to provide a comparison for injury.

The injury data at 21 DAT are presented in Table 11. Similar to what was observed in the greenhouse testing, both CYP14 and CYP15 provided visual tolerance to the icafolin application at the 5 g ai/ha rate with injury ratings below 15% injury. Incomplete tolerance was observed at the higher 25 g ai/ha application rate, with plants showing less injury than the control but incurring significant injury typical of icafolin injury characterized by stunting, malformed leaves, and arrested development. This indicates that expression of both CYP14 and CYP15 using the expression cassette described in Example 1 provides tolerance in soy to low application rates of icafolin in both greenhouse and field growth conditions.

TABLE 11
Summary of Field Injury Ratings (in %) for Plants Expressing
CYP14 or CYP15 Following Application of Icafolin Herbicide
in Soybean. Injury Shown is at 21 DAT and Averaged Across
Repetitions, Field Locations, and Events.

Percent Plant Injury (21 DAT)

| Cytochrome | 5 g ai/ha icafolin | 25 g ai/ha icafolin |
|---|---|---|
| p450 gene | treatment | treatment |
| CYP14 | 11.4 | 41.1 |
| CYP15 | 8.7 | 31.9 |
| Control | 18.9 | 62.8 |

1. A recombinant DNA molecule comprising a heterologous promoter operably linked to a polynucleotide sequence that encodes a polypeptide comprising an amino acid sequence having at least about 94% sequence identity to SEQ ID NO:3, SEQ ID NO:4, SEQ ID NO:16, SEQ ID NO:17, SEQ ID NO:23, SEQ ID NO:26, SEQ ID NO:27, or SEQ ID NO:28, wherein the heterologous promoter is functional in a plant cell.

2. The recombinant DNA molecule of claim 1, wherein the polypeptide confers tolerance to an icafolin herbicide.

3. The recombinant DNA molecule of claim 11, wherein:
a) the polynucleotide sequence comprises a nucleic acid sequence having at least about 95%, at least about 96%, at least about 97%, at least about 98%, or at least about 99% sequence identity to SEQ ID NO:8, SEQ ID NO:9, SEQ ID NO:18, SEQ ID NO:19, SEQ ID NO:24, SEQ ID NO: 29, SEQ ID NO: 30, or SEQ ID NO:31;
b) the polynucleotide sequence comprises a nucleic acid sequence having at least about 85% sequence identity to SEQ ID NO:12, SEQ ID NO:13, SEQ ID NO:20, SEQ ID NO:21, SEQ ID NO:22, SEQ ID NO:25, SEQ ID NO: 32, SEQ ID NO:33, SEQ ID NO:34, SEQ ID NO: 35, or SEQ ID NO:36; or
c) the polypeptide comprises an amino acid sequence having at least 95%, at least 96%, at least 97%, at least 98%, or at least 99% identity to SEQ ID NO:3, SEQ ID NO:4, SEQ ID NO:16, SEQ ID NO:17, SEQ ID NO:23, SEQ ID NO:26, SEQ ID NO:27, or SEQ ID NO:28.

4-8. (canceled)

9. The recombinant DNA molecule of claim 3, wherein;
a) the polypeptide comprises the amino acid sequence of SEQ ID NO:3, SEQ ID NO:4, SEQ ID NO:16, SEQ ID NO:17, SEQ ID NO:23, SEQ ID NO:26, SEQ ID NO:27, or SEQ ID NO:28; or
b) the nucleic acid sequence is selected from the group consisting of SEQ ID NO:8, SEQ ID NO:9, SEQ ID NO:12, SEQ ID NO:13, SEQ ID NO:18, SEQ ID NO:19, SEQ ID NO:20, SEQ ID NO:21, SEQ ID NO:22, SEQ ID NO:24, SEQ ID NO:25, SEQ ID NO:29, SEQ ID NO:30, SEQ ID NO:31, SEQ ID NO:32, SEQ ID NO:33, SEQ ID NO:34, SEQ ID NO:35 and SEQ ID NO:36.

10-14. (canceled)

15. The recombinant DNA molecule of claim 11, wherein:
a) the polynucleotide sequence further comprises a nucleic acid sequence that encodes a targeting sequence that functions to localize the encoded polypeptide within a cell; or
b) the recombinant DNA molecule is integrated into the genome of a transgenic plant, seed, plant part, or cell.

16. The recombinant DNA molecule of claim 2, wherein said icafolin herbicide is selected from the group consisting of:
methyl(2R*,4R*)-4-[[(5S)-3-(3,5-difluorophenyl)-5-vinyl-4H-isoxazole-5-carbonyl]amino]tetrahydrofuran-2-carboxylate;
methyl(2R,4R)-4-[[(5S)-3-(3,5-difluorophenyl)-5-vinyl-4H-isoxazole-5-carbonyl]-amino]tetrahydrofuran-2-carboxylate;
methyl (2S,4S)-4-[[(5S)-3-(3,5-difluorophenyl)-5-vinyl-4H-isoxazole-5-carbonyl]amino]tetrahydrofuran-2-carboxylate;
(2R*,4R*)-4-[[(5S)-3-(3,5-difluorophenyl)-5-vinyl-4H-isoxazole-5-carbonyl]amino]tetrahydrofuran-2-carboxylic acid;
(2R,4R)-4-[[(5S)-3-(3,5-difluorophenyl)-5-vinyl-4H-isoxazole-5-carbonyl]amino]tetrahydrofuran-2-carboxylic acid;
(2S,4S)-4-[[(5S)-3-(3,5-difluorophenyl)-5-vinyl-4H-isoxazole-5-carbonyl]amino]tetrahydrofuran-2-carboxylic acid; and
combinations of any thereof.

17. A DNA construct comprising the recombinant DNA molecule of claim 1.

18. (canceled)

19. A transgenic plant, seed, cell, or plant part comprising a recombinant DNA molecule comprising a heterologous promoter operably linked to a polynucleotide sequence that encodes a polypeptide comprising an amino acid sequence having at least about 85% sequence identity to SEQ ID NO:3, SEQ ID NO:4, SEQ ID NO:16, SEQ ID NO: 17, SEQ ID NO:23, SEQ ID NO: 26, SEQ ID NO:27, or SEQ ID NO:28.

20. The transgenic plant, seed, cell, or plant part of claim 19, wherein;
a) the transgenic plant, seed, cell, or plant part comprises tolerance to an icafolin herbicide; or
b) the transgenic plant, seed, cell, or plant part is a soybean, a maize, a cotton, a barley, a sorghum, a rice, a wheat, a sugar cane, a sugar beet, an alfalfa, or a Brassica plant, seed, cell, or plant part.

21. (canceled)

22. The transgenic plant, seed, cell, or plant part of claim 20, wherein:
a) the icafolin herbicide is selected from the group consisting of:
methyl(2R*,4R*)-4-[[(5S)-3-(3,5-difluorophenyl)-5-vinyl-4H-isoxazole-5-carbonyl]amino]tetrahydrofuran-2-carboxylate;
methyl(2R,4R)-4-[[(5S)-3-(3,5-difluorophenyl)-5-vinyl-4H-isoxazole-5-carbonyl]-amino]tetrahydrofuran-2-carboxylate;
methyl (2S,4S)-4-[[(5S)-3-(3,5-difluorophenyl)-5-vinyl-4H-isoxazole-5-carbonyl]amino]tetrahydrofuran-2-carboxylate;
(2R*,4R*)-4-[[(5S)-3-(3,5-difluorophenyl)-5-vinyl-4H-isoxazole-5-carbonyl]amino]tetrahydrofuran-2-carboxylic acid;
(2R,4R)-4-[[(5S)-3-(3,5-difluorophenyl)-5-vinyl-4H-isoxazole-5-carbonyl]amino]tetrahydrofuran-2-carboxylic acid;
(2S,4S)-4-[[(5S)-3-(3,5-difluorophenyl)-5-vinyl-4H-isoxazole-5-carbonyl]amino]tetrahydrofuran-2-carboxylic acid; and
combinations of any thereof; or
b) the transgenic plant, seed, cell, or plant part comprises tolerance to at least one additional herbicide.

23. (canceled)

24. A method of conferring herbicide tolerance to a plant, seed, cell, or plant part, the method comprising expressing the recombinant DNA molecule of claim 1 in said plant, seed, cell, or plant part.

25. The method of claim 24, wherein said plant, seed, cell, or plant part comprises tolerance to an icafolin herbicide.

26. The method of claim 25, wherein the icafolin herbicide is selected from the group consisting of:
methyl(2R*,4R*)-4-[[(5S)-3-(3,5-difluorophenyl)-5-vinyl-4H-isoxazole-5-carbonyl]amino]tetrahydrofuran-2-carboxylate;
methyl(2R,4R)-4-[[(5S)-3-(3,5-difluorophenyl)-5-vinyl-4H-isoxazole-5-carbonyl]-amino]tetrahydrofuran-2-carboxylate;
methyl (2S,4S)-4-[[(5S)-3-(3,5-difluorophenyl)-5-vinyl-4H-isoxazole-5-carbonyl]amino]tetrahydrofuran-2-carboxylate;
(2R*,4R*)-4-[[(5S)-3-(3,5-difluorophenyl)-5-vinyl-4H-isoxazole-5-carbonyl]amino]tetrahydrofuran-2-carboxylic acid;
(2R,4R)-4-[[(5S)-3-(3,5-difluorophenyl)-5-vinyl-4H-isoxazole-5-carbonyl]amino]tetrahydrofuran-2-carboxylic acid;
(2S,4S)-4-[[(5S)-3-(3,5-difluorophenyl)-5-vinyl-4H-isoxazole-5-carbonyl]amino]tetrahydrofuran-2-carboxylic acid; and
combinations of any thereof.

27. A method of producing a transgenic plant or part thereof, the method comprising:
a) introducing a recombinant DNA molecule comprising a heterologous promoter operably linked to a polynucleotide sequence that encodes a polypeptide comprising an amino acid sequence having at least about 85% sequence identity to SEQ ID NO:3, SEQ ID NO:4, SEQ ID NO:16, SEQ ID NO:17, SEQ ID NO:23, SEQ ID NO:26, SEQ ID NO:27, or SEQ ID NO:28 into a plant cell; and
b) regenerating the transgenic plant or part thereof from said cell or a descendant cell thereof that comprises the recombinant DNA molecule.

28. The method of claim 27, further comprising selecting a regenerated transgenic plant comprising tolerance to an icafolin herbicide.

29. The method of claim 28, wherein the icafolin herbicide is selected from the group consisting of:
methyl(2R*,4R*)-4-[[(5S)-3-(3,5-difluorophenyl)-5-vinyl-4H-isoxazole-5-carbonyl]amino]tetrahydrofuran-2-carboxylate;
methyl(2R,4R)-4-[[(5S)-3-(3,5-difluorophenyl)-5-vinyl-4H-isoxazole-5-carbonyl]-amino]tetrahydrofuran-2-carboxylate;
methyl (2S,4S)-4-[[(5S)-3-(3,5-difluorophenyl)-5-vinyl-4H-isoxazole-5-carbonyl]amino]tetrahydrofuran-2-carboxylate;
(2R*,4R*)-4-[[(5S)-3-(3,5-difluorophenyl)-5-vinyl-4H-isoxazole-5-carbonyl]amino]tetrahydrofuran-2-carboxylic acid;
(2R,4R)-4-[[(5S)-3-(3,5-difluorophenyl)-5-vinyl-4H-isoxazole-5-carbonyl]amino]tetrahydrofuran-2-carboxylic acid;
(2S,4S)-4-[[(5S)-3-(3,5-difluorophenyl)-5-vinyl-4H-isoxazole-5-carbonyl]amino]tetrahydrofuran-2-carboxylic acid; and
combinations of any thereof.

30. The method of claim 27, further comprising:
a) crossing the regenerated transgenic plant with itself or with a second plant to produce seed; or
b) crossing the regenerated transgenic plant, or a descendant plant thereof that comprises the recombinant DNA molecule, with itself or a second plant to produce seed; wherein the seed comprises the recombinant DNA molecule.

31. The method of claim 27, wherein;
a) the polypeptide comprises the amino acid sequence of SEQ ID NO:3, SEQ ID NO:4, SEQ ID NO:16, SEQ ID NO:17, SEQ ID NO:23, SEQ ID NO:26, SEQ ID NO:27, or SEQ ID NO:28; or
b) the polynucleotide sequence comprises a nucleic acid sequence having at least about 95% sequence identity to SEQ ID NO:8, SEQ ID NO:9, SEQ ID NO:18, SEQ ID NO:19, SEQ ID NO:24, SEQ ID NO:29, SEQ ID NO:30, or SEQ ID NO:31, or at least about 85% sequence identity to SEQ ID NO:12, SEQ ID NO:13, SEQ ID NO:20; SEQ ID NO:21, SEQ ID NO:22, SEQ ID NO:25, SEQ ID NO:32, SEQ ID NO:33, SEQ ID NO:34, SEQ ID NO:35, or SEQ ID NO:36.

32-33. (canceled)

34. A transgenic plant or part thereof produced by the method of claim 27, wherein said transgenic plant or part thereof comprises the recombinant DNA molecule.

35. A seed produced by the method of claim 30.

36. A method for controlling weeds in a plant growth area that comprises the transgenic plant or seed of claim 19, the method comprising contacting the plant growth area with an icafolin herbicide, wherein the transgenic plant or seed is tolerant to the icafolin herbicide, and wherein weeds are controlled in the plant growth area.

37. The method of claim 36, wherein;
a) the icafolin herbicide is selected from the group consisting of:
methyl(2R*,4R*)-4-[[(5S)-3-(3,5-difluorophenyl)-5-vinyl-4H-isoxazole-5-carbonyl]amino]tetrahydrofuran-2-carboxylate;
methyl(2R,4R)-4-[[(5S)-3-(3,5-difluorophenyl)-5-vinyl-4H-isoxazole-5-carbonyl]-amino]tetrahydrofuran-2-carboxylate;
methyl (2S,4S)-4-[[(5S)-3-(3,5-difluorophenyl)-5-vinyl-4H-isoxazole-5-carbonyl]amino]tetrahydrofuran-2-carboxylate;
(2R*,4R*)-4-[[(5S)-3-(3,5-difluorophenyl)-5-vinyl-4H-isoxazole-5-carbonyl]amino]tetrahydrofuran-2-carboxylic acid;
(2R,4R)-4-[[(5S)-3-(3,5-difluorophenyl)-5-vinyl-4H-isoxazole-5-carbonyl]amino]tetrahydrofuran-2-carboxylic acid;
(2S,4S)-4-[[(5S)-3-(3,5-difluorophenyl)-5-vinyl-4H-isoxazole-5-carbonyl]amino]tetrahydrofuran-2-carboxylic acid; and
combinations of any thereof; or
b) the transgenic plant or seed is a soybean, a maize, a cotton, a barley, a sorghum, a rice, a wheat, a sugar cane, a sugar beet, an alfalfa, or a Brassica plant or seed.

38. (canceled)

39. A method of identifying a plant having tolerance to an icafolin herbicide and to at least one additional herbicide, the method comprising:
a) obtaining the transgenic plant according to claim 20;
b) applying the at least one additional herbicide to said plant or a part thereof; and
c) identifying said plant as exhibiting tolerance to the at least one additional herbicide.

40. A method for reducing the development of herbicide tolerant weeds in a plant growth area comprising the transgenic plant or seed according to claim 20, the method comprising contacting the plant growth area with an icafolin herbicide and at least one additional herbicide, wherein the transgenic plant or seed is tolerant to the icafolin herbicide and the at least one additional herbicide.

41. The method of claim 40, wherein;
a) the icafolin herbicide is selected from the group consisting of:
methyl(2R*,4R*)-4-[[(5S)-3-(3,5-difluorophenyl)-5-vinyl-4H-isoxazole-5-carbonyl]amino]tetrahydrofuran-2-carboxylate;
methyl(2R,4R)-4-[[(5S)-3-(3,5-difluorophenyl)-5-vinyl-4H-isoxazole-5-carbonyl]-amino]tetrahydrofuran-2-carboxylate;
methyl (2S,4S)-4-[[(5S)-3-(3,5-difluorophenyl)-5-vinyl-4H-isoxazole-5-carbonyl]amino]tetrahydrofuran-2-carboxylate;
(2R*,4R*)-4-[[(5S)-3-(3,5-difluorophenyl)-5-vinyl-4H-isoxazole-5-carbonyl]amino]tetrahydrofuran-2-carboxylic acid;
(2R,4R)-4-[[(5S)-3-(3,5-difluorophenyl)-5-vinyl-4H-isoxazole-5-carbonyl]amino]tetrahydrofuran-2-carboxylic acid;
(2S,4S)-4-[[(5S)-3-(3,5-difluorophenyl)-5-vinyl-4H-isoxazole-5-carbonyl]amino]tetrahydrofuran-2-carboxylic acid; and
combinations of any thereof; or
b) the at least one additional herbicide is selected from the group consisting of: an ACCase inhibitor, an ALS inhibitor, an EPSPS inhibitor, a synthetic auxin, a photosynthesis inhibitor, a glutamine synthesis inhibitor, a HPPD inhibitor, a PPO inhibitor, a PDS inhibitor, and a long-chain fatty acid inhibitor.

42. (canceled)


SEQUENCE LISTING (36 sequences)

SEQ ID NO: 1 - protein, 517 aa, Amaranthus palmeri
MEETSILSIGISLVCVVLVSVSWGILNWVWFKPKRLEKCLRNQGFNGNSYRLLYGDTNERARITMESRLNPMPNLSNHFLPHSLAFFHHIITKYGRRCFIWNGPIPLVNIEEPYLIREIFMKINDFQKVKSNPIIKKVAPGLLFLEGHAWAQRRKLLTPAFHIDKLKFMVPTIWESSKEMIKEWEELASKTGSCEIEVWSSLQKLSADMISRAAFGSSYEEGKTIFDLLTEQIKIVVPVFNSVYIPGWRFLPTKTNRRISEIDREIRIRLKGIIDKRKNTINREERAKDDLLSTLIQDNFGHANKKQDINKLSIEEVVNECKLFYLAGQDTTSSLLLWTLIMLGKHQIWQQRAREEIINAFGDAKPQFHELNSLKIVNMILQEVLRLYPPVLELNREVNQDIKIGDVILPAGVLLNIPILLLQQDEKIWGKDAKEFNPERFSEGISKASKGNMSFFAFGWGPRICLGSNFAMIEAKLTLVFILQRFVFELSSSYRHAPLNVGTLRPQFGAPIIFRRR

SEQ ID NO: 2 - protein, 522 aa, Amaranthus palmeri
MEEKSILSIGISLFCVILFSVSWKIINWVWLKPKKFEKLLKNQGFDGNSYRLLHGDTKDKAKMTMEVTSKPMPNLSNDFLPHTLAFFHHTISKFGRKCMIWNGPVPFASIAEPDLVREVFMKINQFQKPKLNPTANKVISGLITLEGNAWTHRRKLLTPAFHFDKLKFMVPALWESTKEMIEEWEELVSKTGSCEIEVWSSLQKLSADMISRAAFGSSYEEGKTIFDLITEQIKIAIPMFNSVYIPGSRFLPTKRNRRITQIDREIKNLLMGIIDKRKKAIKTQEKTKDDLLNTLLESSYEQEENGHGNKKNDINLNIEEILGECKLFYLAGQDTTASLLVWTLIMLAKHQNWQQLARQEIFNAFGDKIPQFHDLNHLKIVNMILQEVLRLYPPVTETNREVIQDIKLGNMILPAGVVVNIPILLMHHDEKIWGNDAKEFNPERFSEGISKASKGNMSLFAFGWGPRICIGSNFAMIEAKLTLVCILQRFVFELSPSYLHAPSAIGSLRPQFGAPIIFRPRI

SEQ ID NO: 3 - protein, 489 aa, Amaranthus palmeri
MEILFTYLSIFATFFFLYKIIQSLKPNNKKHPPSPPSYPLLGHLHLLKPPFHRTLQSLAQRYGPIFTLKLGLQRAVVVSSAWAAEECFGQNDVVFANRPKFIIGQHLGYNHSILIWSPYGDYWRNLRRVTTITMLSMRRINEAGPTRKLEIRNMIMNLLESGSGTQKVNMNEVLSKLARNFVMRIVNGKSWEKMIIKPPENLMTICDFLPFLRWVDFKGIEKDMKEKQVERDEFLQSLVDEFRESKKKGEDLGMNNTLIEQLLDLEQAEPDQYSDETIKGIILVMLLAGSETTARTLEWALSNLINHPEILAKARTEIDLHVGKERLVDDSDLPKLTYTRCIVYETLRLFPAAPLLVPHFSSQDCTIGGYHVPKGTMLFVNAWAIHRDPTLWDEPTVFKPERFEKEKEGFKFMPFGIGRRSCPGNNVALRNVSLTLATLIQCFDWEAAESGSIDLTEKRGAGAIIVPKEKPLEAICRPRPSMEDFLAKI

SEQ ID NO: 4 - protein, 489 aa, Amaranthus palmeri
MEILFTYLSIFATFFFLYKIIQSLKTNNKKHPPSPPSYPLLGHLHLLKPPFHRTLQSLAQRYGPIFTLKLGLQRAVVVSSAWAAEECFGQNDVVFANRPKFIIGQHLGYNHSILIWSPYGDYWRNLRRVTTITMLSMRRINEAGPTRKLEIRNMIMNLLESGSGTQKVNMNEVLSKLARNFVMRIVNGKSWEKMIIKPPENLMTICDFLPFLRWVDFKGIEKDMKEKQVERDEFLQSLVDEFRESKKKGEDLGINNTLIEQLLDLQQAEPDQYSDETIKGIILVMLLAGSETTARTLEWALSNLINHPEILVKARTEIDLHVGKERLVDDSDLPKLTYTRCIVYETLRLFPAAPLLVPHFSSQDCTIGGYHVPKGTMLFVNAWAIHRDPTLWDEPTVFKPERFEKEKEGFKFMPFGIGRRSCPGNNVALRNVSLTLATLIQCFDWEAAESGSIDLTEKRGVGAIIVPKEKPLEAICRPRPSMEDFLAKI

SEQ ID NO: 5 - protein, 518 aa, synthetic construct
MSEETSILSIGISLVCVVLVSVSWGILNWVWFKPKRLEKCLRNQGFNGNSYRLLYGDTNERARITMESRLNPMPNLSNHFLPHSLAFFHHIITKYGRRCFIWNGPIPLVNIEEPYLIREIFMKINDFQKVKSNPIIKKVAPGLLFLEGHAWAQRRKLLTPAFHIDKLKFMVPTIWESSKEMIKEWEELASKTGSCEIEVWSSLQKLSADMISRAAFGSSYEEGKTIFDLLTEQIKIVVPVFNSVYIPGWRFLPTKTNRRISEIDREIRIRLKGIIDKRKNTINREERAKDDLLSTLIQDNFGHANKKQDINKLSIEEVVNECKLFYLAGQDTTSSLLLWTLIMLGKHQIWQQRAREEIINAFGDAKPQFHELNSLKIVNMILQEVLRLYPPVLELNREVNQDIKIGDVILPAGVLLNIPILLLQQDEKIWGKDAKEFNPERFSEGISKASKGNMSFFAFGWGPRICLGSNFAMIEAKLTLVFILQRFVFELSSSYRHAPLNVGTLRPQFGAPIIFRRR

SEQ ID NO: 6 - DNA, 1554 bp, Amaranthus palmeri
  source 1..1554: mol_type = other DNA
  CDS 1..1554: note = Amaranthus palmeri CDS encoding SEQ ID NO:1.
atggaagagacaagcatattatcaattggaatatcattggtttgtgttgtattagtaagtgtctcatggggaattctaaattgggtatggtttaagccaaaaaggttagaaaaatgccttagaaatcaagggtttaatggaaattcttataggttattgtatggagataccaatgaaagagctaggatcaccatggaatctagattaaacccaatgcccaacctatccaatcattttcttccacattctcttgcatttttccatcacattataactaaatatgggagaagatgctttatatggaatggacctattccattggtaaacatagaagaaccatatttaataagagaaatatttatgaaaataaatgattttcaaaaagtaaagtcaaatccaatcataaagaaggttgcaccgggcttgcttttcttagagggccatgcatgggctcaacgaaggaaactcctcactcctgctttccacatcgacaagctcaagtttatggttccaacaatatgggaaagttcaaaagaaatgattaaagaatgggaagaattggcatcaaaaacaggttcatgtgaaatagaagtttggtcatctttacaaaaattatcagcagatatgatttctagagctgcttttggaagtagttatgaagaagggaagacaatttttgatcttttaactgaacaaattaagattgtagttccagtatttaattctgtttatatccctggttggaggtttttaccaaccaaaacaaatcgaagaatatcagaaatcgacagagaaatcaggattcgactaaaaggaataattgacaaaagaaagaatacaataaatagagaagagagggcaaaggatgacttgcttagtacattaattcaagacaattttggacatgcaaataaaaagcaagacataaataagttaagtattgaagaagttgtaaatgaatgtaaattattttacttagctggacaagatacaacttcttcattattgttatggacactaattatgttgggaaaacaccaaatatggcaacaaagagcaagagaagagatcatcaatgcatttggggatgccaaacctcaatttcatgagttgaatagtcttaaaatagtaaacatgatactacaagaggttctaagattatatccaccagtattagaattaaaccgtgaagttaatcaagacataaaaataggagatgtaatacttccggcaggagttttacttaatataccaattttacttttgcaacaagatgaaaaaatatggggtaaagatgcgaaagaatttaatcccgaaagatttagtgaaggaatttcaaaagctagtaaaggaaatatgtcattctttgcgttcggttggggacctagaatatgtcttggatcgaattttgctatgattgaggccaaattaactttagtcttcattctgcaaagatttgtatttgagctttcatcatcttatcgtcatgcccctttgaatgttggaactcttagacctcaatttggtgcaccaattatatttcgtcgacgataa

SEQ ID NO: 7 - DNA, 1569 bp, Amaranthus palmeri
  source 1..1569: mol_type = other DNA
  CDS 1..1569: note = Amaranthus palmeri CDS encoding SEQ ID NO:2.
atggaagagaaaagcatattatcaattggaatatctttgttttgtgtaatattatttagtgtgtcatggaaaattataaattgggtatggttaaaaccaaaaaagtttgaaaaattgcttaaaaatcaagggtttgatggcaattcttataggttattgcatggagacaccaaagataaagctaagatgaccatggaagttacttctaagcctatgcctaatctatccaatgattttcttccacatactcttgcattttttcatcacactatatctaagttcggaagaaaatgcatgatatggaatgggccggttccatttgcaagcatagctgaaccggatttggtccgggaagtttttatgaagattaaccaatttcaaaagcctaagttaaaccctaccgcaaacaaggttatatccggtttgattaccttggaggggaatgcatggacccaccgaaggaaactccttacccctgctttccacttcgataaactcaagtttatggttccagcattatgggaaagtacaaaagaaatgattgaagaatgggaagaattggtatcaaaaacaggttcatgtgaaatagaagtttggtcatctttacaaaaattatcagcagatatgatttctagagctgcttttggaagtagttatgaagaagggaagacaatttttgatcttataactgaacaaattaaaattgcaattccaatgtttaattctgtttatatccccggttcgaggtttttgccaaccaagagaaatagaaggataacacaaattgatagagaaatcaagaatctactaatgggtattattgacaaaagaaagaaggcaataaaaactcaagaaaagacaaaggatgatttgcttaatacattattagagtcaagttatgagcaagaagaaaatgggcatggaaataaaaaaaatgatattaatttaaatattgaagaaattcttggtgaatgtaaattattttacctagcaggacaagatacaactgcttctttattggtatggacattaattatgttggcaaagcatcaaaattggcaacaactagcaagacaagagattttcaatgcatttggagataaaatacctcaatttcatgacttgaaccatcttaaaatagtaaatatgatactacaagaggtgctaagattatatccaccagtaacagaaacaaatcgtgaagtaatccaagacataaaattaggaaatatgatacttccagcaggtgtcgtagtgaatattccaattctacttatgcaccatgatgaaaagatatggggtaatgatgcgaaagaatttaatccagaaagatttagtgaaggaatatcaaaggctagtaaaggaaatatgtcattatttgcgtttggttggggacctaggatatgtattggatctaactttgctatgattgaggccaaactgacattggtttgcattttacaaaggtttgtatttgagctttcaccatcttatcttcacgccccttctgctattggaagtcttagaccccaatttggtgctccaatcatatttcgtccacgaatctag

SEQ ID NO: 8 - DNA, 1470 bp, Amaranthus palmeri
  source 1..1470: mol_type = other DNA
  CDS 1..1470: note = Amaranthus palmeri CDS encoding SEQ ID NO:3.
atggaaatattattcacttatttatccatctttgctacttttttcttcctctataaaatcatccaatccctcaaaccaaacaacaaaaaacacccaccaagcccaccatcatacccattattgggccacttacaccttctaaaacccccattccaccgtacactccaatctctagcccaacgttacggcccaatcttcactctcaaattgggccttcaacgggccgtggtagtttcctccgcatgggccgccgaagaatgtttcggtcaaaacgacgtcgtttttgcaaacagaccaaaattcataataggacaacatttaggatacaaccactctatccttatctggtccccatatggggactactggcggaacctccgacgtgtcacaaccattaccatgttatccatgagacggatcaacgaagcgggtccgacccggaaacttgagatccgaaacatgataatgaaccttctagaaagtgggagtgggacccaaaaggtgaatatgaatgaagtgctgagtaaactagcaagaaattttgtaatgaggatagtaaatggaaaatcatgggaaaaaatgataataaaaccccctgaaaatttgatgactatttgtgattttttaccatttttaagatgggtagattttaaagggattgaaaaagatatgaaggaaaaacaggttgaaagagatgagtttttacagagtttggttgatgaatttagagaaagtaagaaaaaaggtgaggatttagggatgaataatactttgattgaacaattgctggatttggaacaagctgaacctgatcaatactctgatgaaactatcaaaggaatcattttggtaatgttactagcgggatcagaaaccacagcacgaaccttagaatgggcattatcaaatctcattaaccacccagaaatcctcgcgaaagcaagaacagaaatcgacctccatgtaggcaaggaacgactagtagatgattctgatctaccaaaattaacgtatacacgttgcattgtatacgaaactctaagactatttccggctgcaccacttttggtacctcatttttcttcgcaagattgcaccataggagggtatcacgtaccaaaagggacaatgcttttcgtaaatgcttgggctatacacagagacccgaccctatgggacgagcctactgtgtttaagcccgagaggttcgaaaaggagaaagaagggtttaagtttatgccctttggaattggaaggagatcttgtccggggaacaatgtggctcttaggaatgtttcattgacattggctactcttattcagtgctttgattgggaagcagccgagagtggatcaattgatctaaccgaaaagcgtggtgctggtgcgatcattgtccctaaggaaaagccattggaggctatatgtcgaccacgaccctcaatggaggatttcctcgctaaaatttag

SEQ ID NO: 9 - DNA, 1470 bp, Amaranthus palmeri
  source 1..1470: mol_type = other DNA
  CDS 1..1470: note = Amaranthus palmeri CDS encoding SEQ ID NO:4.
atggaaatattattcacttatttatccatctttgctacttttttcttcctctataaaatcatccaatcccttaaaacaaacaataaaaaacacccaccaagcccaccatcatacccattattgggccacttacaccttctaaaacccccattccaccgtacactccaatctctagcccaacgttacggcccaatcttcactctcaaattgggccttcaacgggccgtggtagtttcctccgcatgggccgccgaagaatgtttcggtcaaaacgacgtcgtttttgcaaacagaccaaaattcataataggacaacatttaggatacaaccactctatccttatctggtccccatatggggactactggcggaacctccgacgtgtcacaaccattaccatgttatccatgagacggatcaacgaagcgggtccgacccggaaacttgagatccgaaacatgataatgaaccttctagaaagtggaagtgggacccaaaaggtgaatatgaatgaagtgctgagtaaactagcaagaaattttgtaatgaggatagtaaatggaaaatcatgggaaaaaatgataataaaaccccctgaaaatttaatgactatttgtgattttttaccatttttaagatgggtagattttaaagggatagaaaaggatatgaaggaaaaacaggttgaaagagatgagtttttacaaagtttggttgatgaatttagagaaagtaagaagaaaggtgaggatttagggataaataatactttgattgaacaattgctggatttgcaacaagctgaacctgatcaatactctgatgaaactatcaaaggaatcattttggtaatgttactagcaggatcagaaaccacagcacgaaccttagaatgggcattatcaaatctcataaaccacccagaaatcctcgtgaaagcaagaacagaaatcgacctccatgtaggcaaggaacgactagtagatgattctgatctaccaaaattaacgtatacacgttgcattgtatacgaaactctaagactatttccggctgcaccacttttagtacctcatttttcttcgcaagattgcaccataggaggctatcacgtaccaaaagggaccatgcttttcgtaaatgcttgggctatacacagagacccgaccctatgggacgagcctactgtgtttaagcctgagaggttcgagaaggagaaagaagggtttaagtttatgccctttggaattggaaggagatcttgtccagggaacaatgtggctcttaggaacgtttcattgacattggctactcttattcagtgctttgattgggaagcagctgagagtggatcaattgatctaaccgaaaagcgtggtgttggtgcgatcattgtccctaaggaaaagccattggaggctatatgtcgaccacgaccctcaatggaggatttcctcgctaaaatttag

SEQ ID NO: 10 - DNA, 1557 bp, synthetic construct
  source 1..1557: mol_type = other DNA
  misc_feature 1..1557: note = Artificial sequence.
atgagcgaggagacgtcgatcctgagcatcggcatatcgttggtctgcgtggtcctcgtttccgtatcgtggggaatcctgaactgggtctggttcaagcctaagcgtttggagaagtgcctacgcaatcaggggttcaacggcaactcataccggctgctctacggcgacaccaatgagcgcgcgcggatcactatggagagccgtctcaacccgatgccgaacctctccaaccacttcctaccacactcattggcattcttccatcacataatcactaagtacgggagaaggtgctttatctggaatgggcctataccgctcgtcaacattgaggagccatacctcataagggagatattcatgaagattaacgacttccagaaggtgaaaagcaaccccatcatcaagaaggtggcacccggacttctattcttggagggtcacgcctgggcgcagcgccgaaagctcttgacaccggccttccacattgacaagctcaaattcatggtgccgacgatatgggaaagcagtaaggagatgattaaggagtgggaggaactcgcctcaaagacgggctcctgcgagatcgaagtgtggtctagtttgcaaaagctctctgccgacatgatctcccgagccgccttcgggtcatcttacgaggagggcaagacaatcttcgacctactcacggagcagattaagatcgtcgtgccggtgttcaatagcgtgtacatcccagggtggcgattccttccgaccaagaccaaccgtcggatctcggagatcgaccgtgagataaggatacgtctgaaagggatcattgacaaacgcaagaacactatcaaccgtgaggagcgcgccaaagacgacctgctgagtaccctcatacaagacaacttcggacacgcaaacaagaagcaagacatcaacaagctgtccatcgaggaggtggtcaacgagtgcaagctgttctaccttgccgggcaagacacgacttcgagtttgctactctggaccctaatcatgctgggaaaacaccaaatctggcaacaacgcgcgagggaggaaatcatcaacgccttcggagacgcaaagcctcagttccatgagcttaactctctcaagatagtgaacatgatccttcaagaggtgctgcgcctctaccctcccgtgttggagctaaaccgcgaggtgaatcaggacatcaagatcggggacgtgatcctcccggccggagtcctgctgaacatccccattctgctccttcaacaagatgagaaaatctggggaaaggacgcgaaagagtttaaccccgagagattctcggaggggatctctaaggcgtcaaagggcaatatgtcattctttgcattcggctggggtccgcgaatctgtctcggctccaatttcgccatgatcgaggcgaagttgacactggtgttcatcctacaacggtttgtcttcgagctgtcttcaagttaccgtcacgctccgctgaacgtcggaaccctgcggccccagttcggtgcacctatcatcttccgacgacgctag

SEQ ID NO: 11 - DNA, 1569 bp, synthetic construct
  source 1..1569: mol_type = other DNA
  misc_feature 1..1569: note = Artificial sequence
atggaggagaaatccatcctatctatcggcatctccttgttctgcgtcatcttattctccgttagctggaagattatcaactgggtgtggctcaaacccaagaagttcgaaaagctactcaagaaccagggcttcgatgggaactcctaccgcctgctccacggcgacactaaggacaaggcgaagatgactatggaggtgacgtcgaaacccatgcccaacctttcgaacgacttcctcccgcacaccctcgcgttcttccaccacacaatctccaagttcgggcgcaagtgcatgatctggaacggacccgttcctttcgcaagtatcgcggagcctgacttggtgcgagaagtgttcatgaagatcaaccagtttcaaaagcctaagttaaacccgacggcaaacaaggtcatctcggggctcatcactttggaggggaacgcatggacccaccggaggaagctattgacaccggcttttcacttcgacaagctcaagttcatggtgcctgccctttgggagtccactaaggaaatgattgaggagtgggaggagttagttagcaagactggaagctgcgagattgaggtttggagcagcctacaaaagttgtcagcggacatgattagccgggcggcgttcgggtcatcgtacgaggagggcaaaaccatcttcgacctcatcaccgagcagatcaagatcgccattccaatgttcaactcggtgtacatccccggctctcgcttcctgcccaccaagaggaaccgacgaatcacgcagatagaccgggagatcaagaacctgctcatggggataattgacaaacgcaagaaggctatcaagacccaggagaaaactaaggacgacctcctgaacacgctgcttgaaagctcctacgagcaagaggagaacggccacgggaacaagaagaacgacattaaccttaacattgaggagattcttggagagtgcaagttgttctacctggcgggccaggacaccaccgccagcctcttagtgtggacccttatcatgctcgccaaacatcagaattggcagcagcttgcacgccaggagatattcaacgccttcggagacaagataccccagttccacgacctcaaccacctcaaaatcgtcaacatgatcctgcaagaggtgttgcgactctacccacccgtcacggagactaaccgcgaagtcattcaagacatcaagctcgggaatatgatcctgccagccggagtggtggtcaacatcccgatactgctgatgcaccacgacgagaaaatctggggaaacgacgcgaaagagttcaaccccgaacgattctctgagggtatcagtaaggcgtccaaagggaatatgtcacttttcgccttcggctggggaccacgcatctgcatcggctccaacttcgcaatgatcgaggcgaagctcacgctagtgtgcatcctgcaacggttcgtcttcgagctgtcaccctcctaccttcacgccccaagcgcgattggctccttgaggccgcagtttggcgcaccgatcatcttccgaccccgaatatga

SEQ ID NO: 12 - DNA, 1470 bp, synthetic construct
  source 1..1470: mol_type = other DNA
  misc_feature 1..1470: note = Artificial sequence
atggagatccttttcacctatctatccatcttcgctacgttcttcttcctctacaaaatcatccaaagtctaaagcccaacaacaagaagcacccgccttcaccgccgagctaccccttgctggggcatctgcacttgttaaaacctccattccatcggacactgcaatccctcgcccaacgctacggacccatcttcacgctaaagctcgggttgcaaagggctgtcgtcgtttcgtcggcatgggccgccgaggaatgcttcggtcaaaacgatgtggtctttgccaaccgtccgaagttcattatcggacaacacctcggttacaaccactccattctaatctggtcgccgtacggggactattggcggaacttgaggcgtgtgacgacgatcactatgctgtcgatgcgccgcatcaacgaggcgggcccaacaaggaagctggaaatacggaacatgattatgaacttacttgaatctggctccggcacccagaaggtgaacatgaacgaggttctctcaaaacttgcacgcaacttcgtgatgagaatagtgaacgggaaaagttgggagaaaatgattatcaagcctcccgagaacttgatgaccatctgcgatttcctaccattccttagatgggttgacttcaagggtatcgagaaggatatgaaggagaaacaagtggagcgagacgagttcctgcaatcgctggttgacgagttccgggagagtaagaagaagggcgaggacttggggatgaacaacaccctgattgagcagttgctggaccttgaacaagcggagcctgaccaatactcggatgagacgatcaaggggatcatcctcgtgatgctattagctggatcggagactacggcccgcacgctggagtgggcgcttagcaacttgataaaccatccggagatcctggcaaaagctaggaccgagattgacctgcacgtgggcaaggagcgcctagtggacgattccgacctcccaaaacttacatacacccgctgcatagtgtacgagaccctccggttgttcccagcagcccctttactggttccgcactttagtagtcaagactgtacaatcggcggttaccacgtcccgaaggggacgatgctgttcgtgaacgcatgggccattcaccgcgatcctactctgtgggacgagcccaccgtctttaaacctgagagattcgagaaggagaaggaagggttcaagttcatgccctttgggattgggcgaagatcatgtcctggcaacaacgtggcactgaggaacgtcagccttacgctcgctactctgatccaatgcttcgactgggaggccgcagagagcggatcaattgaccttaccgagaagcgtggggccggggctatcatagtgccgaaggagaagccgctcgaagcgatttgccgcccacggccttcaatggaggacttcctcgccaaaatctag

SEQ ID NO: 13 - DNA, 1470 bp, synthetic construct
  source 1..1470: mol_type = other DNA
  misc_feature 1..1470: note = Artificial sequence
atggagatactctttacctacctgtccatattcgccacattcttcttcctctacaagataattcaaagcctcaagacgaacaacaagaagcacccaccaagtccgccatcctaccctctcctgggacatctgcacttgctgaaacctccgtttcaccgaacgttgcaatcactcgcgcagcgctatggacccatcttcacacttaagctcggactacaaagagcggtggtagtgtcctctgcctgggccgcagaggagtgcttcggccagaatgacgtggtattcgccaatcggccaaagttcatcattggtcaacatcttggctacaaccacagcatattgatatggagtccctatggagactactggaggaacctgaggagggtgaccacaataaccatgctaagtatgcgacggatcaacgaggccgggccgacccgaaaactggagataaggaacatgattatgaacctcttggagtcgggctccggcacccagaaggtgaacatgaacgaggtgctctcgaagctggcacgcaacttcgtgatgagaatagtaaacgggaagtcctgggagaaaatgattatcaagcctcctgagaatctaatgacgatttgcgactttctgcctttcctacgctgggttgacttcaaggggattgagaaggacatgaaggagaagcaagtcgaacgcgacgagttcttgcaaagcctcgtggacgagttccgggagagcaagaagaagggcgaggatctcggcatcaacaacacgcttatcgagcagctactggacctgcaacaagcggagccggaccaatactccgatgagaccatcaaggggatcattctcgtgatgctactcgctggttctgagacgacagcccgaacgcttgaatgggctctgtcgaatctcatcaaccaccccgaaatcttggtcaaggcccggacggagattgatctgcacgtgggaaaggagcgcctcgtggacgatagtgacctacccaagctaacttacacgcgctgcatcgtctatgagaccctgaggctgtttcccgccgcccctctccttgtgccacacttctctagtcaagactgcacaatcggcggctaccatgtgccgaaagggacgatgctattcgttaatgcttgggctatccaccgagacccaaccctttgggatgagcccaccgtcttcaagccggagcgtttcgagaaggagaaggaggggttcaaattcatgccgtttgggatcggacgccgttcatgccctggcaacaacgtagccctaaggaatgtgagcctaaccctagcaactctcatacaatgctttgactgggaggctgcggagtcggggtcgattgatcttacggagaagcgaggagtaggtgctatcatcgtgccgaaggagaagccgttagaggcaatctgccgcccacggccatcaatggaggactttctggcaaagatatga

SEQ ID NO: 14 - DNA, 1008 bp, Arabidopsis thaliana
  source 1..1008: mol_type = genomic DNA
tttattagagtagattagaatcttttatgccaagtattgataaattaaatcaagaagataaactatcataatcaacatgaaattaaaagaaaaatctcatatatagtattagtattctctatatatattatgattgcttattcttaatgggttgggttaaccaagacatagtcttaatggaaagaatcttttttgaactttttccttattgattaaattcttctatagaaaagaaagaaattatttgaggaaaagtatatacaaaaagaaaaatagaaaaatgtcagtgaagcagatgtaatggatgacctaatccaaccaccaccataggatgtttctacttgagtcggtcttttaaaaacgcacggtggaaaatatgacacgtatcatatgattccttcctttagtttcgtgataataatcctcaactgatatcttcctttttttgttttggctaaagatattttattctcattaatagaaaagacggttttgggcttttggtttgcgatataaagaagaccttcgtgtggaagataataattcatcctttcgtctttttctgactcttcaatctctcccaaagcctaaagcgatctctgcaaatctctcgcgactctctctttcaaggtatattttctgattctttttgtttttgattcgtatctgatctccaatttttgttatgtggattattgaatcttttgtataaattgcttttgacaatattgttcgtttcgtcaatccagcttctaaattttgtcctgattactaagatatcgattcgtagtgtttacatctgtgtaatttcttgcttgattgtgaaattaggattttcaaggacgatctattcaatttttgtgttttctttgttcgattctctctgttttaggtttcttatgtttagatccgtttctctttggtgttgttttgatttctcttacggcttttgatttggtatatgttcgctgattggtttctacttgttctattgttttatttcagggt

SEQ ID NO: 15 - DNA, 289 bp, Medicago truncatula
  source 1..289: mol_type = genomic DNA
gggtcttagtttacttttcttgatttgtaatgtacaatggggctgtggtgtttatatttcatttaatcgttaatgtactactttatactagttgtttatatttaaaaaagcataaactttgcctcatctaaatgtacttgaattgagtttacttagaagtgcttgaattgagtttgtgtaaatgaacataattttagtagtgcttagattaagttcacatatcactactttataccaggggattactattctttttagcttttgtttttactgttggttttttgccaaa

SEQ ID NO: 16 - protein, 492 aa, Amaranthus palmeri
MEILFTYLSIFATFFFLYKIIQSLKPNNKKHPPSPPSPPSYPLLGHLHLLKPPFHRTLQSLAQRYGPIFTLKLGLQRAVVVSSAWAAEECFGQNDVVFANRPKFIIGQHLGYNHSILIWSPYGDYWRNLRRVTTITMLSMRRINEAGPTRKLEIRNMIMNLLESGSGTQKVNMNEVLSKLARNFVMRIVNGKSWEKMIIKPPENLMTICDFLPFLRWVDFKGIEKDMKEKQVERDEFLQSLVDEFRESKKKGEDLGMNNTLIEQLLDLQQAEPDQYSDETIKGIILVMLLAGSETTARTLEWALSNLINHPEILAKARTEIDLHVGKERLVDDSDLPKLTYTRCIVYETLRLFPAAPLLVPHFSSQDCTIGGYHVPKGTMLFVNAWAIHRDPTLWDEPTVFKPERFEKEKEGFKFMPFGIGRRSCPGNNVALRNVSLTLATLIQCFDWEAAESGSIDLTEKRGVGAIIVPKEKPLEAICRPRPSMEDFLAKI

SEQ ID NO: 17 - protein, 495 aa, Amaranthus palmeri
MEILFTFLSIFATFFFLYKIIQSLKPNNKKHPPSPPSPPSPPSYPLLGHLHLLKPPFHRTLQSLAQRYGPIFTLKLGLQRAVVVSSAWAAEECFGQNDVVFANRPKFIIGQHLGYNHSILIWSPYGDYWRNLRRVTTITMLSMRRINEAGPTRKLEIRNMIMNLLESGSGTQKVNMNEVLSKLARNFVMRIVNGKSWEKMIIKPPENLMTICDFLPFLRWVDFKGIEKDMKEKQVERDEFLQSLVDEFRESKKKGEDLGMNNTLIEQLLDLQQAEPDQYSDETIKGIILVMLLAGSETTARTLEWALSNLINHPEILAKARTEIDLHVGKERLVDDSDLPKLTYTRCIVYETLRLFPAAPLLVPHFSSQDCTIGGYHVPKGTMLFVNAWAIHRDPTLWDEPTVFKPERFEKEKEGFKFMPFGIGRRSCPGNNVALRNVSLTLATLIQCFDWEAAESGSIDLTEKRGAGAIIVPKEKPLEAICRPRPSMEDFLAKI

SEQ ID NO: 18 - DNA, 1479 bp, Amaranthus palmeri
  source 1..1479: mol_type = other DNA
  CDS 1..1479: note = Amaranthus palmeri CDS encoding SEQ ID NO:16.
atggaaattttattcacttatttatccatctttgctacttttttcttcctctataaaatcatccaatcccttaaaccaaacaataaaaaacacccaccaagcccaccaagcccaccatcatacccattattgggccacttacaccttctaaaacccccattccaccgtacactccaatctctagcccaacgttacggcccaatcttcactctcaaattgggccttcaacgggccgtggtagtttcctccgcatgggccgccgaagaatgtttcggtcaaaacgacgtcgtttttgcaaacagaccaaaattcataataggacaacatttaggatacaaccactctatccttatctggtccccatatggggactactggcggaacctccgacgtgtcacaaccattaccatgttatccatgagacggatcaacgaagcgggtccgacccggaaacttgagatccgaaacatgataatgaaccttctagaaagtgggagtgggacccaaaaggtgaatatgaatgaagtgctgagtaaactagcaagaaattttgtaatgaggatagtaaatggaaaatcatgggaaaaaatgataataaaaccccctgaaaatttaatgactatttgtgattttttaccatttttaagatgggtagattttaaagggatagaaaaggatatgaaggagaaacaggttgaaagagatgagtttttacagagtttggttgatgaatttagagaaagtaagaagaaaggtgaggatttagggatgaataatactttgattgaacaattgctggatttgcaacaagctgaacctgatcaatactctgatgaaactatcaaaggaatcattttggtaatgttactagcgggatcagaaaccactgcacgaaccttagaatgggcattatcaaatctcattaaccacccagaaatcctcgcgaaagcaagaacagaaatcgacctccatgtaggcaaggaacgactagtagatgattctgatctcccaaaattaacgtatacacgttgcattgtatacgaaactctaagactatttccggctgcaccacttttagtacctcatttttcttcgcaagattgcaccataggagggtatcacgtaccaaaaggtacaatgcttttcgtaaatgcttgggctatacacagagacccgaccctatgggacgagcctactgtgtttaagcccgagaggttcgaaaaggagaaagaagggtttaagtttatgccctttggaattggaaggagatcttgtccagggaacaatgtggctcttaggaatgtttcattgacattggctactcttattcagtgctttgattgggaagcagccgagagtggatcaattgatctaaccgaaaagcgtggtgttggtgcgatcattgtccctaaggaaaagccattggaggctatatgtcgaccacgaccctcaatggaggatttcctcgctaaaatttag

SEQ ID NO: 19 - DNA, 1485 bp, Amaranthus palmeri
  source 1..1485: mol_type = other DNA
  CDS 1..1485: note = Amaranthus palmeri CDS encoding SEQ ID NO:17.
atggaaattttattcacttttttatccatctttgctacttttttcttcctctataaaatcatccaatccctcaaaccaaacaacaaaaaacacccaccaagcccaccaagcccaccaagcccaccatcatacccattattgggccacttacaccttctaaaacccccattccaccgtacactccaatctctagcccaacgttacggcccaatcttcactctcaaattgggccttcaacgggccgtggtagtttcctccgcatgggccgccgaagaatgtttcggtcaaaacgacgtcgtttttgcaaacagaccaaaattcataataggacaacatttaggatacaaccactctatccttatctggtccccatatggggactactggcggaacctccgacgtgtcacaaccattaccatgttatccatgagacggatcaacgaagcgggtccgacccggaaacttgagatccgaaacatgataatgaaccttctagaaagtgggagtgggacccaaaaggtgaatatgaatgaagtgctgagtaaacttgcaagaaattttgtaatgaggatagtaaatggaaaatcatgggaaaaaatgattataaaaccccctgaaaatttaatgactatttgtgattttttaccatttttaagatgggtagattttaaagggatagaaaaggatatgaaggaaaaacaggttgaaagagatgagtttttacaaagtttggttgatgaatttagagaaagtaagaagaaaggtgaggatttagggatgaataatactttgattgaacaattgctggatttgcaacaagctgaacctgatcaatactctgatgaaactatcaaaggaatcattttggtaatgttactagcgggatcagaaaccacagcacgaaccttagaatgggcactatcaaatctcattaaccacccagaaatcctcgcgaaagcaagaacagaaatcgacctccatgtaggcaaggaacgactagtagatgattctgatctaccaaaattaacgtatacacgttgcattgtatacgaaactctaagactatttccggctgcaccacttttagtgcctcatttttcttcacaagattgcaccataggagggtatcacgtaccaaaagggacgatgcttttcgtaaatgcttgggctatacacagagacccgaccctatgggacgagcctactgtgtttaagcccgagaggttcgaaaaggagaaagaagggtttaagtttatgccctttggaattggaaggagatcttgtccaggaaataatgtggctcttaggaatgtttcattgacattggctactcttattcagtgctttgattgggaagcagccgagagtggatcaattgatctaaccgaaaagcgtggtgctggtgcgatcattgtccctaaggaaaagccattggaggctatatgtcgaccacgaccctcaatggaggatttcctcgctaaaatt

SEQ ID NO: 20 - DNA, 1479 bp, synthetic construct
  source 1..1479: mol_type = other DNA
  misc_feature 1..1479: note = Artificial sequence
atggaaatcttgttcacctacctatctatcttcgctacattcttcttcctgtacaaaatcatacaatcgctcaagccaaacaacaagaagcacccgccgtcccctccatccccaccctcttacccgctgcttgggcaccttcacctcttgaagcctccgttccaccgaaccttgcagtccctcgctcaaagatacggcccaatcttcacgcttaagttggggcttcaacgcgccgtggtcgttagctcggcgtgggcggccgaggagtgctttggccagaacgacgtggtattcgctaatcgccccaagttcattatcggacagcatcttggatacaaccacagcatcctaatctggtcgccctacggggattactggcgtaacctacgcagagttaccactattactatgctgagtatgcggaggatcaacgaggccgggcccacacgcaagctggagatccgaaacatgattatgaacctactcgaatctggaagtggaacccagaaggtcaacatgaacgaggtgctctcgaagttggctaggaacttcgtgatgcggattgtcaacggcaagtcgtgggagaaaatgattatcaagccgccggagaacctgatgacgatttgcgacttccttccattcttgagatgggttgacttcaaaggaattgagaaggacatgaaggagaagcaagtggagcgagacgagttcttacaatcccttgtcgatgagtttcgggagagcaagaagaagggcgaagacctggggatgaacaacaccctaatcgagcaacttctcgacctgcaacaagccgagcctgaccagtattctgatgagaccatcaaggggattatcttggtcatgcttctggcggggagcgagacgacagctagaacactagagtgggcgctttccaaccttatcaatcacccggagattctagcaaaggctcggactgaaatcgaccttcacgtcgggaaggagcgccttgtggacgacagcgaccttccaaagctgacatacacacggtgcatcgtgtacgagaccctgcgactattcccagcggccccactgctagtgccccacttctcatcacaagattgcacgattggaggttaccatgtcccgaagggtacgatgctgttcgtcaacgcctgggccatacaccgagatccaacactatgggacgaaccgaccgtcttcaagccggaaaggttcgagaaggagaaggaggggttcaagttcatgccgttcgggatcggtcgtcgtagttgcccagggaacaacgttgcgctgagaaacgtctcccttacactagcgacgcttattcaatgtttcgactgggaggccgcagagtccggtagtatcgacctgactgagaagcgaggcgttggtgccataatcgttcctaaggaaaagccccttgaggctatatgccgtccgaggcccagcatggaagacttcctcgccaagatatga

SEQ ID NO: 21 - DNA, 1488 bp, synthetic construct
  source 1..1488: mol_type = other DNA
  misc_feature 1..1488: note = Artificial sequence
atggagattctgtttaccttcctttcgatcttcgccacgttcttcttcctctacaagattatccaatcactgaaacccaacaacaagaagcacccaccctccccgccctccccaccatcaccgccctcataccccttgctcggtcacttacacttgctcaagcctccgttccatcggacacttcaaagcctggcgcagagatatgggcctatcttcacgttgaagttggggctccaacgcgccgtcgtcgtgtcatccgcgtgggccgccgaggaatgctttggacaaaatgacgtcgttttcgccaaccgcccgaagttcatcatcggccagcatctcggttacaaccactccatcctgatctggtcaccttatggagattactggcgcaacctgcggcgcgtcaccacaatcacgatgctttctatgcggcgtattaacgaggccgggccaactcggaagctggagattcgtaacatgattatgaacctattggagtccggaagcggaacccagaaggtcaacatgaacgaggttctgagtaaactggcgaggaacttcgtgatgaggatcgtcaacggcaagagttgggagaaaatgattatcaagcctcccgagaaccttatgacaatatgcgactttctgcctttcctgcgctgggttgacttcaaggggattgagaaggatatgaaggagaagcaagtcgaaagggatgagttcttacaaagcctcgttgatgagtttcgagagtccaagaagaagggcgaggatcttgggatgaacaacacattgatcgagcagctcctcgatctacaacaagccgagccagaccaatactccgacgagaccatcaagggcatcattcttgtcatgcttctcgcggggagtgagacaacggcgaggactctggagtgggccctctctaatcttatcaaccaccccgagattctggctaaggcgcgaactgagattgatcttcacgttggaaaggaaagattggtggatgactcggatctcccgaagctcacatacacccggtgcatcgtgtacgagacgctgcgcctattcccggctgctccgctgttagtacctcacttctcatcacaagactgtacaattggaggctaccacgttccgaagggtactatgcttttcgtgaacgcctgggcgatccaccgcgatccaacactgtgggacgaacctaccgtgtttaagccggagcgattcgagaaggagaaggaggggtttaagttcatgcccttcgggatcggtcgtcgatcctgcccaggaaacaacgtggccctacgaaacgtgagcttgacgcttgctaccttgatccaatgcttcgactgggaagcagcggaatctggatctatcgaccttactgagaaacgaggcgctggggcaatcatcgtgccgaaggagaagcctctcgaagcgatctgtcggccgcgtcctagcatggaggactttctcgccaagatatag

SEQ ID NO: 22 - DNA, 1488 bp, synthetic construct
  source 1..1488: mol_type = other DNA
  misc_feature 1..1488: note = Artificial sequence
atggagatcctgttcacgttcctctccatcttcgcaacattcttcttcctgtacaagattatccaatccctgaaaccgaacaacaagaagcacccaccgtccccgccttccccaccttcaccaccgtcttacccgctactcgggcacctccacctccttaagccgcccttccaccgcactctccaatcgctcgcccagcgatacgggcccatcttcactcttaagctagggctccaacgtgcggtcgtagtcagctcggcctgggcggccgaggagtgcttcgggcaaaatgacgtcgtgttcgccaaccggccaaagttcattatcggccagcacctcggctacaaccactcgatcctaatctggtcgccgtacggagactattggcgcaacctgcgccgcgtgacgacgattaccatgctttccatgcgccgcatcaatgaggccggaccaacgcgcaagctggagattcgcaacatgattatgaacctgctggagtctggcagcggcacccagaaggtgaacatgaacgaggtcttaagcaagctggcccgcaatttcgtcatgcggatagtgaacggcaaatcgtgggagaaaatgattatcaagcctccggagaacctcatgacgatctgtgacttccttccgttcctgagatgggttgacttcaaagggatcgagaaggacatgaaggaaaagcaagtggagcgggacgagttcctccaatccctcgttgacgagttccgcgagagcaagaagaagggcgaagacctggggatgaacaacaccttaatcgagcagctcttggacctccaacaagccgagcccgaccaatactctgacgagaccatcaagggcatcatacttgtcatgcttctggcgggctctgaaaccaccgcgcgcactctggagtgggcgctttcgaacctcatcaaccatccggagatccttgcaaaggctcgaaccgagatcgacctgcatgttgggaaggagcgcctggtggacgactctgacctgccgaagctgacctacacaaggtgcatcgtgtacgagactctgcgacttttcccggcggccccgttgcttgtgccccacttctcctcccaagactgcaccattggcggctaccacgtgccaaaagggaccatgctgttcgtgaacgcatgggccatccatcgtgacccaacgttgtgggacgagccgacggtcttcaagcctgagcgctttgagaaggagaaggagggcttcaagttcatgccgttcgggataggccgtcgatcctgccctgggaacaatgtcgccctccgcaatgtgtccctgaccctagccaccctcatccaatgctttgactgggaggcggccgagtcggggtctattgatctgaccgagaaacgaggtgcgggagcgatcattgtcccgaaggagaagcccttagaggcaatctgccgcccgcgaccgagcatggaggacttcctcgccaaaatctag

SEQ ID NO: 23 - protein, 491 aa, Amaranthus tuberculatus
MEILYTYLSIFATIFFLYKIIQSLKPNNKKLPPSPPSYPILGHLHLLKPPFHRTLQSLAQRYGPIFSLQLGLQRALVVSSAWAAEECFSQNDVVFANRPKFIIGQHLGYNHSILIWSPYGDYWRNLRRVTTITMLSMRRINEAGPTRKLEIRNMIMDLLETSKGGSGTHKVNMNEVLSKLARNFVMRIVNGKSWEKMIIKPPENLMTICDFLPFLRWVDFKGIEKDMKEKQIERDEFLQSLVDEIRESRKKGEDLGMNTLIEQLLDLQHAEPDQYSDETIKGIILVMLLAGSETTARTLEWALSNLINHPEILAKARTEIDLHVGKERLVDDSDLPKLTYTRCIVYETLRLFPVAPLLVPHFSSQDCTIGGYHVPKGTMLFVNAWAIHRDPTLWDEPTVFKPERFEKEKEGFKFMPFGIGRRSCPGNNVALRNVSLTLATLIQCFDWEASESGSIDLTEKRGAGAIIVPKEKPLEAICRPRPSMEDFLAKI

SEQ ID NO: 24 - DNA, 1476 bp, Amaranthus tuberculatus
  source 1..1476: mol_type = other DNA
  CDS 1..1476: note = Amaranthus tuberculatus CDS encoding SEQ ID NO:23.
atggaaatattatacacttatttatccatatttgctactattttcttcctctataaaatcattcaatcccttaaaccaaacaacaaaaaactcccaccaagcccaccatcatacccaatattgggccacttacaccttctaaaacccccattccaccgtacactccaatctctagcccaacgttacggcccaatcttctctctccaattgggccttcaacgggccttggtagtttcctccgcatgggccgccgaagaatgtttcagtcaaaacgacgtcgtttttgcaaacaggcctaaattcataataggacaacatttaggatacaaccactctatccttatctggtccccttatggggactactggcggaacctccgacgtgttacaaccattaccatgttatccatgagacggatcaacgaagcgggtccgacccggaaacttgagatccgaaacatgataatggaccttctagaaacctcaaaaggtgggagtgggacccacaaagtgaatatgaatgaagtacttagtaaacttgcaagaaattttgtaatgaggatagtaaatggaaaatcatgggaaaaaatgattataaaaccccctgaaaatttgatgactatttgtgattttttgccatttttaagatgggttgattttaaagggatagagaaggatatgaaggagaaacagattgaaagagatgagtttttacaaagtttggttgatgaaattagagaaagtaggaagaaaggtgaggatttagggatgaatactttgattgaacagttgctggatttgcaacatgctgagcctgatcaatactctgatgaaactatcaaaggaatcattttggtaatgttactagcgggatcagaaaccacagcacgaaccttagaatgggcattatcaaatctcattaaccacccagaaatcctcgcaaaagcaagaacagaaatcgacctccatgtaggcaaggaacgactagtagatgattctgatctaccaaaattaacgtatacacgttgcattgtatacgaaactctaagactatttccggttgcaccacttttagtacctcatttttcgtcacaagattgcaccataggagggtatcacgtaccaaaagggacaatgcttttcgtaaatgcttgggctatacacagagacccgaccctatgggacgagcctactgtgtttaagcccgagaggttcgaaaaggagaaagaagggtttaagtttatgccctttggaattggaaggagatcttgtccggggaataatgtggctcttaggaacgtttcattgacattggctactcttattcagtgctttgattgggaagcatctgagagtggatcaattgatctaaccgaaaagcgtggtgctggtgctatcattgtccctaaggaaaagccattagaggctatatgtcgacctcgaccttcaatggaggatttcctcgctaaaatttag

SEQ ID NO: 25 - DNA, 1476 bp, synthetic construct
  source 1..1476: mol_type = other DNA
  misc_feature 1..1476: note = Artificial sequence
atggaaatcctttacacttacttgtcgatattcgccaccatattctttctatacaagataatccaaagccttaagcccaacaataagaagctaccaccgagcccgccgtcctacccgatcctcggacacctccacctactcaaaccgcctttccaccgtacgttgcaatcccttgcccaacgttacggacccatattctctctacaactggggttacaacgtgccctggtggtgtcttcggcctgggcggccgaggagtgcttctcgcagaacgatgtcgtgttcgctaaccgcccaaagttcatcatcggccaacacctcggctacaaccacagcatacttatctggtcaccctacggcgattactggcgcaacctccgccgagtaaccactatcacgatgctatccatgcgccgcattaacgaggccggtcctacgcgaaagctggagataaggaatatgattatggatctgttggaaacgtccaaaggcgggtctggcactcacaaggtcaacatgaacgaggtgctcagcaagctcgcgcgcaacttcgtcatgagaatagtgaacgggaagtcgtgggagaaaatgattatcaagcctccggagaacctgatgacaatttgcgacttcctccccttcctccgttgggttgacttcaagggcatcgagaaggacatgaaggaaaagcaaattgagcgagacgagttcctccaaagcctcgtggacgagatacgcgaatcgcgcaagaagggcgaggatcttgggatgaacacccttattgagcagctcttagacctccaacacgccgaacccgatcaatactctgacgagaccatcaagggcataatcctcgtcatgttgcttgccgggagtgagaccacagcccgtacccttgagtgggcactctcgaaccttattaaccacccggaaattctggccaaagcccgcacggagatagacctccatgtggggaaggagagacttgtggacgactcggacctgcccaaattgacctacacgcgctgcatagtctatgagacattgagactattccctgtggccccattacttgtgccgcacttctctagtcaagactgcactattggcggctaccatgtgccaaaggggaccatgctattcgtgaatgcttgggccatccatcgagacccaacactgtgggatgagcctacggtcttcaagccggagagattcgagaaggagaaggagggcttcaaattcatgcctttcggaatcggtcggcgctcctgccctggcaacaacgtggctctacgaaatgtttccctcactcttgctaccctaatccaatgcttcgattgggaggcgagtgaatctgggtccattgacctgacggagaaacgaggtgcgggtgctatcatcgtgcctaaggagaaaccgcttgaggcaatctgtcggcctcgaccctcaatggaagacttcctggccaagatatga

SEQ ID NO: 26 - protein, 491 aa, Amaranthus cruentus
MEILYTYLSIFATFFFLYKIIQSLKPNNKKHPPSPPSYPILGHLHLLKPPFHRTLQSLAQRYGPIFSLKLGLQRALVVSSAWAAEECFSQNDVVFANRPKFIVGQHLGYNHSILIWSPYGDYWRNLRRVTTITMLSMRRINEAGPTRKVEIRNMIMDLLESSNFGSGTQKVNMNEVLSKLARNFVMRIVNGKSWEKMIIKPPENLMNICDFLPFLRWVDFKGIEKDLKEKQVERDEFLQSLVDEIRESRKKGEDLGMNTLIEQLLDLQQAEPDQYSDETIKGIILVMLLAGSETTARTLEWALSNLINHPEILVKARTEIDLHVGKERLVDDSDLPKLTYTRCIVYETLRLFPAAPLLVPHFSSQDCTIGGYHVPKGTMLFVNAWAIHRDPTLWDEPTVFKPERFEKEKEGFKFMPFGIGRRSCPGNNVALRNVSLTLATLIQCFDWEAAQSGSIDLTEKRGAGAIIVPKEKPLEAICRPRPSMEDFLAKI

SEQ ID NO: 27 - protein, 489 aa, Amaranthus palmeri
MEILFTFLSIFATFFFLYKIIQSLKTNNKKHPPSPPSYPLLGHLHLLKPPFHRTLQSLAQRYGPIFTLKLGLQRAVVVSSAWAAEECFGQNDVVFANRPKFIIGQHLGYNHSILIWSPYGDYWRNLRRVTTITMLSMRRINEAGPTRKLEIRNMIMNLLESGSGTQKVNMNEVLSKLARNFVMRIVNGKSWEKMIIKPPENLMTICDFLPFLRWVDFKGIEKDMKEKQVERDEFLQSLVDEFRESKKKGEDLGINNTLIEQLLDLQQAEPDQYSDETIKGIILVMLLAGSETTARTLEWALSNLINHPEILVKARTEIDLHVGKERLVDDSDLPKLTYTRCIVYETLRLFPAAPLLVPHFSSQDCTIGGYHVPKGTMLFVNAWAIHRDPTLWDEPTVFKPERFEKEKEGFKFMPFGIGRRSCPGNNVALRNVSLTLATLIQCFDWEAAESGSIDLTEKRGVGAIIVPKEKPLEAICRPRPSMEDFLAKI

SEQ ID NO: 28 - protein, 492 aa, Amaranthus palmeri
MEILFTFLSIFATFFFLYKIIHSLKTNNKKHPPSPPSPPSYPLLGHLHLLKPPFHRTLQSLAQRYGPIFTLKLGLQRAVVVSSAWAAEECFGQNDVVFANRPKFIIGQHLGYNHSILIWSPYGDYWRNLRRVTTITMLSMRRINEAGPTRKLEIRNMIRNLLESGSGTQKVNMNEVLSKLARNFVMRIVNGKSWEKMIIKPPENLMTICDFLPFLRWVDFKGIEKDMKEKQVERDEFLQSLVDEFRESKKKGEDLGMNNTLIEQLLDLQQAEPDQYSDETIKGIILVMLLAGSETTARTLEWALSNLINHPEILAKARTEIDLHVGKERLVDDSDLPKLTYTRCIVYETLRLFPAAPLLVPHFSSQDCTIGGYHVPKGTMLFVNAWAIHRDPTLWDEPTLFKPERFEKEKEGFKFMPFGIGRRSCPGNNVALRNVSLTLATLIQCFDWEAAESGSIDLTEKRGVGAIIVPKEKPLEAICRPRPSMEDFLAKI

SEQ ID NO: 29 - DNA, 1476 bp, Amaranthus cruentus
  source 1..1476: mol_type = genomic DNA
atggaaatattatacacttatttatccatctttgctacttttttcttcctttataaaatcattcaatcccttaaaccaaacaacaaaaaacacccaccaagcccaccatcatacccaatattgggccacttacaccttctaaaacccccattccaccgtacactccaatctctagcccaacgttacggcccaatcttctctctcaaattgggccttcaacgggccttggtagtttcctcggcatgggccgccgaagaatgtttcagtcaaaacgacgtcgtttttgcgaacagaccaaaatttattgtaggacaacatctaggatacaaccactctatccttatctggtccccatatggggactactggcggaacctccgacgtgttacaaccattaccatgttatccatgagacggatcaacgaagcgggtccgacccggaaagttgagatccgaaacatgataatggaccttctagaaagttccaactttgggagtgggacccaaaaggtgaatatgaatgaagtgcttagtaaacttgcaagaaattttgtaatgagaatagtaaatggaaaatcatgggaaaaaatgattataaaaccacctgaaaatttgatgaatatttgtgattttttgccatttttaagatgggtggattttaaagggatagaaaaggatttgaaggagaaacaggttgaaagagatgagtttttacaaagtttggttgatgaaattagagaaagtaggaagaaaggtgaggatttagggatgaatactttgattgaacaattgctggatttgcaacaagctgaacctgatcaatactctgatgaaactatcaaaggaatcattttggtaatgttactagcgggatcagaaaccacagcacgaaccttagaatgggcattatcaaatctcattaaccaccctgaaatcctcgtgaaagcaagaacagaaatcgacctacatgtaggcaaggaacgactagtagatgattctgatctaccaaaattaacgtatacacgttgcattgtatacgaaactttaagactatttccggctgcaccacttttagtgcctcatttttcttcacaagattgcaccataggagggtatcacgtaccaaaagggacaatgcttttcgtaaatgcttgggctatacacagagacccgaccctatgggacgagcctactgtttttaagcccgagaggtttgaaaaggagaaagaagggtttaagtttatgccctttggaattggaaggagatcttgtccagggaataatgtggctcttaggaatgtttcattgacattggctactcttattcagtgctttgattgggaagcagctcagagtggatcaattgatctaaccgaaaagcgtggtgctggtgctatcattgtccctaaggaaaagccattggaggctatatgtcgaccacgaccctcaatggaggatttcctcgctaaaatttag

SEQ ID NO: 30 - DNA, 1470 bp, Amaranthus palmeri
  source 1..1470: mol_type = genomic DNA
atggaaattttattcacttttttatccatctttgctacttttttcttcctctataaaatcatccaatcccttaaaacaaacaataaaaaacacccaccaagcccaccatcatacccattattgggccacttacaccttctaaaacccccattccaccgtacactccaatctctagcccaacgttacggcccaatcttcactctcaaattgggccttcaacgggccgtggtagtttcctccgcatgggccgccgaagaatgtttcggtcaaaacgacgtcgtttttgcaaacagaccaaaattcataataggacaacatttaggatacaaccactctatccttatctggtccccatatggggactactggcggaacctccgacgtgtcacaaccattaccatgttatccatgagacggatcaacgaagcgggtccgacccggaaacttgagatccgaaacatgataatgaaccttctagaaagtggaagtgggacccaaaaggtgaatatgaatgaagtgctgagtaaactagcaagaaattttgtaatgaggatagtaaatggaaaatcatgggaaaaaatgataataaaaccccctgaaaatttaatgactatttgtgattttttaccatttttaagatgggtagattttaaagggatagaaaaggatatgaaggaaaaacaggttgaaagagatgagtttttacaaagtttggttgatgaatttagagaaagtaagaagaaaggtgaggatttagggataaataatactttgattgaacaattgctggatttgcaacaagctgaacctgatcaatactctgatgaaactatcaaaggaatcattttggtaatgttactagcaggatcagaaaccacagcacgaaccttagaatgggcattatcaaatctcataaaccacccagaaatcctcgtgaaagcaagaacagaaatcgacctccatgtaggcaaggaacgactagtagatgattctgatctaccaaaattaacgtatacacgttgcattgtatacgaaactctaagactatttccggctgcaccacttttagtacctcatttttcttcgcaagattgcaccataggaggctatcacgtaccaaaagggaccatgcttttcgtaaatgcttgggctatacacagagacccgaccctatgggacgagcctactgtgtttaagcctgagaggttcgagaaggagaaagaagggtttaagtttatgccctttggaattggaaggagatcttgtccagggaacaatgtggctcttaggaacgtttcattgacattggctactcttattcagtgctttgattgggaagcagctgagagtggatcaattgatctaaccgaaaagcgtggtgttggtgcgatcattgtccctaaggaaaagccattggaggctatatgtcgaccacgaccctcaatggaggatttcctcgctaaaatttag

SEQ ID NO: 31 - DNA, 1479 bp, Amaranthus palmeri
  source 1..1479: mol_type = genomic DNA
atggaaattttattcacttttttatccatctttgctacttttttcttcctctataaaatcatccattcccttaaaacaaacaacaaaaaacacccaccaagcccaccaagcccaccatcatacccattattgggccacttacaccttctaaaacccccattccaccgtacactccaatctctagcccaacgttacggcccaatcttcactctcaaattgggccttcaacgggccgtggtagtttcctccgcatgggccgccgaagaatgtttcggccaaaacgacgtcgtttttgcaaacagaccaaaattcataataggacaacatttaggatacaaccactctatccttatctggtccccatacggcgactactggcggaacctccgacgtgtcacaaccattaccatgttatccatgagacggatcaacgaagcgggtccgacccggaaacttgagatccgaaacatgataaggaaccttctagaaagtgggagtgggacccaaaaggtgaatatgaatgaagtgctgagtaaactagcaagaaattttgtaatgaggatagtaaatggaaaatcatgggaaaaaatgataataaaaccccctgaaaatttaatgactatttgtgattttttaccatttttaagatgggtagattttaaagggatagaaaaggatatgaaggaaaaacaggttgaaagagatgagtttttacaaagtttggttgatgaatttagagaaagtaagaagaaaggtgaggatttagggatgaataatactttgattgaacaattgctggatttgcaacaagctgaacctgatcaatactctgatgaaactatcaaagggatcattttggtaatgttactagcgggatcagaaaccacagcacgaaccttagaatgggcattatcaaatctcattaaccacccagaaatcctcgcgaaagcaagaacagaaatcgacctccatgtaggcaaggaaagactagtagatgattctgatctcccaaaattaacgtatacacgttgcattgtatacgaaactctaagactatttccggctgcaccacttttagtacctcatttttcttcacaagattgcaccataggaggctatcacgtaccaaaagggacaatgcttttcgtaaatgcttgggctatacacagagacccgaccctatgggacgagcctactttgtttaagcccgagaggttcgaaaaggagaaagaagggtttaagtttatgccctttggaattggaaggagatcttgtccagggaacaatgtggctcttaggaacgtttcattgacattggctactcttattcagtgctttgattgggaagcagctgagagtggatcaattgatctaaccgaaaagcgtggtgttggtgcgatcattgtccctaaggaaaagccattggaggctatatgtcgacctcgaccctcaatggaggatttcctcgctaaaatttag

SEQ ID NO: 32 - DNA, 1476 bp, synthetic construct
  source 1..1476: mol_type = other DNA
atggagatactctacacttacctgtcgatattcgcaacattcttcttcctctacaagattatccaaagcctcaagccaaacaacaagaagcacccgccgagccctccgtcataccctatcctcgggcaccttcacctccttaagccgcctttccaccgtaccctgcaaagcctcgcacaacggtacggccccatattctcccttaagctcgggctgcaacgcgcactggtcgtaagctccgcctgggccgctgaggagtgcttctcgcagaacgatgtcgtgttcgctaaccggccgaaattcatagtggggcaacatcttggctacaaccacagcattcttatctggtcaccctacggagattactggcgtaacttgaggagggtcactactattaccatgctttcgatgaggcggatcaacgaggctggtccgactcgaaaagtcgagatccggaatatgattatggatttgcttgagtctagtaacttcggctccggcacgcagaaggtgaacatgaacgaagtattgtctaaactagcgcgcaacttcgtcatgagaatcgtgaacggcaagagttgggagaaaatgattatcaagccaccggaaaaccttatgaacatctgtgactttctcccgttcctgagatgggtggacttcaaaggaattgagaaggatctcaaggagaaacaagtcgagcgggatgagttcctccaatctctcgtggacgagattcgagagagccgtaagaagggcgaggatcttgggatgaacaccctgatcgagcagctcctggacttgcaacaagccgagcccgaccagtattctgatgagacgatcaagggcatcatactcgtgatgctcctggcgggctctgagaccacagcgcggaccctagagtgggccttgagcaatcttatcaaccaccctgagattctcgtgaaagccagaactgaaatcgaccttcatgttgggaaggaacggctggttgacgatagtgacctcccaaaacttacctacacaaggtgcatcgtttatgagacgctgcgactcttccccgccgcgccacttctggtcccgcacttctcctcgcaagactgcacgattggcggctaccacgtcccaaagggaaccatgctcttcgtgaatgcttgggccattcaccgtgatccgaccttatgggatgagcctactgtgtttaagccagagcgcttcgagaaggagaaggagggcttcaagttcatgccgttcgggattgggaggcgttcgtgcccaggcaacaacgtggcgcttcgcaatgtgtccttaaccctagcgacccttattcaatgcttcgactgggaggccgcgcagagtgggagcattgacttgactgagaaacggggagccggagctatcatcgtccctaaggagaagccactagaggcgatctgtcgcccacgtccttcaatggaggacttccttgctaagatatag

SEQ ID NO: 33 - DNA, 1470 bp, synthetic construct
  source 1..1470: mol_type = other DNA
atggaaatcttattcacctttctctcgatattcgcaacattcttcttcctgtacaagatcattcaaagtctcaagacaaacaacaagaagcacccaccatcacctccctcatatcccctgctggggcacctccacctcctcaagcctccctttcaccggactcttcaatccctggcccagcgctacgggccgatcttcacactaaagttgggactacaacgagcagttgtggtgtcctccgcctgggccgctgaggaatgcttcggccagaatgatgtggtctttgcgaatcggccgaagttcattatcggacagcatctgggatacaaccactcaatactcatctggtcgccgtatggggattactggcggaacttgaggcgcgtgaccacaattaccatgctttccatgaggaggataaacgaggctgggcccactcggaagttggagatccggaacatgattatgaacctcttggagagcgggagtggcacgcagaaggtcaatatgaacgaggtgctatccaaactcgcaaggaacttcgtgatgcgaattgtcaatggaaagtcatgggagaaaatgattatcaagcctcccgagaacttgatgacgatatgcgactttctcccgttcctaagatgggttgacttcaagggtatcgagaaggacatgaaggagaagcaagtagaacgggacgagttcctccaatcactcgtggacgagtttcgtgagtctaagaagaagggcgaggatctcggcatcaacaacacactgatcgaacaattgcttgacttgcaacaagccgagcctgaccaatactctgacgagaccatcaagggcattatcttagtgatgctgttggctggctctgagaccaccgcgaggactctcgaatgggctctgtcgaaccttatcaatcacccggagatccttgtgaaagcccgaacggagatcgacctgcatgtggggaaggagcgcctggtggacgactctgacctcccgaagctgacctacactcgttgcatcgtgtacgagacattacgcctgtttcctgccgctccgctgttagtacctcacttctctagtcaagactgtaccatcggaggctatcacgtccctaaggggactatgctattcgtaaacgcatgggccatccaccgcgacccgactctctgggacgagccgactgtcttcaagcccgagcggttcgagaaggagaaggaaggcttcaaattcatgccgtttggcatcggaaggcggtcctgccctggaaacaacgtggctctacgaaacgtcagtcttaccctcgctaccctgattcaatgtttcgactgggaggcggcggagagcggtagcatcgaccttaccgaaaagcgtggagtcggggccatcatcgtgccaaaggagaagccgcttgaggcgatctgccgtccccgtccgtctatggaggacttcctggcgaagatatga

SEQ ID NO: 34 - DNA, 1470 bp, synthetic construct
  source 1..1470: mol_type = other DNA
atggaaattctgttcacctttctctcgatattcgccacattcttcttcttgtacaaaatcatccaatcgcttaagaccaacaacaagaagcaccctccgagcccgccctcctacccgctcctgggccacttacacctcctcaagccgcccttccaccgcaccctgcaatccctcgcccagcgctacggcccgatcttcaccttgaagctgggccttcaacgcgccgtggtggtgtcatccgcctgggctgcggaggagtgcttcggccagaacgacgtggtgttcgccaaccgcccgaagttcatcatcggacagcacctgggatacaaccacagcatactcatctggagcccatacggggattactggcggaacttgcggcgcgtgaccacgataacgatgctcagtatgagacggattaacgaagcggggcccacccggaaactggagattcggaacatgattatgaacctgctggagagcggctccggcacccagaaggtcaacatgaacgaagtcctgtcgaagctggcgcgcaacttcgtgatgcggatcgtgaatggcaagtcatgggagaaaatgattatcaagccgccggagaacctgatgacgatctgcgactttctccccttcctgcgctgggtggacttcaaggggatcgagaaggatatgaaggagaagcaagtcgagagagacgagttcctgcaaagcctagtagatgagttccgagagtctaagaagaagggcgaggatctcggtattaacaacaccctgattgagcagctcctggatctccaacaagctgagccggatcaatactccgacgagaccatcaagggcatcatactcgtcatgctgctggcggggtcggagaccacggcccgaactctcgaatgggcgttgtccaaccttatcaaccaccccgagatactcgtaaaggcccgtactgagatcgacctccacgtcggcaaggagcgcttggtcgatgactccgacctgcccaagttgacttacacccgctgcatcgtttacgaaacgctccgcttattcccagcggccccacttctggtcccgcacttctcatcccaagactgcactatcggcggctaccacgtcccgaaaggcactatgctcttcgtgaatgcgtgggccatccaccgtgacccgacgctctgggacgaacctactgtcttcaagccagagcgattcgagaaggagaaggagggcttcaaattcatgcccttcgggatcggacgtcgctcgtgccccggcaacaatgtcgccctccgcaatgtgtcgctcacgctggccactctgatccaatgcttcgattgggaggccgccgagtccggcagcatcgacctgaccgagaaacgcggcgtcggggccataattgtgccaaaggagaagccattggaagcgatctgccgccctcggccctcaatggaggacttcctggccaaaatctga

SEQ ID NO: 35 - DNA, 1479 bp, synthetic construct
  source 1..1479: mol_type = other DNA
atggaaatactgttcacctttctctctatcttcgccacattcttcttcttgtacaaaatcatccactcccttaagacaaacaacaagaagcaccctccatctccgccgtccccacccagttacccgctcctcggccaccttcatctacttaagcctcccttccaccggacgttgcaatctctggcgcaaagatatgggccgatcttcactctaaagctcggtttacaacgcgcagtcgtagtgagttccgcttgggcggcggaggaatgctttgggcaaaatgatgtggtgttcgctaaccggcctaagttcatcattggacagcacttgggctacaaccacagcattctaatctggtccccgtatggggattactggcgtaacctccggcgcgtaaccaccataacgatgctctcaatgagacgcattaacgaggctggaccgacgcggaaacttgagatccgcaacatgattcgtaacctgctggagtctgggtccgggacgcagaaggtcaacatgaacgaggttctaagtaaacttgcgcggaacttcgtgatgagaatagtgaacggaaagtcgtgggagaaaatgattatcaagcctccggagaatctgatgacaatctgtgacttcctcccattcttgcgatgggtggacttcaaaggaattgagaaggacatgaaggagaagcaagtggaaagggatgagttcctgcaaagtctcgtagatgagttccgggaaagcaagaagaagggcgaagacctggggatgaacaacacccttatcgagcagcttctcgacctccaacaagctgagcctgaccagtattccgacgagactatcaagggtatcatcctagtaatgctcctggcgggatctgagacaactgccagaacgctggagtgggccctttcaaaccttattaaccacccagagatcctcgcaaaggccaggactgaaattgacttgcacgtggggaaggagcgcttggtcgatgacagcgacttgccgaagttgacatacactcgctgcatcgtttacgagacgctccgtctgttccccgcagcccctttgcttgtaccccacttctcctcccaagactgcaccatcggcggataccacgttccaaagggcacaatgctcttcgtgaacgcctgggcgatccaccgtgaccctaccctgtgggacgagcccacgctattcaagcccgagcgattcgagaaggagaaggaagggttcaagttcatgccgttcgggatcggaagacgatcttgccctggaaacaacgtcgctcttcgcaacgtctcccttacgctcgcaaccctaatccaatgcttcgattgggaggccgcagagtcgggtagcatcgacctaacggagaagcggggcgtcggggcgataatcgtgccgaaggagaaacctctggaggctatatgccgtccaagaccttctatggaggacttcctggccaaaatctga

SEQ ID NO: 36 - DNA, 1479 bp, synthetic construct
  source 1..1479: mol_type = other DNA
atggaaatactcttcacctttctctctatcttcgccacgttcttcttcttgtacaaaatcatacactccctcaagacaaacaacaagaagcacccaccctccccgccatccccaccgtcctatcccctgctggggcacctccacctactcaagcctccattccaccggacccttcaatccctggcccagcggtacggcccgatcttcaccctaaagctgggcctgcaaagagccgtcgtggtgtctagtgcctgggccgccgaggagtgcttcggccagaacgatgtcgtcttcgcaaaccgacccaagttcatcatcggccagcacctcggctacaaccacagcatcctcatctggagcccgtatggcgactattggcggaacctacggagggtcactaccatcacgatgctatctatgcgccgcatcaacgaagccggacccacgcgaaaactggagatccgtaacatgatccgcaacctgctggagagcggttcgggaacccagaaggtgaacatgaacgaagtcctatccaagctggcgcgcaacttcgtaatgcggatagtgaacggcaagagctgggagaaaatgattatcaagccaccggagaacctcatgacaatttgcgacttcctcccattcttgcgttgggtcgatttcaagggaatcgagaaggacatgaaggagaagcaagtggagagagacgagttcctgcaaagcctcgttgacgagtttcgggagtccaagaagaagggcgaggacttggggatgaacaacacactgattgagcagttgctggacctccaacaagcagagccggaccagtattctgacgagacgattaaggggatcatcctcgtcatgctgttggcgggcagtgagacgacggcgagaaccctcgaatgggccctgtccaaccttatcaaccaccccgagatcctcgcaaaggcgcgcacggaaattgatctgcacgtgggcaaggagcgcctggtggacgactccgacctcccgaagctgacctatacccgctgcatcgtttacgagactttgaggctcttccctgccgccccgttgctggtcccacacttctcaagccaagactgcaccatcggcggctaccacgtccccaaaggaactatgctgttcgtaaatgcgtgggctatacaccgagacccgaccctctgggacgaacccactctgttcaagcccgagcgcttcgagaaggagaaggagggcttcaagttcatgccgtttggcatcggtcggaggagctgtcccggaaacaacgtggcacttcgcaatgtcagcctcacgctggcgacgctcatccaatgcttcgactgggaggccgccgagtcggggtcgattgacctgaccgaaaagcggggcgtaggggccatcatcgtcccgaaggagaagccccttgaagcgatatgccgaccacgcccgagcatggaggacttcctggccaaaatctga